(19) United States
(12) Patent Application Publication
(10) Pub. No.: US 2016/0008627 A1
(43) Pub. Date: Jan. 14, 2016

(54) METHODS AND DEVICES FOR VISIBLE LIGHT MODULATION OF MITOCHONDRIAL FUNCTION IN HYPOXIA AND DISEASE

(71) Applicants: Clarimedix Inc., Boulder, CO (US); The Regents of the University of Colorado, Denver, CO (US)

(72) Inventors: John Dunning, Boulder, CO (US); Robert O. Poyton, Lafayette, CO (US); Michael H. B. Stowell, Boulder, CO (US)

(21) Appl. No.: 14/339,335

(22) Filed: Jul. 23, 2014

Related U.S. Application Data
(63) Continuation of application No. 13/076,114, filed on Mar. 30, 2011, now abandoned.
Application No. 13/076,114 is a continuation-in-part of application No. PCT/US2009/059104, filed on Sep. 30, 2009.
(60) Provisional application No. 61/114,003, filed on Nov. 12, 2008.
(60) Provisional application No. 61/101,644, filed on Sep. 30, 2008.
(60) Provisional application No. 61/374,963, filed on Aug. 18, 2010.

(51) Int. Cl. A61N 5/06

(52) U.S. Cl. CPC A61N 5/0618; A61N 2005/0626; A61N 2005/0645; A61N 2005/0653; A61N 2005/0662; A61N 2005/0667; A61N 2005/0647

(57) ABSTRACT

The present invention provides methods of using electromagnetic radiation in the visible portion of the spectrum to modulate mitochondrial function in the treatment of various conditions, including Alzheimer's disease, other demential, hypoxia and diabetic peripheral neuropathy, and sensory disorders of the extremities.

### CROSS-REFERENCE(S) TO RELATED APPLICATION(S)

This application is a continuation-in-part of International Patent Application No. PCT/US2009/059104, accorded an international filing date of Sep. 30, 2009, which claims the benefit under 35 U.S.C. §119(e) of U.S. Provisional Patent Application No. 61/101,644 filed Sep. 30, 2008, and U.S. Provisional Patent Application No. 61/114,003 filed Nov. 12, 2008. This application claims the benefit under 35 U.S.C. §119(e) of U.S. Provisional Patent Application No. 61/374,963 filed Aug. 18, 2010, where this International Patent Application and these (three) provisional applications are incorporated herein by reference in their entireties.

### STATEMENT OF GOVERNMENT INTEREST

This invention was made with government support under Grant No. GM30228 awarded by the National Institutes of Health. The government has certain rights in this invention.

### BACKGROUND OF THE INVENTION

Photobiomodulation, using light emitting diode (LEDs) arrays or low energy lasers, has been reported to have a variety of therapeutic benefits (Conlan et al. 1996; Sommer et al. 2001; Whelan et al. 2001; Yu et al. 1997; Delellis et al. 2005; Powell et al. 2004; Harkless et al. 2006; and Powell et al. 2006). This non-invasive therapy has been used to accelerate wound healing, improve recovery rates from ischemia, slow degeneration of injured optic nerves, and improve sensitivity and reduce pain in various types of peripheral neuropathies including those associated with diabetes.

Diabetes is a common metabolic disorder that is rapidly becoming an epidemic worldwide (Lowell and Schulman, 2005). In the United States, Type II diabetes is the leading cause of blindness. Diabetic peripheral neuropathies are some of the most common long-term complications of diabetes (Pop-Busui et al. 2006). They are a major cause of pain associated with diabetes and often result in lower extremity amputations. Although studies have reported that many patients with diabetic peripheral neuropathies are responsive to near infrared radiation (NIR) therapy (Delellis et al. 2004; Powell et al. 2004; Harkless et al. 2006; Powell et al. 2006), the therapeutic mode of action of photobiomodulation in treating these neuropathies is not yet clear.

NIR is effective in these therapies. Light in the NIR has significant advantages over visible or ultraviolet light because it penetrates tissues more deeply than visible light and at the same time lacks the carcinogenic and mutagenic properties of ultraviolet light (Whelan et al. 2001, 2002). The cellular and molecular mechanisms that underlie the therapeutic benefits of NIR are still poorly understood. Several studies have indicated that the most effective wavelengths for therapeutic photobiomodulation are somewhere between 600 and 830 nm (Karu, 1999; Karu et al. 2005) and that mitochondrial Cco is the primary photoreceptor for photobiomodulation (Eells et al. 2004; Karu 1999; Karu et al. 2005; Wong-Riley et al. 2005).

Until recently, mitochondrial cytochrome c oxidase was thought to have only one enzymatic activity; the reduction of oxygen to water. This reaction occurs under normoxic conditions and involves the addition of 4 electrons and 4 protons to diatomic oxygen. During this process oxygen is reduced by a series of one electron transfers. The first electron added to oxygen produce superoxide (02), the second electron produces peroxide (H202), the third electron added produces the hydroxyl ion (OH′), and the fourth electron produces water. Superoxide, hydrogen peroxide, and the hydroxyl ion are incompletely reduced forms oxygen and are referred to collectively as reactive oxygen species (ROS). ROS are normally sequestered at the binuclear reaction center within the holocytochrome c oxidase molecule and are not released. However, under some pathological conditions (Poyton, 1999) they are released and can either act destructively (to induce oxidative stress, a condition that lies at the heart of many diseases as well as aging), or constructively (in intracellular signaling pathways (Poyton and McEwen, 1996)). Because light can affect the oxidation state of cytochrome c oxidase (Winterrle and Einarsdottir, 2006, Tachtsidis et al. 2007), it can also alter the conformation of the binuclear reaction center and cause the release of reactive oxygen species.

It is now clear that the mitochondrial respiratory chain and mitochondrial cytochrome c oxidase can have profound effects on cell growth, aging, and the induction of a large number of nuclear genes when cells experience low oxygen levels (Poyton and McEwen, 1996; Castello et al. 2006; Ryan and Hoogenraad, 2007). These effects are brought about by signaling pathways between the mitochondrion and nucleus. Although these pathways are still incompletely understood, there is now compelling evidence that superoxide (0₂) nitric oxide (NO), and peroxynitrite (ONOO⁻) (formed by the reaction of NO with 0₂) are involved. The peroxynitrite generated from NO and superoxide is capable of affecting protein tyrosine nitration, which, in turn, may alter specific proteins involved in mitochondrial-nuclear signaling pathways.

In order to better understand and treat disease by photobiomodulation, it is important to identify important quantifiable biomarkers that are affected by the disease and subsequently altered by light therapy. This invention provides for these and other needs by disclosing such predictive biomarkers but also in using them to determine the wavelengths of radiation most suitable for phototherapy.

### BRIEF SUMMARY OF THE INVENTION

In various aspects, the invention relates to the use of visible electromagnetic radiation to modulate NO production and to reduce the level or production of reactive oxygen species in hypoxia. In other aspects, the invention relates to the absorption of visible light by cytochrome c mediating the effect of electromagnetic radiation on mitochondria and that the wavelength(s) of electromagnetic radiation to use in modulating mitochondrial function are those wavelengths preferentially absorbed by cytochrome c oxidase. In preferred embodiments, accordingly, the effects of the radiation are mediated by the absorption of the visible light by cytochrome c oxidase. In other embodiments, the effects of the electromagnetic radiation (e.g., visible and near infrared radiation) are mediated by the ability of the radiation to promote the phosphorylation or conversion of cytochrome c oxidase into a form which more readily generates NO.

Accordingly, in a first aspect, the invention provides a method of treating hypoxia in a tissue of a mammalian subject by diagnosing the hypoxia or a condition associated with hypoxia and exposing the hypoxic tissue of the mammal to electromagnetic radiation. Exposure to the radiation improves tissue blood flow in the hypoxic state by increasing the production of NO thereby reducing vascular resistance in the tissue. Accordingly, in one embodiment, the invention provides a method of preventing or repairing tissue damage in a hypoxic tissue by exposing the tissue to electromagnetic radiation. In related embodiments, the invention provides methods of increasing mitochondrial nitrite reductase activity or NO production in the exposed tissue by exposing the tissue to electromagnetic radiation. In some embodiments, the invention provides an in vivo or in vitro method of modulating NO production by neurons or endothelial cells in a mammalian tissue capable of producing NO under hypoxic conditions and/or high concentrations of glucose by cyctochrome c oxidase nitrite reductase activity by exposing the neurons or endothelial cells to the radiation. In another embodiment, the invention relates to combination therapy of electromagnetic radiation with a second agent (e.g., nitrite, NO donors, nitroglycerin, organic nitrites, arginine) which promotes NO activity in reducing vascular resistance. In preferred embodiments, of any of the above, the radiation is in the visible portion of the electromagnetic radiation spectrum. In some embodiments, the hypoxia is as low as 1-2% to 80%, 2 to 10%, about 10% to 80% or 10% to 50% normoxia for a given tissue; in other embodiments, it is from 10% to 30% normoxia for a given tissue, or 10-15% normoxia for a given tissue. In other embodiments, the hypoxia corresponds to 20 to 100 micromolar oxygen, 20 to 80 micromolar oxygen, 20 to 50 micromolar oxygen, or 22 to 35 micromolar oxygen (e.g., 30 to 50 torr) in a tissue (e.g., blood).

In a second aspect, the invention provides a method of improving energy metabolism in a hypoxic tissue by exposing the tissue to electromagnetic radiation. The exposure to electromagnetic radiation alters cytochrome c oxidase or the phosphorylation of cytochrome c oxidase in such a way as to modulate its nitrite reductase activity. Additionally, the electromagnetic radiation exposure leads to the increased expression of mitochondrial proteins, leading to an increase in mitochondrial biogenesis in the tissue. In some related embodiments, the invention provides a method of modulating respiration mediated by cytochrome c oxidase in a cell of a tissue or of modulating the phosphorylation of cytochrome c oxidase in a cell of a tissue by exposing the tissue to electromagnetic radiation. In some embodiments, the amount or expression of one or more subunits selected from the group of subunits of cytochrome c oxidase, cytochrome c, cytochrome c reductase or ATP synthetase in the tissue is increased.

In a third aspect, the invention provides a method of reducing oxidative stress or toxic stress in a tissue of a mammal by exposing the tissue to electromagnetic radiation. In some embodiments, there is a reduction in any one or more of induced oxidative stress genes, levels of lipid peroxides, oxidized nucleosides and oxidized amino acids or polypeptides in the tissue. In some embodiments, the toxic stress is caused by exposure to a chemical which is metabolized to a reactive species or to generate an oxygen radical.

In a fourth aspect, the invention provides a method of monitoring the effect of treatment with electromagnetic radiation on a mammalian subject, said method comprising exposing a tissue of the subject to electromagnetic radiation and measuring the effect of the radiation on the production of NO on NO-induced vasodilators by the tissue.

In a fifth aspect, the invention provides a method of prognosis and/or diagnosis for poor blood circulation or diabetic peripheral neuropathy (DPN) in a tissue or organ, said method comprising measuring the tissue or blood NO, VEGF, or protein carbonylation levels. In some embodiments, the NO and VEGF levels indicate early stage DPN prior to loss of sensation and pain.

In a sixth aspect, the invention provides a method of treating a mammalian subject for diabetic peripheral neuropathy said method comprising exposing an affected tissue to electromagnetic radiation.

In a seventh aspect, the invention provides a method of monitoring the response to exposure of a tissue to electromagnetic radiation by measuring blood flow in the tissue, or measuring the tissue or blood NO, VEGF, or protein carbonylation levels. In some embodiments, the response is a response according to a method of any of aspects one through six above.

In some aspects, the invention provides methods of reducing ROS in a tissue by exposing the tissue to electromagnetic radiation.

In some aspects, the invention provides methods of improved control of hyperglycemia or blood glucose levels in diabetes patients by exposing the subject o electromagnetic radiation. In some aspects, the invention provides methods of treating a neurodegenerative condition or a peripheral neuropathy by exposing the subject to electromagnetic radiation in the visible radiation range.

In some embodiments, the invention provides methods for treating diseases or conditions which may be exacerbated or caused by hypoxia or oxidative stress. Such disease or conditions include neurodegenerative diseases (such as Parkinson's disease), Huntington's disease, other neurological/degenerative disease such as Alzheimer's disease, fronto-temporal dementia, stroke, non-diabetic peripheral neuropathies and dementia; macular degeneration; traumatic brain injury; ischemia/reperfusion disease; migraine; tissue injury; cardiovascular diseases including atherosclerosis and hypertension, diabetes and diabetic complications of the eye (e.g., macular degeneration), kidney, and nerves (e.g., diabetic peripheral neuropathy); inflammation, arthritis, radiation injury, aging, burns/wound healing; spine/back disease such as herniated discs; peripheral vascular disease, and vasospasm. In some embodiments, the invention also provides methods for treating obesity.

In some embodiments of each of the above aspects and embodiments, the wavelength of electromagnetic radiation or light to be used is visible radiation. Accordingly, in such embodiment, the wavelength of electromagnetic radiation light to be used comprises wavelengths from about 400 to 625 nm, 500 to 650 nm, from 550 to 625 nm, from 575 nm to about 625 nm in wavelength, or from 500 to 600 nm, 550 to 600 nm, from 575 to 600 nm. In some further embodiments of the above, the wavelength of electromagnetic radiation to be used is substantially free of light having a wavelength greater than 595 nm, 600 nm, 610 nm, 615 nm, 625 nm, 630 nm, 650 nm, or 675 nm. In yet other embodiments, the applied electromagnetic radiation is substantially free of radiation in the 615 to 750 nm range, the 620 to 700 nm range, 630 to 700 nm range, 630 to 750 nm range, 630 to 675 nm range, the 650 and 700 nm range, or 625 to 800 nm range.

In some embodiments, the wavelengths of light used falls within or are principally comprised of wavelengths falling within the primary band of mitochondrial cytochrome c oxidase. In some embodiments, the wavelengths of light used fall within the band of such wavelengths stimulating production of NO by cytochrome c oxide. In some embodiments, the light or radiation specifically targets the haem absorption bands of cyctochrome c oxidase. In further embodiments of such, the wavelengths of light are free or substantially free of wavelengths which inhibit the product of NO by cytochrome c oxidase. The period and/or intensity and/or intensity of this light can be adjusted to fit the individual subject or therapeutic objective as described further herein.

In some embodiments of any of the above, there is a proviso that the mammalian subject does not have diabetes. In some embodiments of any of the above, there is a proviso that the tissue is not diabetic or is not affected by DPN.

The above methods can stimulate NO production in treated tissue. Accordingly, in a further aspect of any of the above, the invention further provides for a combination therapy comprising use of any one of the above methods in combination with therapy to modulate NO activity in the subject. This therapy may include administration of NO donors and other compounds (substrates for NO synthetase, inhibitors of NO degradative pathways) which modulate NO levels in a subject.

In some embodiments, the invention provides methods of improving cognition or memory, or treating deficits in cognition or memory, or treating neurodegenerative diseases including, but not limited to, Parkinson's disease, Huntington's disease, other neurological/degenerative disease such as Alzheimer's disease, fronto-temporal dementia, stroke, non-diabetic peripheral neuropathies and dementias; senile or aged dementia, macular degeneration; traumatic brain injury; ischemia/reperfusion disease by exposing the subject to electromagnetic radiation in the visible portion of the spectrum. In some embodiments, the radiation is substantially in the range from 500 to 600 nm, and more preferably from 550 to 600 or, still more preferably, from 570 to 590 nm. In further embodiments, the radiation is provided by targeting the electromagnetic radiation to the carotid or other cerebral arteries. The radiation can be principally composed of monochromatic or polychromatic light. The light source can be preferably an LED or, more preferably, an organic LED. The dosage regimen (e.g., site, surface area, period and/or intensity/power and/or duration of this radiation exposure) can be adjusted to fit the individual subject or therapeutic objective as described further herein.

In a further related embodiment, the present invention provides a medical treatment device, comprising: a patch wearable by a mammal, the patch having a tissue facing surface and including a light source having a plurality of organic light emitting diodes, the light source operable to emit outwardly from the tissue facing surface electromagnetic radiation in a visible portion of the electromagnetic spectrum from about 375 nm to about 650 nm or from about 375 nm to about 625 nm, which is substantially free of at least a portion of electromagnetic radiation in at least the near infrared portion of the electromagnetic spectrum above about 650 nm. In certain embodiment, the light source is operable to emit the electromagnetic radiation in the visible portion of the electromagnetic spectrum with at least one of a surface power density of about 10 mW/cm² to about 10 W/cm² or a total power output of about 25 mW to about 100 W measured adjacent to the tissue facing surface. In certain embodiments, the tissue facing surface of the patch has a surface area of from about one square inch to about ten square inches. In various embodiments, the patch is conformable to a neck of a human In certain embodiments, the tissue facing surface of the patch has a diameter of about 0.5 to about 4.0 inches. In particular embodiments, the patch further comprises a biocompatible adhesive carried by the patch to selectively removably attach the patch to the mammal or a bodily tissue of the mammal. In certain embodiments, the device further comprises a battery integral to the patch and electrically coupled to supply electrical power to the plurality of organic light emitting diodes. In certain embodiments, the battery is sized to provide only a single-use treatment. In som embodiments, the device further comprises a controller coupled to selectively control the plurality of organic light emitting diodes. In particular embodiments, the controller is configured to pulsate the organic light emitting diodes. In certain embodiments, the device further comprises at least one sensor positioned to sense at least one parameter of a treatment and communicatively coupled to the controller to provide signals thereto, wherein the controller is configured to adjust at least one operational parameter based on the signals from the at least one sensor. In particular embodiments, the treatment parameter that the at least one sensor senses includes at least one of a patient characteristic, a selected applied power density, a target time interval, a power density/timing profile, or a temperature. In certain embodiments, the device further comprises one or more optical filters that remove at least a portion of the electromagnetic radiation having wavelengths greater than about 650 nm. In various embodiments, the light source is operable to emit outwardly from the tissue facing surface the electromagnetic radiation in the visible portion of the electromagnetic spectrum from about 375 nm to about 650 nm, or from about 375 nm to about 650 nm, and which is substantially free of wavelengths greater than about 675 nm, and which has a peak energy transmission at or within 10 nm of a wavelength of about 400 nm, 550 nm, about 560 nm, about 570 nm, about 580 nm, about 590 nm, about 600 nm, or about 610 nm; or which has an energy distribution for which 80% or 90% if the energy is found within the wavelengths of 500 nm to 625 nm.

In a further related embodiment, the present invention provides a method comprising supplying a device comprising a light source, wherein the light source comprises a plurality of organic light emitting diodes, and wherein the light source is in the form of a multi- or single-use patch; and operating the device to emit electromagnetic radiation in a visible portion of the electromagnetic spectrum from about 375 nm to about 625 nm substantially free of at least a portion of electromagnetic radiation in at least the near infrared portion of the electromagnetic spectrum above about 650 nm. In certain embodiments, operating the device includes: operating the device to emit the electromagnetic radiation in the visible portion of the electromagnetic spectrum with at least one of a surface power density of about 10 mW/cm² to about 10 W/cm² or a total power output of about 25 mW to about 100 W measured adjacent to a surface of the device; causing a controller to selectively control operation of the plurality of organic light emitting diodes; operating the device to emit the electromagnetic radiation for a period of time from about 10 seconds to about two hours or more; and/or operating the device to emit the electromagnetic radiation continuously or at a pulse frequency of about 4 to about 10,000 Hz. In particular embodiments, supplying the device includes: supplying the device comprising the light source in the form of a multi- or single-use patch having at least one of a surface area of from about one square inch to about ten square inches or a diameter of about 0.5 inches to about 4.0 inches, and bearing an adhesive substance on an exterior surface thereof; supplying the device comprising one or more optical filters to remove a portion of electromagnetic radiation having wavelengths greater than about 650 nm; and/or supplying the device comprising: the light source comprising the plurality of organic light emitting diodes arranged in an array; and a controller coupled to selectively control the one or more organic light emitting diodes.

In another embodiment, the present invention includes a method for increasing mitochondrial nitrite reductase activity, increasing Cytochrome c oxidase activity, increasing nitric oxide production, or increasing tissue blood flow in a tissue of a mammalian subject, comprising: exposing said tissue to electromagnetic radiation in a visible portion of the electromagnetic spectrum from about 375 nm to about 650 nm, or from about 375 nm to about 625 nm, substantially free of at least a portion of electromagnetic radiation in at least the near infrared portion of the electromagnetic spectrum above about 650 nm, by externally applying the electromagnetic radiation to the mammalian subject using a medical treatment device comprising a patch wearable by the subject, the patch having a tissue facing surface and including a light source having a plurality of organic light emitting diodes, the light source operable to emit outwardly from the tissue facing surface electromagnetic radiation in a visible portion of the electromagnetic spectrum from about 375 nm to about 650 nm which is substantially free of at least a portion of the electromagnetic radiation in at least the near infrared portion of the electromagnetic spectrum above about 650 nm. In certain embodiments, the tissue is a hypoxic or ischemic tissue, a tissue affected by diabetic peripheral neuropathy, a tissue of the central nervous system, including brain tissue or spinal cord tissue, a tissue affected by hypoxia, ischemia, oxidative stress or neurodegeneration, or a tissue located some distance from the tissue affected by hypoxia, ischemia, oxidative stress or neurodegeneration. In various embodiments, the tissue is exposed to about 0.5 to about 40 joules/cm² of the electromagnetic radiation; the tissue is exposed to about 1 to about 20 joules/cm² of the electromagnetic radiation; the tissue is exposed to a power density of the electromagnetic radiation of about 0.01 mW/cm² to about 1 W/cm²; the tissue is exposed to a power density of the electromagnetic radiation of about 0.01 mW/cm² to about 100 mW/cm²; the tissue is exposed to a power density of the electromagnetic radiation of about 0.5 mW/cm² to about 8 mW/cm²; the tissue is exposed to electromagnetic radiation modulated or pulsed at a frequency of about 4 Hz to about 10,000 Hz; the tissue is exposed to the electromagnetic radiation over a treatment period of from about 10 seconds to about two hours or more in length; or the tissue is exposed to the electromagnetic radiation at a frequency of treatment of once- or twice-a-day, 1-, 2-, 3-, 4-, or 5-times a week, or once- or twice-a-month. In certain embodiments, the subject is also administered a compound that modulates nitric oxide levels in said subject.

In a further embodiment, the present invention includes a method for treating or preventing reduced blood flow, hypoxia, ischemia, oxidative stress, or neurodegeneration, or for increasing cerebral blood flow, in a mammalian subject, comprising: externally applying electromagnetic radiation in a visible portion of the electromagnetic spectrum from about 375 nm to about 650 nm substantially free of at least a portion of electromagnetic radiation in at least the near infrared portion of the electromagnetic spectrum above about 650 nm to said subject using a medical treatment device, comprising: a patch wearable by the subject, the patch having a tissue facing surface and including a light source having a plurality of organic light emitting diodes, the light source operable to emit outwardly from the tissue facing surface electromagnetic radiation in a visible portion of the electromagnetic spectrum from about 375 nm to about 650 nm, or from about 375 nm to about 650 nm, which is substantially free of at least a portion of electromagnetic radiation in at least the near infrared portion of the electromagnetic spectrum above about 650 nm. In certain embodiments, the mammalian subject has a disease or disorder selected from the group consisting of: stroke; cerebral ischemia; migraine; multiple sclerosis; amylotrophic lateral sclerosis; epilepsy; Alzheimer's disease; dementia, including Alzheimer-type dementia, cerebrovascular dementia, senile dementia, fronto-temporal dementia, and dementia resulting from AIDS; traumatic brain injury; physical trauma to the central nervous system, including traumatic brain injury, crush or compression injury to the brain, spinal cord, nerves, or retina; a neurodegenerative disease; Parkison's disease; Huntington's disease; ischemia/reperfusion disease; tissue injury; cardiovascular diseases, including atherosclerosis and hypertension; non-diabetic peripheral neuropathies; diabetes and diabetic complications of the eye (e.g., macular degeneration), kidney, and nerves, including diabetic peripheral neuropathy; non-diabetic peripheral neuropathies; inflammation; arthritis; radiation injury; aging; burns; spine/back disease, including herniated discs; peripheral vascular disease; vasospasm; a deficit in cognition or memory; and obesity. In certain embodiments, the subject has a neurodegenerative disease or disorder and the subject's brain or one or more of the subject's carotid arteries and/or vertebral arteries is exposed to the electromagnetic radiation by positioning the device on the subject's head or neck, or under the ear or behind the jaw bone of the subject. In certain embodiments, the subject has Alzheimer's disease and one or more of the subject's carotid arteries and/or vertebral arteries is exposed to the electromagnetic radiation by positioning the device on the subject's neck or under the ear or behind the jaw bone of the subject. In various embodiments of methods of the present invention, the electromagnetic radiation has a bandwidth of about 50 nm; the light source provides a unit dose of electromagentic radiation in an amount of from about 0.5 to about 40 joules/cm² per treatment; the light source provides a unit dose of electromagnetic radiation in an amount from about 5 to about 50 joules/cm²/day; the electromagnetic radiation is monochromatic light; the electromagentic radiation principally comprises wavelengths from 550 nm to 600 nm; the electromagnetic radiation principally comprises wavelengths from 575 nm to 600 nm; the subject is contacted with the electromagnetic radiation over a treatment period of from about 10 seconds to about two hours or more in length; the subject is contacted with the electromagnetic radiation from once- or twice-a-day; 1-, 2-, 3-, 4-, or 5-times a week, or once- or twice-a month; the electromagentic radiation has a peak energy emission at a wavelength of about 400 nm, 500 nm, 510 nm, 520 nm, 530 nm, 540 nm, 550 nm, 560 nm, 570 nm, 580 nm, 590 nm, 600 nm, 610 nm, or within 10 nm of any one of these values; the electromagnetic radiation has an energy distribution for which 80% of the energy is found within the wavelengths of 550 nm to 600 nm; or the electromagnetic radiation has an energy distribution for which 90% of the energy is found within with the wavelengths of 550 n to 600 nm. In particular embodiments, the subject is also administered a compound that modulates nitric oxide levels in the subject.

### BRIEF DESCRIPTION OF THE DRAWINGS

FIG. 1. Model relationships between hyperglycemia, hypoxia, vasoconstriction and photobiomodulation. Elements of this model are as follows: (1) the increased blood glucose levels in diabetes patients promotes endothelial cell aerobic fermentation reactions which promote hypoxia. (2) Under hypoxic conditions, the levels of reactive oxygen species, especially superoxide, increase. (3) This superoxide reacts with NO in the blood to produce peroxynitrite. (4) The production of peroxynitrite from blood NO effectively reduces the concentration of NO in the blood, and results in protein nitration. (5) Because NO is a vasodilator, reduction in blood NO levels results in the constriction of blood vessels (especially microvasculature).

FIG. 2. Spectral emission from a 50 watt Xenon/Halogen flood light (Feit Electric Co.).

FIG. 3. Two experimental conditions tested for nitrite-dependent nitric oxide production in yeast cells.

FIG. 4. Light stimulated nitrite-dependent production of NO.

FIG. 5. Comparison of the effects of light intensity and a respiratory chain on light-stimulated nitric oxide production in yeast cells.

FIG. 6. Power dependence of late phase light-stimulated nitric oxide production in yeast cells.

FIG. 7. Overall rates of nitric oxide production during the late phase as a function of wavelength.

FIG. 8. Model depicting intra and extra cellular actions of NO.

FIG. 9. Model depicting a suitable placement of LED device for purposes of phototherapy affecting CNS.

FIG. 10. Nitrite-dependent NO synthesis in HMVEC cells begins only when the oxygen concentration drops to less than 10 micromolar.

FIG. 11. Cytochrome c oxidase in mitochondria from HUVEC cells catalyzes nitrite-dependent NO synthesis.

FIG. 12. Nitrite-dependent NO synthesis by mouse cerebrum mitochondrial cytochrome c oxidase. Time of addition of nitrite and PTIO are indicated.

FIGS. 13A and 13B. Effects of broadband light on Cco/NO activity in HUVEC cells. FIG. 13A depicts two different experimental conditions, and FIG. 13B shows nitric oxide production under each of these conditions or without light.

FIG. 14. Effects of light intensity on cerebrum mitochondrial cytochrome c oxidase NO synthesis.

FIG. 15. Differential effects of light at different wavelengths on cerebrum mitochondrial cytochrome c oxidase NO synthesis.

FIG. 16. Cco/oxidase activity (FIG. 16A) and nitrite reductase activity (FIG. 16B) in mitochondria from 5×FAD and control mice between the ages of 6 and 8 weeks. The figure legend from top to bottom corresponds to the bars for each grouping from left to right, with the exception of no data available for wild type, 8 weeks old, in FIG. 16B.

FIG. 17. Both HNE and H202 increase APP processing to A131-42 which is reversible upon light treatment.

FIG. 18. HNE increases APP processing to A131-40 which is reversible upon light treatment.

FIG. 19. Baseline performance for each of 4 groups on the DNMP. The y-axis indicates mean daily correct response out of a maximum of 6 trials.

FIG. 20. Mean correct responses as a function of treatment day. Each score is the combined score for all four groups, and the average of all three delays. The results demonstrate that overall accuracy was greatest on the second treatment day, which followed the delivery of light therapy.

FIG. 21. Response accuracy as a function of treatment day and treatment level in the first test block. Note that all three groups receiving the light therapy responded maximally on the day the therapy was administered. The figure legend from top to bottom corresponds to the bars for each group from left to right.

FIG. 22. Mean correct out of maximum of 6 in second treatment phase. The data are plotted as a function of test day with the pretreatment day being the last test day of Block 1.

FIG. 23. Phase z response accuracy as a function of test day and treatment group. Note that all three treatment groups responded maximally on treatment day. The figure legend from top to bottom corresponds to the bars for each test day from left to right.

FIG. 24. Mean correct responses as a function of treatment day. Each score is the combined score for all four groups. The results demonstrate that overall accuracy was poorest on the second treatment day, which is the day that the testing followed the delivery of light therapy, and highest on the last treatment day.

FIG. 25. Effect of level of distractor and time post treatment on performance on the attention task. The results reflect the combined data from the same and different task. The solid line shows the data obtained one hour following treatment, and the dashed line shows the data from three hours post-treatment.

FIG. 26. Mean accuracy as a function of treatment group on the “Different” task.

FIG. 27. Mean accuracy as a function of treatment group on the “Same” task.

FIG. 28. Mean accuracy as a function of treatment group on the “Different” task.

FIG. 29. Performance of each of the groups on successive daily tests with 1 distractor (FIG. 29A), 2 distractors (FIG. 29B) and 3 distractors (FIG. 29C).

FIG. 30. Performance of each of the groups on successive daily tests with 1 distractor (FIG. 30A), 2 distractors (FIG. 30B) and 3 distractors (FIG. 30C) of the “Different” version of the attention task.

FIG. 31. Performance as a function of baseline, delay and treatment group on DNMP task. The figure legend from top to bottom corresponds to the bars for each delay group from left to right.

FIG. 32. Depictions of an exemplary OLED device stack, an exemplary OLED energy diagram, an exemplary generic fabrication structure of OLEDS, and exemplary materials used in OLEDs.

FIG. 33. Chemical structure of GT3-105, Nile Red, PVK, and PEDOT:PSS, with the electroluminescence spectra of the FT3-105 (solid) and Nile Red/PVK (dashed) devices under forward bias conditions shown.

FIG. 34. Study design (FIG. 34A) and addendum study design (FIG. 34B).

### DETAILED DESCRIPTION OF THE INVENTION

In certain embodiments, the present invention relates to the use of electromagnetic radiation in the visible portion of the spectrum to modulate cytochrome c oxidase (Cco), cytochrome c oxidase phosphorylation and, also, particularly, to modulate the ability of mitochondria to make NO, and additionally, the ability of this NO to modulate circulation in a tissue exposed to the electromagnetic radiation. The mitochondrion, and more particularly, cytochrome c oxidas, is a major control point for cell energy production (Poyton, 1988). Accordingly, TER modulation of cytochrome c oxidase and mitochondrial function can also produce signal molecules that provide immediate benefits to cell and tissue function in hypoxic tissue. Additionally, electromagnetic radiation in the visible portion of the spectrum is useful in modulating cell viability or reproduction in hypoxic tissue and protecting cells and tissues from hypoxia. Whereas the former effects should have immediate short-term effects on cell and tissue physiology, the latter effects would be expected to have more long-term effects.

Certain embodiments of the invention relate to the Applicants' findings that:
Light has a specific effect on gene expression in hypoxic yeast cells in the presence of nitrite. This increase correlates with an increase in respiration, suggesting that light stimulates the ability of cells to make the energy that can fuel cell growth and division.
Mitochondrial cytochrome c oxidase in human endothelial and mouse neuronal cells catalyzes nitrite-dependent NO synthesis under those hypoxic conditions that accompany some pathophysiological states.
Broadband light has a marginal effect on cytochrome c oxidase nitrite-dependent NO synthesis in endothelial cells but a significant effect on this reaction in mitochondria from the cerebrum of mouse cells. Light acts in a dose-dependent fashion light on these mitochondria. Those wavelengths that are most effective in stimulating this reaction are: 400±25 nm, 500±25 nm, 550±25 nm, and 600±25.
The level of mitochondrial cytochrome c oxidase (Cco) activity in mouse cerebrum declines with age both in wild type mice and in a 5×FAD mouse model for Alzheimer's disease. However, the nitrite reductase activity of Cco doesn't decline with age either in the wild type mouse of the 5×FAD mouse. Light stimulates this activity in both wild type and 5×FAD mice but its ability to do so declines slightly with age, between 6 and 8 weeks, in the 5×FAD mouse.

These results provide support for the use of light to modulate mitochondrial NO synthesis and in the therapeutic photobiomodulation of mitochondrial function as it relates to disease in general, including, but not limited to, metabolic disorders mediated by, or characterized by, impaired mitochondrial function, aging, neurogenerative diseases associated with aging, (e.g., Parkinsons' disease and Alzhemier's disease). In some embodiments, the methods provide means for improving blood flow in hypoxic tissues, and reducing blood pressure.

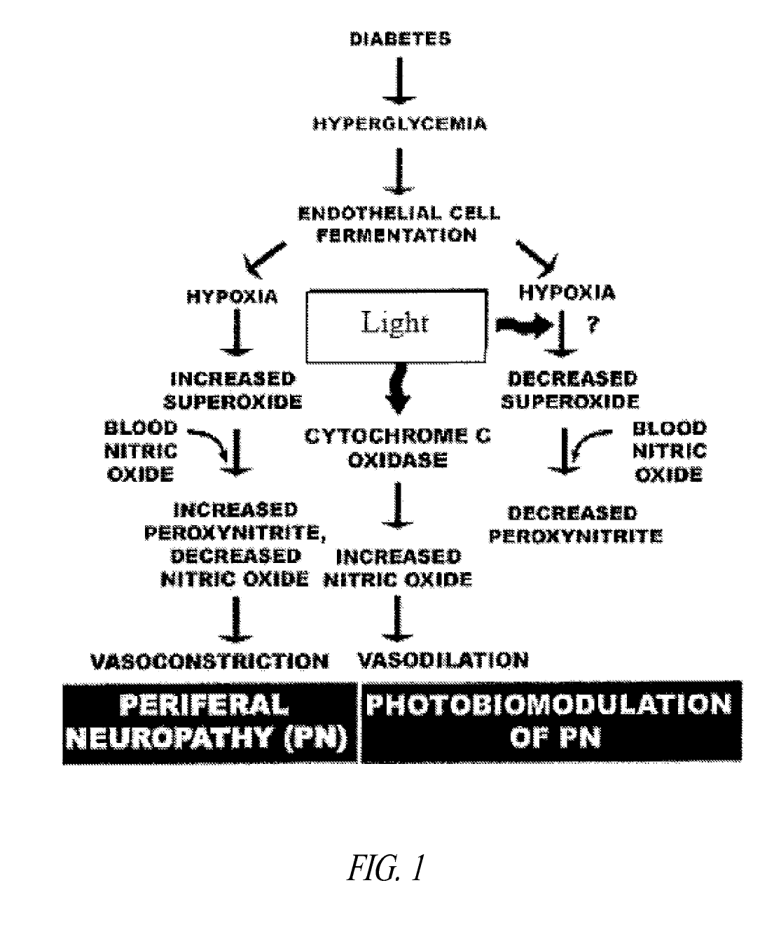

The term “modulate” means to decrease or increase. The modulatory stimulus may be dynamic (varying over time) during application or constant. The visible light modulation of mitochondrial function is illustrated in FIG. 1. The modulation can be therapeutic in nature and result in the treatment (e.g., amelioration, reduction (as to either frequency or severity) or prevention (e.g., delay in on-set or failure to develop) of the recited adverse condition or the signs, symptoms, or adverse sequelae of the recited adverse condition. Modulation can also promote the health of a tissue or subject with respect to a particular condition.

Much previous research has focused on the effect of light on mitochondria under conditions of normal oxygen tension. The results of these studies has indicated that Near Infrared Radiation (NIR) was particularly suitable for protecting mitochondrial function. The present invention relates to the surprising finding that 1) anoxic mitochondrion also produce ATP with nitrite as an electron acceptor; 2) light in the visible portion of the spectrum promotes the production of NO by mitochondria under these conditions; and 3) light in the NIR which promotes ATP function in mitochondria under normal oxygen tension actually inhibits the ability of mitochondria to produce NO. As NO is a potent vasodilator, the switch to NO production is beneficial in helping restore blood flow and normal oxygen tension to hypoxic or anoxic tissue. Accordingly, the Applicants' discoveries provide new methods for treating a number of conditions where increased NO production or enhanced bloodflow would be beneficial.

Particular embodiments of the invention relate to the Applicants' discovery that visible light falling within the wavelength range of 400 to 625 nm (e.g., 400±25 nm, 500±25 nm, 550±25 nm, and 600±25), including 550 to 625 nm, benefits mitochondrial function under anoxic conditions and that light within a wavelength range of about 625 nm to 750 nm inhibits this therapeutic effect. Accordingly, in some embodiments, the invention provides for improved methods of promoting mitochondrial function under conditions of reduced oxygen by applying to a target tissue monochromatic or polychromatic light of a wavelength from about 400 to 625 or 550 nm to 625 nm. In some further embodiments, this light is substantially free of electromagnetic radiation having longer wavelengths or free of radiation having a wavelength from about 630 nm to 700 nm in wavelength.

Accordingly, in one aspect, the invention provides methods of treating hypoxia in a tissue of a mammalian subject, said method comprising exposing the hypoxic tissue of the mammal to electromagnetic radiation in the form of visible light. In some embodiments, the response to the treatment is assessed by measuring the blood flow of the affected tissue. In others, blood or tissue levels of NO or a NO-induced vasodilator or VEGF is monitored to assess the response to the treatment. In preferred embodiments, the radiation increases NO production by mitochondria of the exposed tissue and blood flow in the exposed tissue increases. In other embodiments, mitochondrial oxygen efficiency in the exposed tissue is increased by the exposure. In some embodiments, the hypoxia is due to poor circulation of the extremities. In exemplary embodiments, tissue is that of a subject with diabetes. In other embodiments, the treatment alleviates a sign or symptom of peripheral neuropathy in diabetic or non-diabetic patients on in patients with normal glucose control. In some embodiments of such, the treatment alleviates sensory disturbances (e.g., pin and needle sensation, numbness, burning, or other unpleasant sensations) in the extremities (e.g., feet or hands).

In another aspect, the invention provides a method of treating a mammalian subject for diabetic peripheral neuropathy by exposing an affected tissue of the subject to electromagnetic radiation in the visible portion of the spectrum. In yet another aspect, the invention provides a method of improving energy metabolism in a hypoxic tissue by exposing the tissue to this radiation. In still another aspect, the invention provides a method of reducing oxidative stress in a tissue of a mammal by exposing the tissue to electromagnetic radiation in the visible portion of the spectrum. In some embodiments of any of the above, there is a reduction in any one or more of induced oxidative stress genes, levels of lipid peroxides, oxidized nucleosides and oxidized amino acids or polypeptides in the tissue.

In a further aspect, the invention provides a method of modulating respiration mediated by cytochrome c oxidase in a cell of a tissue or of modulating the phosphorylation of cytochrome c oxidase in a cell of a tissue by exposing the tissue to electromagnetic radiation in the visible portion of the spectrum. In another aspect, the invention provides a method of modulating mitochondrial function in a tissue, said method comprising exposing the tissue to electromagnetic radiation in the visible portion of the spectrum.

In some embodiments of any of the above aspects, there are further embodiments in which the modulation increases mitochondrial nitrite reductase activity, NO production in the exposed tissue or mitochondrial biogenesis, including, for instance, the amount or expression of mitochondrial proteins. In some further embodiments, the amount or expression of one or more subunits selected from the group of subunits of cytochrome c oxidase, cytochrome c, cytochrome c reductase or ATP synthetase is increased. In some embodiments of any of the above, the radiation is visible or near-infrared radiation.

In other aspects, the invention provides a method of monitoring the effect of treatment with electromagnetic radiation in the visible portion of the spectrum on a mammalian subject, by exposing a tissue of the subject to the radiation and measuring the effect of the radiation on the production of NO on NO-induced vasodilators in the tissue.

In another aspect, the invention provides an in vivo or in vitro method of modulating NO production by cells (e.g., neurons or endothelial cells) in a mammalian tissue capable of producing NO under hypoxic conditions and/or high concentrations of glucose by cyctochrome c nitrite reductase activity, by exposing the neurons or endothelial cells to visible radiation. In these embodiments, a neurodegenerative condition can be treated. In some embodiments, the invention provides methods for increasing NO production and blood flow in the brain tissue of persons having or at increased risk of Alzheimer's disease or another neurodegenerative disease involving altered APP processing and plaque formation. In some embodiments, the invention accordingly provides a method of modulating APP processing or of reducing plaque formation by reducing APP processing in such persons. In some embodiments, visible radiation reverses hypoxia-related or oxidative stress induced APP processing to Aβ1-40 and Aβ1-42, the two major peptides implicated in Alzheimer's. In still other embodiments, the invention further provides methods of phototherapy which enhance or improve cognitive function in Alzheimer's patients.

In any of the above aspects and embodiments, there are further embodiments in which an extremity is irradiated with the electromagnetic radiation in the visible portion of the spectrum. For instance, the extremity in some embodiments is the foot or hand, or lower limb. Also, in any of the above embodiments, there are embodiments in which the tissue can be a tissue of the central nervous system. In some embodiments, the tissue is a brain tissue or spinal cord tissue.

The “electromagnetic radiation in the visible portion of the spectrum” comprises light having wavelengths of about 400 to about 625 nm, 400 to 600 nm, 500 to 650 nm, from 550 to 625 nm, from 575 nm to about 625 nm in wavelength, or from 500 to 600 nm, 550 to 600 nm, and from 575 to 600 nm. In some embodiments, the wavelength of electromagnetic radiation to be used is substantially free of light having a wavelength greater than 600 nm, 610 nm, 615 nm 625 nm, 630 nm, 650 nm, or 675 nm. In some embodiments, the electromagnetic radiation is substantially free of radiation of inhibitory wavelengths of light or is substantially free of light in the 615 to 750 nm range, the 620 to 700 nm range, 630 to 700 nm range, 630 to 750 nm range, 630 to 675 nm range, the 650 and 700 nm range, 625 to 800 nm range. Light which is “substantially free” of certain wavelengths is light which comprises a small proportion (e.g., less that 25%, 20%, 15%, 10%, 5%, or I %) of its total energy at the specified wavelengths) or which has a ratio of light energy in the therapeutic range (e.g., 550 nm to 625 nm) which is at least 3-fold, 4-fold, 5-fold or 10-fold greater than that of those wavelengths which inhibit the effect of the therapeutic light on the mitochondria as measured according to stimulation of NO production under anoxic conditions (e.g., inhibitory wavelengths). In some embodiments, radiation specifically targets the haem absorption bands of cyctochrome c oxidase.

In some embodiments, the wavelength of electromagnetic radiation to be used is principally composed of polychromatic light falling within the above wavelength ranges. By “principally composed’, it is meant that at least 70%, 80%, 90%, or 95% of the energy of the applied light falls within the above wavelength ranges. In some embodiments, the monochromatic or polychromatic electromagnetic radiation is substantially free of radiation having wavelengths in the 615 to 750 nm range, the 620 to 700 nm range, 630 to 700 nm range, 630 to 750 nm range, 630 to 675 nm range, the 650 to 700 nm range, or the 625 to 800 nm range. In further embodiments, the method employ light filters to remove one or more wavelengths of light having a wavelength from 625 to 700 nm from a polychromatic light source before the radiation from the light source is to be applied to the skin. Accordingly, in some embodiments, suitable wavelengths for use according to the invention can principally be composed of 400±25 rim, 500±25 nm, 550±25 nm, and 600±25 or from 375 nm to 625 nm light. In further embodiments of any of the above, the electromagnetic radiation in the visible portion of the spectrum is applied at a level of about 0.5 to 40, 1 to 20, or 2 to 10 joules/cm² per treatment. In some embodiments, the radiation is modulated to provide pulses of the light at a pulse frequency of 4 to 10,000 Hz.

In some embodiments of each of the above aspects and embodiments, the wavelength of visible light has a peak in the transmission spectrum from about 400 to 600 nm, 500 to 650 nm, from 550 to 625 nm, from 575 nm to about 625 nm in wavelength, or from 500 to 600 nm, 550 to 600 nm, from 575 to 600 nm, from 590 to 610 nm, or from 595 to 605 nm. In some further embodiments, the light has a bandwidth of about 10, 20, 25, 30, 40, or 50 nm. In still other embodiments, the wavelength of electromagnetic radiation to be used is principally composed of one or more sources of monochromatic light within the above wavelengths. In other embodiments, the applied light can have a peak in the transmission spectrum of about 590, 591, 592, 593, 594, 595, 596, 597, 598, 599, 600, 601, 602, 603, 604, 605, 606, 607, 608, 609, 610 nm and a bandwidth of from about 5, 10, or 20 nm or less than 5, 10, or 20 nm. The administered light can be continuous or pulsed.

The light source in any of the above embodiments can be a xenon-halogen bulb, a light emitting diode (LED), organic LED, a semiconductor-based light emitting device, or a laser diode.

As known to one of ordinary skill, the dosage regimen for the electromagnetic radiation in the visible portion of the spectrum can be adjusted to fit the individual subject. The period and intensity of treatment can be individualized for each subject and/or tissue. For instance, the frequency, duration, and intensity of the radiation can adjusted according to the severity of the condition, the responsiveness of the patient, and/or according to the thickness and coloration of the skin at the point of exposure. In some embodiments of any of the above aspects, the tissue is irradiated over a treatment period of from 10 sec to 1 hour in length. In some embodiments, the treatment is given once- or twice-a-day; 1-, 2-, 3-, 4-, or 5-times a week, or once- or twice-a month. In some embodiments, the treatment is given once or a few times to treat an acute condition. In other embodiments, the treatment is given on a chronic basis (lasting months to years). In yet other embodiments, the treatment may be intermittent and/or as needed to alleviate the signs and symptoms of the condition to be alleviated. Accordingly, treatments may vary in duration from the acute to the chronic. Additionally, the radiation may be applied internally (e.g., via glass fiber optics) or externally to the tissue or subject. The electromagnetic radiation in the visible portion of the spectrum is preferably not associated with any significant heating of the tissue by the energy of the radiation. In some embodiments, the radiation may be applied locally or proximal to the affected tissue or applied at a location at some distance from the affected tissue to foster a release of NO that acts upon a target tissue at a location not contacted with the applied light.

In yet another aspect, the invention provides a method of prognosis and diagnosis for poor blood circulation or DPN in a tissue or organ by measuring the tissue or blood NO, VEGF, or protein carbonylation levels. In some embodiments, the NO and VEGF levels serve to indicate early stage DPN prior to loss of sensation and pain.

In another aspect, the invention provides a method of monitoring the response to exposure of a tissue to electromagnetic radiation in the visible portion of the spectrum by measuring blood flow in the tissue, or measuring the tissue or blood NO, VEGF, protein carbonylation, nitration, or nitroslylation levels in the blood or tissue. In some embodiments, this aspect can be used in evaluating the response of a tissue or subject exposed to radiation according to any of the other aspects and embodiments of the invention. Accordingly, in some embodiments, the monitoring is used to adjust the radiation treatment regimen for a tissue or subject on either an acute or chronic basis.

In one aspect, the invention provides for the use of electromagnetic radiation in the visible portion of the spectrum in the therapeutic photomodulation of diabetic peripheral neuropathy. Hyperglycemia and endothelial inflammation are thought to promote a series of events that affect the vasculature that may induce DPN. Several recent studies have proposed that reactive oxygen species play a key role in many of these processes and that vascular constriction, reduced blood flow to extremities, hyperglycemia, endoneural hypoxia, nitrosative stress, and oxidative stress may all contribute to the peripheral neuropathies associated with diabetes (Pop-Busai et al. 2006). Methods and instrumentation of providing electromagnetic radiation in the visible portion of the spectrum for use according to the invention (see U.S. patent application Ser. No. 11/331,490, assigned to a same assignee as the present application and incorporated by reference herein its entirety and particularly with respect to such methods and instrumentation) are well known to persons of ordinary skill in the art as are methods of identifying hypoxic tissues, poor blood circulation, hyperglycemia, peripheral neuropathies, and type II diabetes. In some embodiments, light emitting diode (LEDs) arrays or low energy lasers, are contemplated as sources of the radiation. Accordingly, the applied radiation can be coherent or non-coherent.

Accordingly, in a further aspect of any of the above, the invention further provides for a combination therapy comprising use of any one of the above phototherapeutic methods in combination with administration of NO donors and other compounds (substrates for NO synthetase, inhibitors of NO degradative pathways) which modulate NO levels in a subject.

### DEFINITIONS

It is noted here that as used in this specification and the appended claims, the singular forms “a,” “an,” and “the” include plural reference unless the context clearly dictates otherwise. As such, the terms “a” (or “an”), “one or more”, and “at least one” can be used interchangeably herein.

The term “modulate” means to decrease or increase. The modulatory stimulus may be dynamic (varying over time) during application or constant. The visible light modulation of mitochondrial function is illustrated in FIG. 1. The modulation can be therapeutic in nature and result in the treatment (e.g., amelioration, reduction (as to either frequency or severity) or prevention (e.g., delay in on-set or failure to develop) of the recited adverse condition or the signs, symptoms, or adverse sequelae of the recited adverse condition. Modulation can also promote the health of a tissue or subject with respect to a particular condition.

Hypoxia is a condition in which the body as a whole or in part lacks an adequate oxygen supply. The term includes ischemic hypoxia or ischemia as from a restriction in blood supply as may occur in circulatory disorders (e.g., atherosclerosis, macro or microcirculatory disorders) affecting blood flow, edema, or tissue perfusion. Accordingly, cerebral hypoxia refers to a reduced or indequate oxygen supply to brain tissue. Mild or moderate cerebral hypoxia can cause confusion and fainting. In some preferred embodiments, the hypoxia is characterized by increased susceptibility to visible light induced mitochondrial induction of NO production.

The phrase “electromagnetic radiation in the visible portion of the spectrum” comprises light having wavelengths of about 400 to 650 nm, 500 to 650 nm, from 550 to 625 nm, from 575 nm to about 625 nm in wavelength, or from 500 to 600 nm, 550 to 600 nm, from 575 to 600 nm. In some embodiments, the wavelength of electromagnetic radiation to be used is substantially free of light having a wavelength greater than 600 nm, 610 nm, 615 nm 625 nm, 630 nm, 650 nm, or 675 nm. In some embodiments, the electromagnetic radiation is substantially free of radiation of inhibitory wavelengths of light or is substantially free of light in the 615 to 750 nm range, the 620 to 700 nm range, 630 to 700 nm range, 630 to 750 nm range, 630 to 675 nm range, the 650 and 700 nm range, 625 to 800 nm range. Light which is “substantially free” of certain wavelengths is light which comprises a small proportion (e.g., less that 25%, 20%, 15%, 10%, 5%, or 1%) of its total energy at the specified wavelengths) or which has a ratio of light energy in the therapeutic range (e.g., 550 nm to 625 nm) which is at least 3-fold, 4-fold, 5-fold or 10-fold greater than that of those wavelengths which inhibit the effect of the therapeutic light on the mitochondria as measured according to stimulation of NO production under anoxic conditions (e.g., inhibitory wavelengths).

Additionally, the therapeutic radiation can be applied at a level of about 0.5 to 40, 1 to 20, or 2 to 10 joules/cm² per treatment. The radiation can be continuous or pulsed. The radiation can also be modulated to provide pulses of radiation at a pulse frequency of 4 to 10,000 Hz. For instance, in some embodiments, visible radiation is applied as an intensity per treatment of 0.5 to 40 joules/cm² per treatment period and is modulated at a frequency of from 1 to 100 Hz, 4 to 10,000 Hz, 40 to 2000 Hz, 1000 to 5000 Hz, or 100 to 1000 Hz. The treatments can be of varying duration (e.g., ranging from 1 to 5 minutes to an hour or more). For instance, a treatment can last for 5 to 10 minutes, 5 to 20 minutes or 20 to 40 minutes.

Accordingly, the light source used to apply the light preferably generates light in the visible range. In some embodiments of each of the above aspects and embodiments, the wavelength of electromagnetic radiation light to be used comprises wavelengths from about 400 to 625 nm, 400 to 525 nm, 500 to 650 nm, from 550 to 625 nm, from 575 nm to about 625 nm in wavelength, or from 500 to 600 nm, 550 to 600 nm, from 575 to 600 nm. In some embodiments, the wavelength of electromagnetic radiation to be used is substantially free of light having a wavelength greater than 600 nm, 610 nm, 615 nm 625 nm, 630 nm, 650 nm, or 675 nm. In some embodiments, the electromagnetic radiation is substantially free of radiation in the 615 to 750 nm range, the 620 to 700 nm range, 630 to 700 nm range, 630 to 750 nm range, 630 to 675 nm range, the 650 and 700 nm range, or the 625 to 800 nm range.

In certain embodiments, the light source comprises one or more laser diodes, which each provide coherent light. In embodiments in which the light from the light source is coherent, the emitted light may produce “speckling” due to coherent interference of the light. This speckling comprises intensity spikes which are created by constructive interference and can occur in proximity to the target tissue being treated. For example, while the average power density may be approximately 10 mW/cm², the power density of one such intensity spike in proximity to the brain tissue to be treated may be approximately 300 mW/cm². In certain embodiments, this increased power density due to speckling can improve the efficacy of treatments using coherent light over those using incoherent light for illumination of deeper tissues.

In other embodiments, the light source provides incoherent light. Exemplary light sources of incoherent light include, but are not limited to, incandescent lamps or light-emitting diodes. A heat sink can be used with the light source (for either coherent or incoherent sources) to remove heat from the light source and to inhibit temperature increases at the scalp. In certain embodiments, the light source generates light which is substantially monochromatic (i.e., light having one wavelength, or light having a narrow band of wavelengths).

In further embodiments of the above, the light source generates or provides light having a plurality of wavelengths, but with the proviso that the light is substantially free of light having wavelengths ranging from 650 to 750 nm. In some embodiments, one or more optical filters are used to remove a portion of light having a wavelength falling between 625 and 750 nm.

The light source is capable of emitting light energy at a power sufficient to achieve a predetermined power density at the subdermal target tissue (e.g., at a depth of approximately 2 centimeters from the dura with respect to the brain). It is presently believed that phototherapy of tissue is most effective when irradiating the target tissue with power densities of light of at least about 0.01 mW/cm² and up to about 1 W/cm². In various embodiments, the subsurface power density is at least about 0.01, 0.05, 0.1, 0.5, 1, 5, 10, 15, 20, 30, 40, 50, 60, 70, 80, or 90 mW/cm², respectively, depending on the desired clinical performance. In certain embodiments, the subsurface power density is preferably about 0.01 mW/cm² to about 100 mW/cm², more preferably about 0.01 mW/cm² to about 50 mW/cm², and most preferably about 2 mW/cm² to about 20 mW/cm². It is believed that these subsurface power densities are especially effective at producing the desired biostimulative effects on the tissue being treated. Taking into account the attenuation of energy as it propagates from the skin surface, through body tissue, bone, and fluids, to the subdermal target tissue, surface power densities preferably between about 10 mW/cm² to about 10 W/cm², or more preferably between about 100 mW/cm² to about 500 mW/cm², can typically be used to attain the selected power densities at the subdermal target tissue. To achieve such surface power densities, the light source is preferably capable of emitting light energy having a total power output of at least about 25 mW to about 100 W. In various embodiments, the total power output is limited to be no more than about 30, 50, 75, 100, 150, 200, 250, 300, 400, or 500 mW, respectively. In certain embodiments, the light source comprises a plurality of sources used in combination to provide the total power output. The actual power output of the light source is preferably controllably variable. In this way, the power of the light energy emitted can be adjusted in accordance with a selected power density at the subdermal tissue being treated.

Certain embodiments utilize a light source that includes only a single laser diode that is capable of providing about 10, 20, 25, 30, 40, or 50 mW to about 100 W of total power output at the skin surface. In certain such embodiments, the laser diode can be optically coupled to the scalp via an optical fiber or can be configured to provide a sufficiently large spot size to avoid power densities which would burn or otherwise damage the skin. In other embodiments, the light source utilizes a plurality of sources (e.g., laser diodes) arranged in a grid or array that together are capable of providing at least 10, 20, 25, 30, 40, or 50 mW to about 100 W of total power output at the skin surface. The light source of other embodiments may also comprise sources having power capacities outside of these limits.

In certain embodiments, the light source generates light which cause eye damage if viewed by an individual. In such embodiments, the light source apparatus can be configured to provide eye protection so as to avoid viewing of the light by individuals. For example, opaque materials can be appropriately placed to block the light from being viewed directly. In addition, interlocks can be provided so that the light source apparatus is not activated unless the protective element are in place, or other appropriate safety measures are taken.

In still other embodiments, the therapy apparatus for delivering the light energy includes a handheld probe.

In certain embodiments, the application of the light is controlled programmable controller comprising a logic circuit, a clock coupled to the logic circuit, and an interface coupled to the logic circuit. The clock of certain embodiments provides a timing signal to the logic circuit so that the logic circuit can monitor and control timing intervals of the applied light. Examples of timing intervals include, but are not limited to, total treatment times, pulse width times for pulses of applied light, and time intervals between pulses of applied light. In certain embodiments, the light sources can be selectively turned on and off to reduce the thermal load on the skin and to deliver a selected power density to particular areas of the brain or other target tissue/organ.

In some embodiments, the applied light source is controlled by a logic circuit coupled to an interface. The interface can comprise a user interface or an interface to a sensor monitoring at least one parameter of the treatment. In certain such embodiments, the programmable controller is responsive to signals from the sensor to preferably adjust the treatment parameters to optimize the measured response. The programmable controller can thus provide closed-loop monitoring and adjustment of various treatment parameters to optimize the phototherapy. The signals provided by the interface from a user are indicative of parameters that may include, but are not limited to, patient characteristics (e.g., skin type, fat percentage), selected applied power densities, target time intervals, and power density/timing profiles for the applied light.

In certain embodiments, the logic circuit is coupled to a light source driver. The light source driver is coupled to a power supply, which in certain embodiments comprises a battery and in other embodiments comprises an alternating current source. The light source driver is also coupled to the light source. The logic circuit is responsive to the signal from the clock and to user input from the user interface to transmit a control signal to the light source driver. In response to the control signal from the logic circuit, the light source driver adjust and controls the power applied to the light sources.

In certain embodiments, the logic circuit is responsive to signals from a sensor monitoring at least one parameter of the treatment to control the applied light. For example, certain embodiments comprise a temperature sensor thermally coupled to the skin to provide information regarding the temperature of the skin to the logic circuit. In such embodiments, the logic circuit is responsive to the information from the temperature sensor to transmit a control signal to the light source driver so as to adjust the parameters of the applied light to maintain the scalp temperature below a predetermined level. Other embodiments include exemplary biomedical sensors including, but not limited to, a blood flow sensor, a blood gas (e.g., oxygenation) sensor, an NO production sensor, or a cellular activity sensor. Such biomedical sensors can provide real-time feedback information to the logic circuit. In certain such embodiments, the logic circuit is responsive to signals from the sensors to preferably adjust the parameters of the applied light to optimize the measured response. The logic circuit can thus provide closed-loop monitoring and adjustment of various parameters of the applied light to optimize the phototherapy.

Preferred methods of phototherapy for a selected wavelength(s) are based upon recognition that the power density (light intensity or power per unit area, in W/cm²) or the energy density (energy per unit area, in J/cm², or power density multiplied by the exposure time) of the light energy delivered to tissue is an important factor in determining the relative efficacy of the phototherapy.

In certain embodiments, the light source can be adjusted to irradiate different portions of the subject's skin or scalp in order to target underlying brain tissue which, or instance, has been the subject of a pathology or neurodegeneration.

As used herein, the term “neurodegeneration” refers to the process of cell destruction or loss of function resulting from primary destructive events such as stroke or CVA, as well as from secondary, delayed and progressive destructive mechanisms that are invoked by cells due to the occurrence of the primary destructive event. Primary destructive events include disease processes or physical injury or insult, including stroke, but also include other diseases and conditions such as multiple sclerosis, amyotrophic lateral sclerosis, epilepsy, Alzheimer's disease, dementia resulting from other causes such as AIDS, cerebral ischemia including focal cerebral ischemia, and physical trauma such as crush or compression injury in the CNS, including a crush or compression injury of the brain, spinal cord, nerves or retina, or any acute injury or insult producing neurodegeneration. In some embodiments, the methods according to the invention can be used to treat Huntington disease; Parkinson disease; familial Parkinson disease; Alzheimer disease; familial Alzheimer disease; amyotrophic lateral sclerosis; sporadic amyotrophic lateral sclerosis; mitochondrial encephalomyopathy with lactic acidosis and strokelike episodes; myoclonus epilepsy with ragged-red fibers; Kearns-Sayre syndrome; progressive external ophthalmoplegia; Leber hereditary optic neuropathy (LHON); Leigh syndrome; and Friedreich ataxia, and cytochrome c oxidase (Cco) deficiency states.

As used herein, the term “neuroprotection” refers to a therapeutic strategy for slowing or preventing the otherwise irreversible loss of neurons or CNS function due to neurodegeneration after a primary destructive event, whether the neurodegeneration loss is due to disease mechanisms associated with the primary destructive event or secondary destructive mechanisms.

Additionally, inflammation and oxidative stress are important in the pathology of many chronic neurodegenerative conditions, including Alzheimer's disease. This disease is characterized by the accumulation of neurofibrillary tangles and senile plaques, and a widespread progressive degeneration of neurons in brain. Senile plaques are rich in amyloid precursor protein (APP) that is encoded by the APP gene located on chromosome 21. Pathogenesis of AD may be mediated by an abnormal proteolytic cleavage of APP which leads to an excess extracellular accumulation of beta-amyloid peptide which is toxic to neurons (Selkoe et al., (1996), J. Biol. Chem. 271:487-498; Quinn et al., (2001), Exp. Neurol. 168:203-212; Mattson et al., (1997), Alzheimer's Dis. Rev. 12:1-14; and Fakuyama et al., (1994), Brain Res. 667:269-272). Methods of assessing neuroprotection are well known in the art (see, for instance, U.S. Patent publication no. 20080107603 and U.S. Pat. No. 6,803,233 which are incorporated herein by reference). A beneficial outcome of light dependent Cco NO production is the nitrosylation and subsequent down regulation of gamma secretase activity. The decreased gamma secretase activity would in turn decrease the production of harmful beta amyloid peptides.

Accordingly, without being wed to theory, in some embodiments, an object of the present invention is to provide a treatment of dementia which can ameliorating learning and/or memory impairments, or cognitive impairment in Alzheimer-type dementia, cerebrovascular dementia and senile dementia.

In some embodiments, the invention provides a method of treating a subject having a disorder involving impaired mitochondrial function. Generally, the method includes administering a phototherapy of the present invention to such a subject under conditions effective to improve mitochondrial function. This method of the present invention is particularly useful for the treatment or prophylaxis of disorders associated with impaired mitochondrial function. Disorders that can be treated according to this method generally include conditions or diseases characterized by a decreased level of oxidative metabolism. The disorders may be caused by genetic factors, environmental factors, or both. More specifically, such disorders include conditions or diseases of the nervous system (e.g., neurodegenerative, psychoses, etc.), conditions or diseases of other parts of the body, and conditions or diseases of the body as a whole. Such conditions or diseases of the nervous system include not only Alzheimer's Disease, Parkinson's Disease, Huntington's Disease, but also spinocerebellar ataxias, and psychoses (including depression or schizophrenia) associated with oxidative metabolic abnormalities. Exemplary conditions or disorders of other parts of the body include cardiovascular disorders (e.g., atherosclerotic and cardiovascular diseases including myocardial infarctions, angina, cardiomyopathies, cardiac valvular disorders, and other conditions or disorders causing cardiac failure), musculoskeletal disorders in which oxidative metabolism is abnormal and other conditions or disorders of non-neural tissues in which oxidative metabolism is abnormal, such as frailty, a geriatric syndrome often associated with metabolic alterations.

Many conditions or diseases of the nervous system (e.g., AD and those described above) are characterized by cerebral metabolic insufficiencies, which are manifested as impaired cerebral function such as dementia. Therefore, another aspect of the present invention relates to a method of improving cerebral function in a subject having cerebral metabolic insufficiencies. Generally, a treatment of the present invention is administered to a subject having impaired cerebral metabolism under conditions effective to improve the cerebral cellular metabolism. By improving cerebral cellular metabolism, the subject's cerebral function is improved significantly

The terms “treating” or “treatment” refer to therapeutic methods involving the application of an agent which benefits a particular disease or condition. For instance, a phototherapy according to the invention can be used to slow the progression or onset of the disease or condition, and/or to reduce the signs and/or symptoms or physical manifestations of the disease or condition. A therapeutically effective amount of an agent references a quantity or dose of an agent (e.g., radiation or drug) which is sufficient to treat the disease or condition. Many models systems for determining the efficacy of neuroprotective agents are known in the art. Such model systems can be used to assess the efficacy of treatments according to the invention. For instance, behavioral assessments as known to one of ordinary skill in the art can be used in humans or test animals for cognitive impairment. In test animals, the spatial memory test using Y-maze apparatus can be used test the behavioral property of animals to enter into a new arm, avoiding the arm that they entered into just before (alternation behavior). (see, Itoh, J. et al. (Eur. J. Pharmacol., 236, 341-345 (1993)). Alternatively or additionally, histopathological methods monitoring cell death, accumulation of neurofibrillary tangles or senile plaque can be used to assess the extent of neurodegeneration.

A neuroprotective-effective amount of light energy achieves the goal of reversing, preventing, avoiding, reducing, or eliminating neurodegeneration.

In certain embodiments, the “neuroprotection” involves treating a patient (e.g., Alzheimer's disease) by placing the therapy apparatus in contact with the scalp and adjacent the target area of the patient's brain. The target area of the patient's brain can be previously identified such as by using standard medical imaging techniques. In certain embodiments, treatment further includes calculating a surface power density at the scalp which corresponds to a preselected power density at the target area of the patient's brain. The calculation of certain embodiments includes factors that affect the penetration of the light energy and thus the power density at the target area. These factors include, but are not limited to, the thickness of the patient's skull, type of hair and hair coloration, skin coloration and pigmentation, patient's age, patient's gender, and the distance to the target area within the brain. The power density and other parameters of the applied light are then adjusted according to the results of the calculation.

The power density selected to be applied to the target area of the patient's brain depends on a number of factors, including, but not limited to, the wavelength of the applied light, the location and severity of the pathology, and the patient's clinical condition, including the extent of the affected brain area. The power density of light energy to be delivered to the target area of the patient's brain may also be adjusted to be combined with any other therapeutic agent or agents, especially pharmaceutical neuroprotective agents, to achieve the desired biological effect. In such embodiments, the selected power density can also depend on the additional therapeutic agent or agents chosen.

In preferred embodiments, the treatment proceeds continuously for a period of about 10 seconds to about 2 hours, more preferably for a period of about 1 to about 10 minutes, and most preferably for a period of about 2 to 5 minutes. In other embodiments, the light energy is preferably delivered for at least one treatment period of at least about five minutes, and more preferably for at least one treatment period of at least ten minutes. The light energy can be pulsed during the treatment period or the light energy can be continuously applied during the treatment period.

In some embodiments, the light is delivered at a rate of from about 0.5 to 8, 2 to 6, or about 4 mW/cm² on average to a target site over a given treatment duration. In some further embodiments, the treatment is for about 0.1, 0.2, 0.4, 0.8, 1, 2, 3, 4, 5, 6, or 8 hours. In some embodiments, the light is provided in a total amount from 0.5 to 40 joules/cm²/treatment.

In certain embodiments, the treatment may be terminated after one treatment period, while in other embodiments, the treatment may be repeated for at least two treatment periods. The time between subsequent treatment periods is preferably at least about five minutes, more preferably at least about 1 to 2 days, and most preferably at least about one week. The length of treatment time and frequency of treatment periods can depend on several factors, including the functional recovery or response of the patient to the therapy.

A method for the neuroprotective treatment of a patient in need of such treatment involves delivering a neuroprotective-effective amount of light energy having a wavelength in the visible range to a target area of the patient's brain. In certain embodiments, the target area of the patient's brain includes the area of plaque accumulation or ischemia, i.e., to neurons within the “zone of danger.” In other embodiments, the target area includes portions of the brain not within the zone of danger. Without being bound by theory, it is believed that irradiation of healthy tissue in proximity to the zone of danger increases the production of NO in the irradiated tissue, which can improve blood flow in adjoining hypoxic tissue, including injured tissue.

Apparatus and methods adaptable for in the application of light to the brain according to the present invention are disclosed in U.S. Patent Application Publication No. 2006/0253177 which is incorporated herein by reference.

In certain embodiments, a method provides a neuroprotective effect in a patient that has had an ischemic event in the brain. The method comprises identifying a patient who has experienced an ischemic event in the brain. The method further comprises estimating the time of the ischemic event. The method further comprises commencing administration of a neuroprotective effective amount of light energy to the brain or the affected area of the brain and/or an area proximal thereto. The administration of the light energy is commenced no less than about two hours following the time of the ischemic event. In certain embodiments, phototherapy treatment can be efficaciously performed preferably within 24 hours after the ischemic event occurs, and more preferably no earlier than two hours following the ischemic event, still more preferably no earlier than three hours following the ischemic event, and most preferably no earlier than five hours following the ischemic event. In certain embodiments, one or more of the treatment parameters can be varied depending on the amount of time that has elapsed since an ischemic event.

Certain embodiments also provide a method of treating Alzheimer's disease (e.g., slowing the progression or onset of the condition, or reducing the signs and/or symptoms or physical manifestations of the disease). Much evidence indicates that less oxygenated blood flowing to the brain contributes to the build-up of the protein plaques associated with Alzheimer's disease. Alterations in mitochondrial function, including particularly cytochrome c-oxidase activity have also been reported in Alzheimer's disease patients as well. We have shown that under hypoxic conditions, that visible light can activate cytochrome-c to produce nitric oxide, a potent vasodilator. Vasodilation can increase the amount oxygen available to cells as well as directly promote mitochondrial function in these patients. Accordingly, in one aspect, the invention provides phototherapy for Alzheimer's disease.

In some embodiments of the above where the brain is to be treated, the external carotid artery and or the vertebral artery are exposed to light by the application of the visible light from the sides of the head (e.g., the temples). The shortest distance to these structures is from the sides. Positioning the treatment heads or light sources directly under the ear and behind the jaw bone would give the most direct access to these structures for the radiant energy applied. In other embodiments, the vertebral artery is treated by the application of the light to the from the rear of the skull or from the sides of the skull. When the brain or a head tissue is to be treated, the light can be applied to any portion of the skull, including the forehead and any combination of the above, especially when close to the affected or target site for application of the light.

In some embodiments where the brain is to be treated, the treatment head should be applied to the below the ear and just behind the jaw bone (see, FIG. 9). This will maximize the irradiation area of the supplying vessels to the brain due to having to traverse less soft tissue. This illustrates that a treatment head of approximately 2″ inches in diameter would cover both structures. Other treatment heads may be used, including those of from 0.5 to 4 inches in diameter can be used. The treatment heads need not be circular but can be configured so as to track the location of the targeted arteries more closely. In some embodiments, the treatment heads can have an application surface area of from about one or two square inches to 4, 8 or 10 square inches. In some embodiments, the treatments can be applied to either or both sides of the body.

In some embodiments, the methods of treatment may use a light emitting diode (LED), an organic LED (OLED) device formulated into a single-use or multiple use patch to provide the electromagnetic radiation or visible light for use according to the invention, or another semiconductor-based light emitting device. In a single use format, a battery having sufficient power for only a single-use treatment would be incorporated into the patch thereby producing a unit dose of the therapeutic light. For single or multiple uses, other sources of power could be utilized as known to persons of skill in the art. An exemplary patch or bandage may include materials such as textile fabrics, tapes, or films. For example, a patch may include natural or synthetic rubber, fabric, cotton, polyester, and the like. Optionally, a patch may include an adhesive substance, glue, bonding agent, strap, tie, fastener, or means of fixing the LED or OLED on the patient. In some cases, a patch may incorporate a natural or synthetic adhesive, a medical adhesive, or a bioadhesive. An adhesive can be coated on a surface of a patch, or impregnated within a dressing or matrix of a patch. In some cases, a patch may include a curable gel or paste. A patch can serve to secure placement of an LED or OLED at a desired location on a patient. A patch may be flexible or curved, so as to conform with any of a variety of surface shapes. A patch may include one or more layers of material. The patch may be configured so as to maximize or modulate the amount of electromagnetic radiation incident upon the patient.

Accordingly, it is contemplated, that some embodiments of a photobiomodulation medical device may take the form of a multi- or single-use LED or Organic light emitting diode (OLED) patch. The patch may be charged with a unit dose of energy capable of delivering from 0.5 to 8 mW/cm² on average to a target site over a treatment duration. The light can be continuous or pulsatile as described above. Without being wed to theory, the patch when used in therapy for Alzheimer's disease patients may be applied either to the neck or head of the patient:
Endothelial Targeting: By placing the LED or/and patch over the carotid artery of the neck, and targeting CCO in the endothelial cells and/or smooth muscle cells lining the artery, circulation and oxygenation to the brain is increased, thereby reducing oxidative stress, the accumulation of amyloid peptides and slowing or reversing the progression of the disease.
Neuronal Targeting: By placing the CLARIMEDIX patch on the head, targeting CCO in neurons within the brain, NO production is increased, to affect gamma secretase activity, causing abeta 40 and 42 to be reduced back to normal levels and slowing or reversing progression of the disease.

OLED devices are well known in the technological arts. In general, an organic LED consists of an anode into which current (hole current specifically) is injected, a hole transport layer (that often also serves as an electron blocking layer), then an active medium in which recombination takes place with the electron current that is injected from the cathode through an electron transport medium (that often serves as a hole blocking medium). The active medium consists of efficient recombination centers if the device is to function with high luminescent efficiency. In a small molecule organic structure, each of these layers are well defined, and in fact, the small molecule organic devices often try to mimic the operation of inorganic devices despite the significantly lower conductivity of the organic conductors. In the above sense, as was pointed out by Yu and Heeger (Yu and Heeger, 1995), polymer devices are much superior to either inorganic or small molecule devices. This is because the long polymer strands can overlap and make boundaries indistinct. This effect is equivalent to distributing the junctions between the materials throughout the device volume thereby increasing efficiency. A further important practical advantage to OLED devices is the ability to be fabricated on flexible substrates (see below). Such advantages are important when considering the ultimate use in a biomedical patch and because of the advanced stage of OLED fabrication such devices are particular suitable candidates for a photobiomodulation device. Non-limiting examples of an OLED stack device, an OLED energy diagram, a generic fabrication structure of OLEDs, and materials that may be used in OLEDS are shown in FIG. 32.

Organic light emitting diodes on flexible substrates for patch application to the head and neck are also contemplated as sources of the light to be administered. For example, these OLEDS can have a peak luminescence, for instance, of 400, 550, 600 and 650 nm and can produce output powers on the order of 1 mW for dosage periods, for instance, of seconds to minutes and possess lifetimes of up to hundreds of hours. In some embodiments, the OLED provides a light intensity of 1 to 8 mW/cm² is applied to a target site over a treatment duration. The light can be continuous or pulsatile as described above.

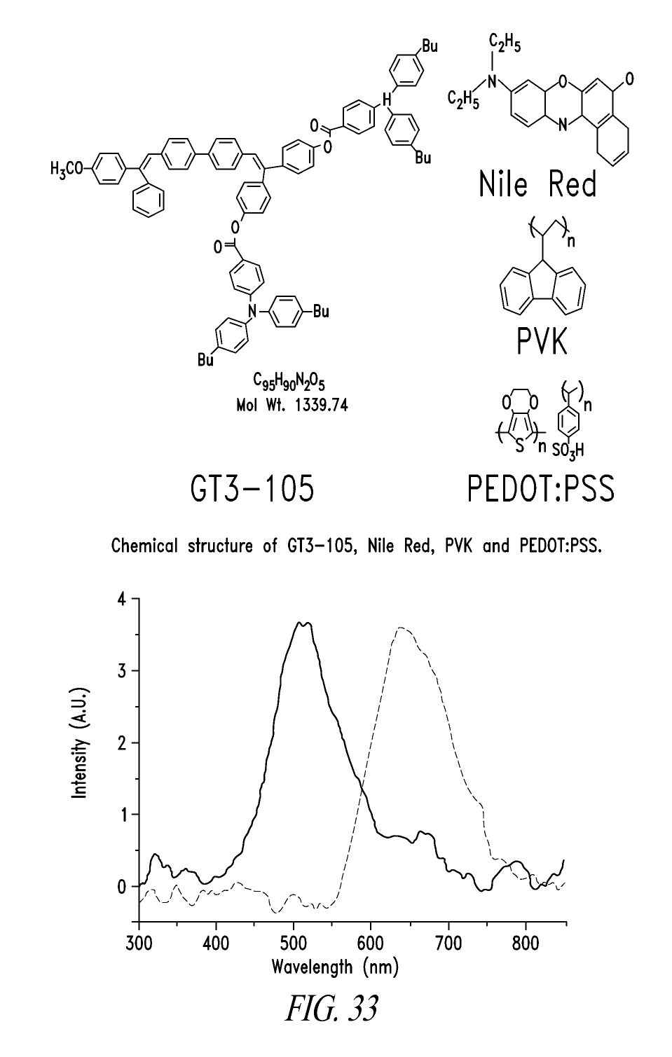

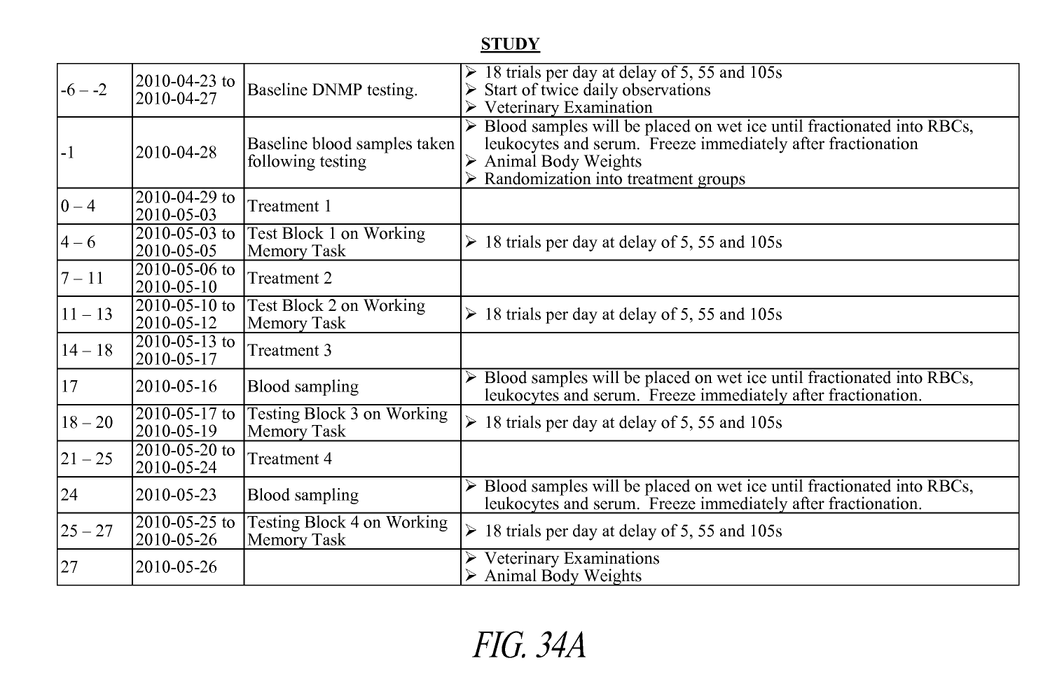

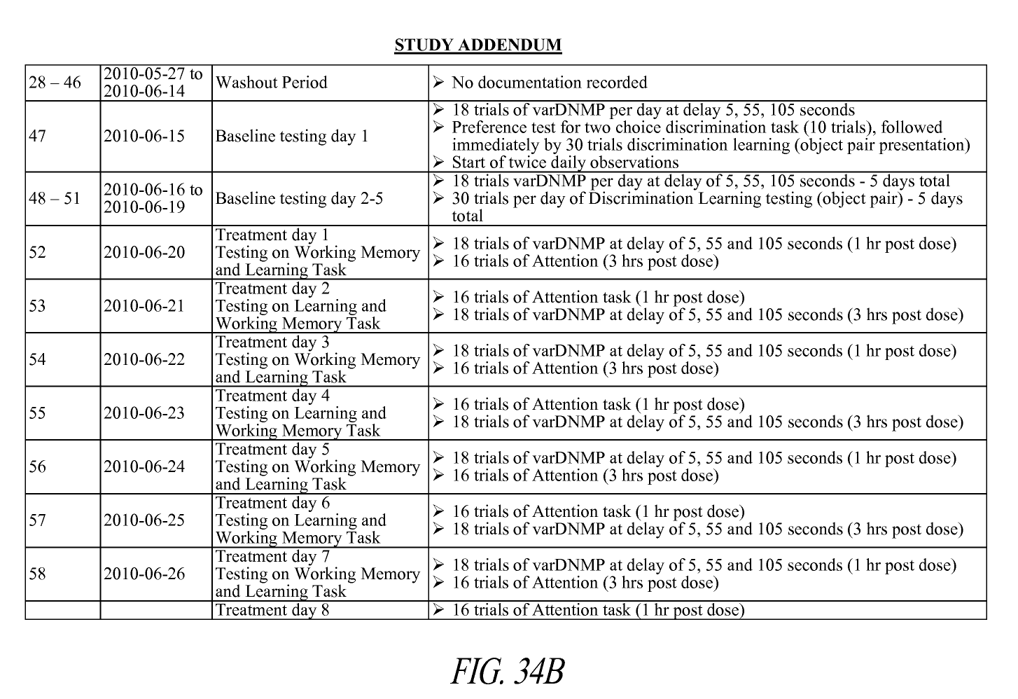

Choice of Dye Layer material. An OLED design that exploits the distribution of the various junctions (electron transport to recombination, hole transport to recombination, hole injection to hole transport, electron injection to electron transport) is given in (Wu et al. 1997). Polyvinylcarbazole (PVK) is a hole transport medium that can also serve as a radiator (recombination region). The recombination efficiency (luminescent efficiency) of PVK is not especially high. Recombination on certain dyes (for example, certain laser dyes in particular) can be quite high. For example, FIG. 33 illustrates the stereo chemical formulae of two different dye compounds, GT3-105 (as named by Lumera Incorporated) and Nile Red. This figure illustrates the luminescence first of GT3-105 (in the blue) and then of Nile Red doped into PVK. Although a priori the PVK exhibits electroluminescence itself, an interesting paper (Pschenitzka and Sturm, 2001) describes a set of experiments that show that co-dopants generally do not all radiate but rather there is a winner-take-all effect in which only the most efficient radiator radiates and that this radiator consumes all of the inversion that the radiator can. Thus one can produce mixed transport media and dye to achieve maximal efficiency and optimal wavelength emission. The distribution of interfaces goes beyond just this level, though. In the design of (Wu et al. 1997), the electron transport, hole transport and recombination dyes are all doped together in the PVK. The efficiency of the resulting devices that use a number of different dyes in order to achieve different wavelength operation, are all much more efficient radiators than the initial blue luminescent PVK.

Choice of flexible substrate materials. OLEDs can be fabricated on a flexible substrates consisting of an acrylate coated with thin enough layers of indium tin oxide (ITO) such that the brittleness of tin oxide does not affect the flexibility of the overall structure. The ITO material is both transparent in the visible and near infrared and is also a good electrical conductor. ITO in combination with polyethylenenedioxythiophene (PEDOT) that is often doped with polystyrenesulfonate (PSS) is a very efficient hole conduction injection combination due to the relative locations of the lowest unoccupied molecular orbital (LUMOs) and the energy gap between this level and the highest occupied molecular orbital (HOMOs). By using the PEDOT/PSS coated ITO as an anode and a calcium surface treated upper aluminum electrode as cathode, efficient injection of holes and electrons into the flexible polymeric material can be achieved. This design integrates the flexible substrate and the OLED together to achieve a novel integrated device. The fabrication of OLEDs follows general procedures. Pilot fabrication runs will be performed to determine optimal layer thickness, curing times, and the like. In some embodiments, OLED devices having emission wavelengths of 400, 500, 550, 600 and 650 nm, with conversation efficiencies of >15%, and lifetimes of great than 100 hours are contemplated.

In some further embodiments, the OLED patch device may be used according to emit light of a suitable wavelength to treat Alzheimer's disease in the methods according to the invention and/or to improve cerebral blood flow. The device can serve to stimulate the production of NO by endothelial cells that line the vascular system. This NO production in turn, improves circulation and oxygenation in and to the brain, thereby preventing or reducing the low oxygen conditions that can contribute to neurological impairment, including, in the case of Alzheimer's disease, the formation of Alzheimer's plaques and tangles. The ability of NIR light to penetrate the scalp and skull to reach underlying tissue is evidenced by Quan Zhang et al., Journal of Biomedical Optics 5(2), 206-213 (April 2000).

The device is also targeting neurons directly, the technology by increasing nitric oxide production within the exposed neurons thereby reducing or preventing harmful APP processing.

In some embodiments, the effect of the treatment on Alzheimer's disease can be monitored by assessing the effect on the treatment on the disease progression itself or indirectly by monitoring biomarkers of disease progression or pathogenesis (e.g., APP and APP products, gamma secretase (including but not limited to particularly the presenilin subunit) levels; mitochondrial Cco subunit IV (mammalian, V yeast) isoforms)). (see, Schon et al., J. Clin. Invest. 111(3): 303-312 (2003)).

### Example 1

### Role of the Respiratory Chain in No Production in Endothelial Cells Under Hypoxic Conditions

Currently, there are two known pathways for NO synthesis. The first involves nitric oxide synthase (NOS), an enzyme that converts arginine to citrulline in the presence of NADPH and oxygen. There are three isoforms of nitric oxide syththase (NOS). These are designated NOS I (neuronal NOS), NOS II (inducible NOS), and NOS III (endothelial NOS). The second pathway for NO production involves nitrite-dependent NO production by the mitochondrial respiratory chain. This pathway is active only at reduced oxygen concentrations.

The relative importance of the NOS-dependent and NOS-independent NO synthesis in endothelial cells is assessed before and after visible light treatment. The production of NO is evaluated in cells exposed to hypoxic conditions in the presence of physiological concentration of nitrite. The involvement of the respiratory chain in this process is evaluated in the presence of: a) L-NAME, a general NOS inhibitor, b) inhibitors of the respiratory chain, c) disruptors of the mitochondrial membrane potential, d) inhibitors of mitochondrial complex IV, e) inhibitors of constitutive NOS, and e) theophylline.

### Example 2

### NO Production by Endothelial Cells

Endothelial cells are isolated and cultured as described elsewhere (Wang et al., 2007; Wang et al., 2004). Hypoxia (1.5% 0₂, 93.5% N₂, 5% CO₂) or anoxia (5% CO₂, 4% H₂, 91% N₂) is established in an IN VIVO workstation (Biotrace) or Coy laboratories glove box, pre-equilibrated with the appropriate gas mixture. All cell extracts are prepared inside the workstation or glove box to prevent re-oxygenation. Cells are maintained under anoxic or hypoxic conditions for varying lengths of time (2-8 hr). Nitric oxide production is evaluated with the fluorescent nitric oxide indicator DAF-FM (Molecular Probes, CA). Nutrient media are supplemented with 20 gM NₐNO2. The involvement of the respiratory chain in nitrite dependent NO production is evaluated in the presence of: a) the inhibitors of complex III Antimycin A (10 μM), myxothiazol (10 μM) and Cyanide (1 mM); b) disruptors of the mitochondrial membrane potential FCCP (10 μM) and dinitrophenol (100 μM) c) inhibitors of mitochondrial complex V oligomycin 10 μM, and d) L-NAME, an inhibitor of constitutive NOS L-NAME (1 mM).

### Example 3

### Mitochondrial Functionality and No Production

Mitochondria from normal and hypoxic cells is isolated and evaluated for respiratory control, hypoxic production of nitrite dependent NO production, and production of nitrite dependent NO production after incubation with ATP and theophylline, using methods described previously (Castello et al., 2006).

### Example 4

### Stimulation of Nitrite Reductase Activity and Subunit Phosphorylation of Cytochrome C Oxidase by Visible Light

The effects of visible light on the nitrite-reductase activity of cytochrome c oxidase can be assessed in isolated mitochondria and purified cytochrome c oxidase.

Nitrite-dependent NO production. Initially, NO levels are measured in isolated mitochondria, using an NO meter or the fluorescent probe DAF-FM (Molecular Probes, CA). Mitochondria exposed to visible light are treated with specific respiratory inhibitors in order to localize NO production to cytochrome c oxidase, as described previously (Castello et al. 2006). Visible light stimulation of NO production in mitochondria is observed.

The effects of visible light on nitrite-dependent NO production by isolated cytochrome c oxidase, purified from both mammals and yeast is next studied.

Phosphorylation of subunits of cytochrome c oxidase. The Tyr-phosphorylation of COX is analyzed following immunoprecipitation, gel electrophoresis, and immunoblotting (Lee et al., 2005)

### Example 5

### Effect of Visible Light on Intracellular Levels of Oxidative Stress and/or Mitochondrial Biogenesis in Endothelial and Yeast Cells

These studies examine the long-term effects of visible exposure on endothelial and yeast cells in culture. Specifically, visible exposure is found to enhance the production of new mitochondria and an increase in cellular respiratory metabolism. This result is shown by assessing the effects of visible light on cellular respiration and the intracellular levels of mitochondrial proteins, including the subunits of cytochrome c oxidase. Increased rates of cellular respiration lead to reduced generation of ROS by the mitochondrial respiratory chain. The effects of visible light on cellular respiration, oxidative stress, and mitochondrial biogenesis. (i.e., the synthesis of new mitochondria) are evaluated. By changing the wavelength(s) of visible radiation used to expose the wavelengths most effective for treating hypoxia are identified.

### Example 6

### Mitochondrial Hydrogen Peroxide Production

One way of assessing the effects of visible light on cellular oxidative stress is to measure the production of ROS by the respiratory chain. This is done using isolated mitochondria and an hydrogen peroxide electrode connected to a W.P.I. amplifier.

Measurement of protein carbonylation. Generally speaking, three types of assays are used for assessing cellular oxidative stress. The first makes use of fluorescent dyes (e.g., derivatives of fluorescein or rhodamine) to estimate intracellular ROS levels. The second assesses oxidative damage, caused by ROS, by measuring the accumulation of lipid peroxides (e.g., malonaldehyde and hydroxyalkenals), oxidized nucleosides (e.g., 8-hydroxy-2′-deoxyguanosine (8OH2gG), or oxidized amino acid side chains on proteins (e.g., o-tyrosine, m-tyrosine, dityrosine, and carbonyl derivatives). The third measures the expression of oxidative stress-induced genes.

Protein carbonylation is used to indicate overall levels of cellular oxidative stress. Carbonyl content of mitochondrial and cytosolic protein fractions is measured after derivatizing proteins in each fraction with 2,4-dinitrophenyl hydrazine (DNPH) as described (Dirmeier et al. 2002; 2004).

Mitochondrial biogenesis. In order to determine if light impacts the synthesis of new mitochondria and, consequently, the level of cellular respiration, oxygen consumption rates are measured using an oxygen electrode. Altered intracellular levels of key mitochondrial proteins (subunits of cytochrome c oxidase, cytochrome c, cytochrome c reductase, and ATP synthase) are measured after cells are exposed to light. Levels of these proteins are determined by immunoblotting SDS-gels of whole cell extracts.

### Example 7

### Visible Light Increases Levels of Vasodilators in the Blood

NO and VEGF are measured in venous blood and exposed tissues after patients with peripheral neuropathies are exposed to visible light. VEGF levels will be assessed by an immunoassay and NO levels will be measured with an NO meter or the fluorescent NO indicator DAF-FM.

Measurement of NO levels in the blood. Venous blood will be collected from patients and frozen. Because NO is unstable and rapidly converted to nitrate in the presence of oxidized hemoglobin one will not be able to measure NO directly. Instead, one converts nitrate to nitrite and NO chemically, using copper-coated cadmium as a reducer (NITRALYZER™-II, WPI, FL). The NO that is produced can be measured with an NO electrode connected to a NO/Free radical analyzer.

VEGF levels. VEGF levels in the blood will be determined after running whole blood on an SDS-polyacrylamide gel and immunoblotting the gel with an antibody specific for VEGF.

### Example 8

### The Relationship Between Light and Cellular Nitrite-Dependent Nitric Oxide Production

The overall goal of this study was to examine the relationship(s) between light and cellular nitrite-dependent nitric oxide production by mitochondrial cytochrome c oxidase. The yeast Saccharomyces cerevisiae was used as a model for these studies. Specific Aims were to:

1) Determine if light affects nitrite-dependent nitric oxide production in yeast cells and if so, assess whether it has a stimulatory or inhibitory effect.

2) Determine the effects of light intensity on cellular nitrite-dependent nitric oxide production.

3) Identify an action spectrum for the stimulatory or inhibitory effects of light on cellular nitrite-dependent nitric oxide production.

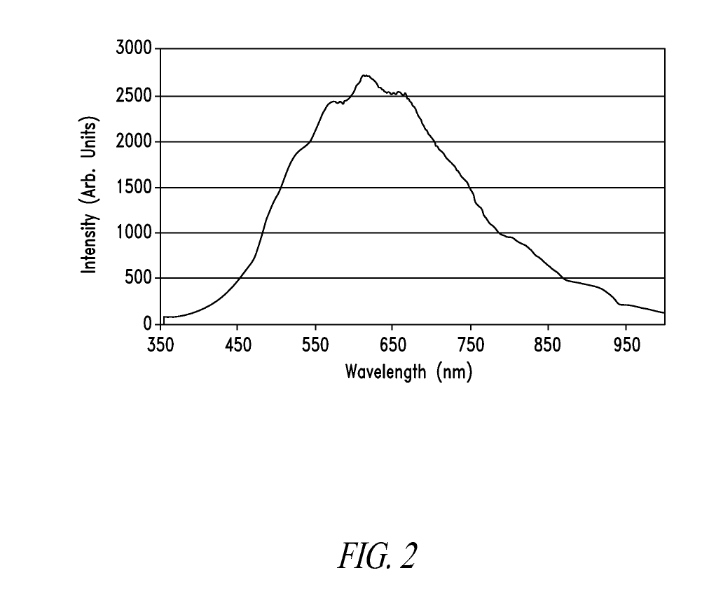

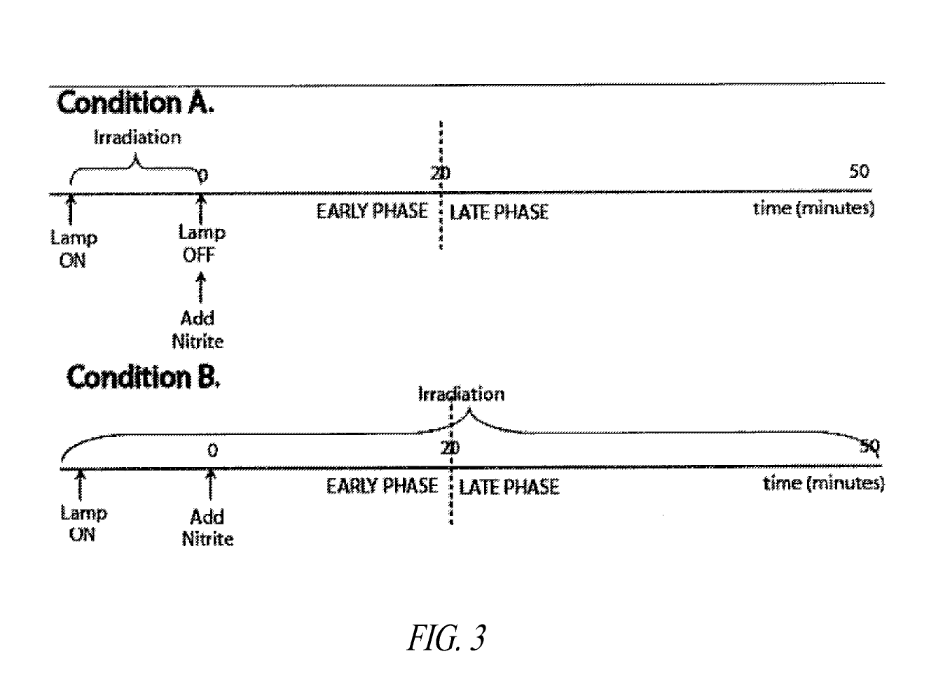

The effects of broad spectrum light on nitrite-dependent nitric oxide production by hypoxic yeast cells was examined. Initially, several experimental conditions were surveyed in order to determine the best way to assess the effects of light on cellular nitrite-dependent nitric oxide production. These included: investigating different types of light source, controlling for temperature, varying the time of addition of substrate (nitrite), and examining the time and duration of illumination. After preliminary studies with these variables a 50 watt Xenon/Halogen flood light (Feit Electric Co.) capable of producing broad spectrum visible and near IR light was used. Spectral emission from this bulb (FIG. 2) was determined using an Ocean Optics Diode Array Fiber Optic spectrophotometer (Model SD 2000) by personnel in the Integrated Instrument Development Facility of CIRES lab at the University of Colorado, Boulder.

Cells being assayed were kept at a constant temperature of 28° C. in a water jacketed chamber and a heat filter was placed between the light source and the cells in order to insure that the effects observed were due to light and not a change in temperature due to illumination. Light intensity at the surface of the assay chamber was measured with a Newport Instruments 918D-SL Power meter. All studies were done in a darkened room. Cells were exposed to a light intensity of 7 mW/cm², which corresponds to setting the light bulb 20 inches from the assay chamber. The length of time cells were exposed to light was varied in order to deliver variable levels of total light energy. Prior to exposure to light the cell suspension was sparged with nitrogen gas to remove oxygen. They were then exposed to light for variable times and then nitrite was added to start the reaction. Nitric oxide levels were measured with a nitric oxide electrode attached to a WPI Apollo 4000 nitric oxide meter.

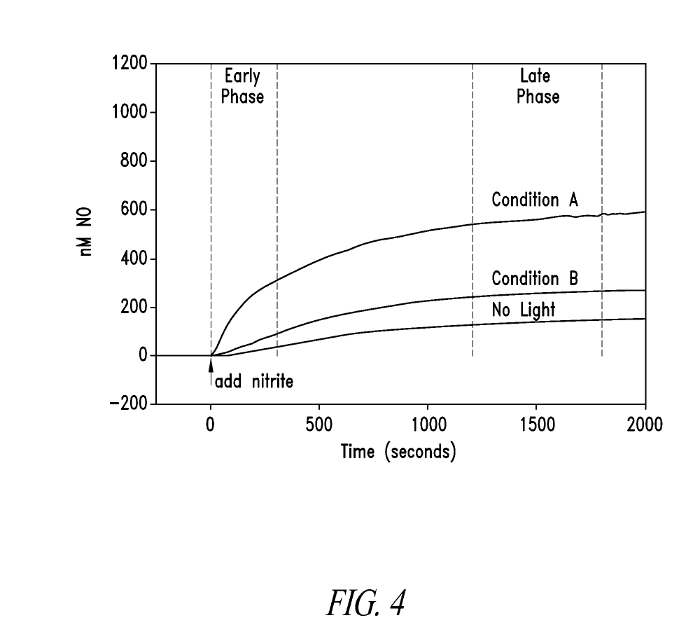

As shown in FIG. 3, two experimental conditions were tested. In Condition A cells were pre-conditioned by exposure to light for variable lengths of time, prior to the addition of nitrite. Upon addition of nitrite the light was turned off. Condition B was the same as Condition A except that the light was kept on for the duration of the experiment. The effect of broadband light on nitrite-dependent nitric oxide production under Conditions A and B is shown in FIG. 4. By comparing nitric oxide production under Conditions A and B with nitric oxide production in the absence of light it is clear that broadband light stimulates nitrite-dependent nitric oxide production in hypoxic cells under both Conditions A and B and that there are two distinct phases. The initial phase is characterized by the rapid production of nitric oxide. This phase is followed by a slower phase. For convenience, the initial phase is termed the “early phase” and the second phase is termed the “late phase”. It is not known why the rate slows but is likely that the overall level of nitric oxide produced is determined largely by the enhanced rates observed during the early phase. Although either phase can be used for these studies, the late phase rates and overall levels of nitric oxide production are more reproducible than the early phase rates. From FIG. 4 it is obvious that the additional light energy received during Condition B gives less enhancement on the rate of nitric oxide production than the protocol followed in Condition A. Indeed, the pre-conditioning step with light in Condition A seems to be sufficient. Because of this, all subsequent studies have been done using Condition A.

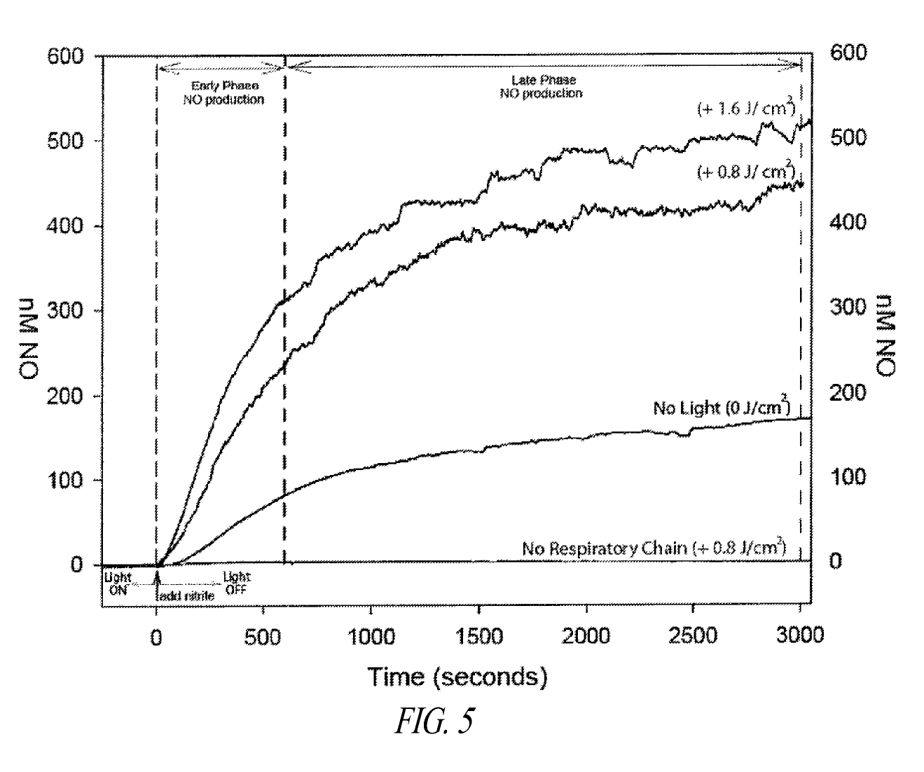

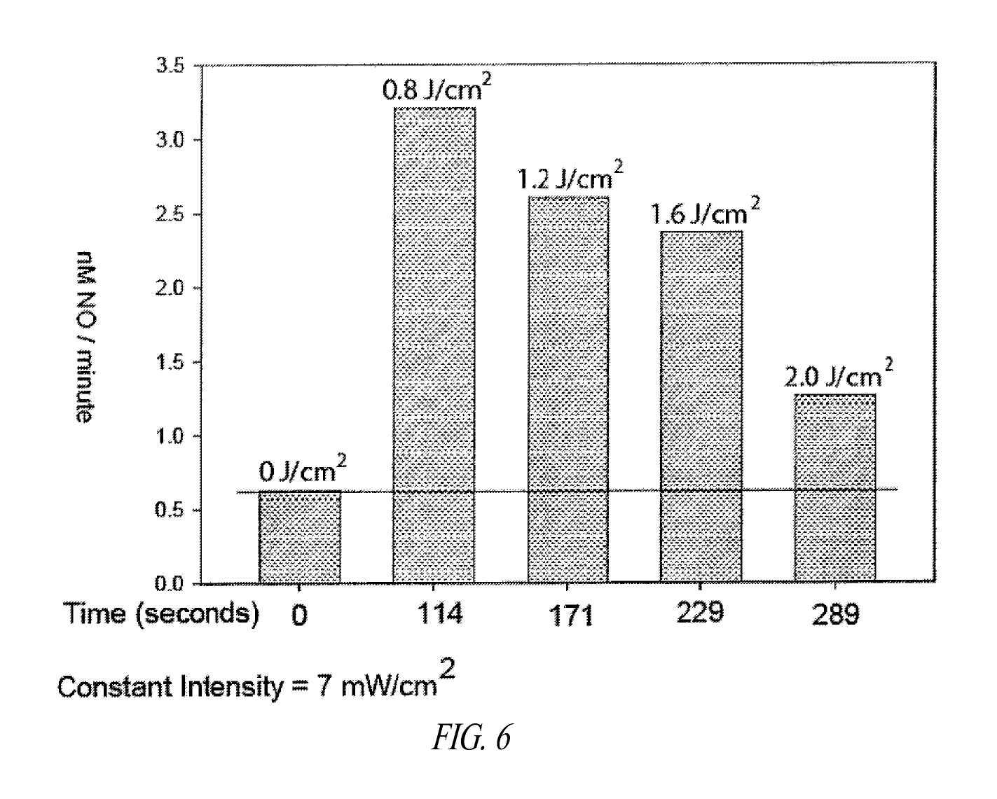

The effect of light intensity on nitrite-dependent nitric oxide production by yeast cells was determined by varying the exposure time during the pre-conditioning phase. From FIG. 5, it is clear that the stimulatory effect of light on nitrite-dependent nitric oxide production requires the respiratory chain because it is not observed in a strain that is respiration-deficient. It is also clear that increasing light intensity from 0.8 to 1.6 J/cm2 increased the early phase rate of nitric oxide production. A more complete analysis of the effects of light intensity on the rates of nitrite-dependent nitric oxide production during the late phase is shown in FIG. 6. Maximum stimulation of the rates of nitric oxide synthesis are observed at light intensities of 0.8 J/cm2. A similar relationship between light intensity and nitric oxide production similar was observed for nitric oxide synthesis during the early phase.

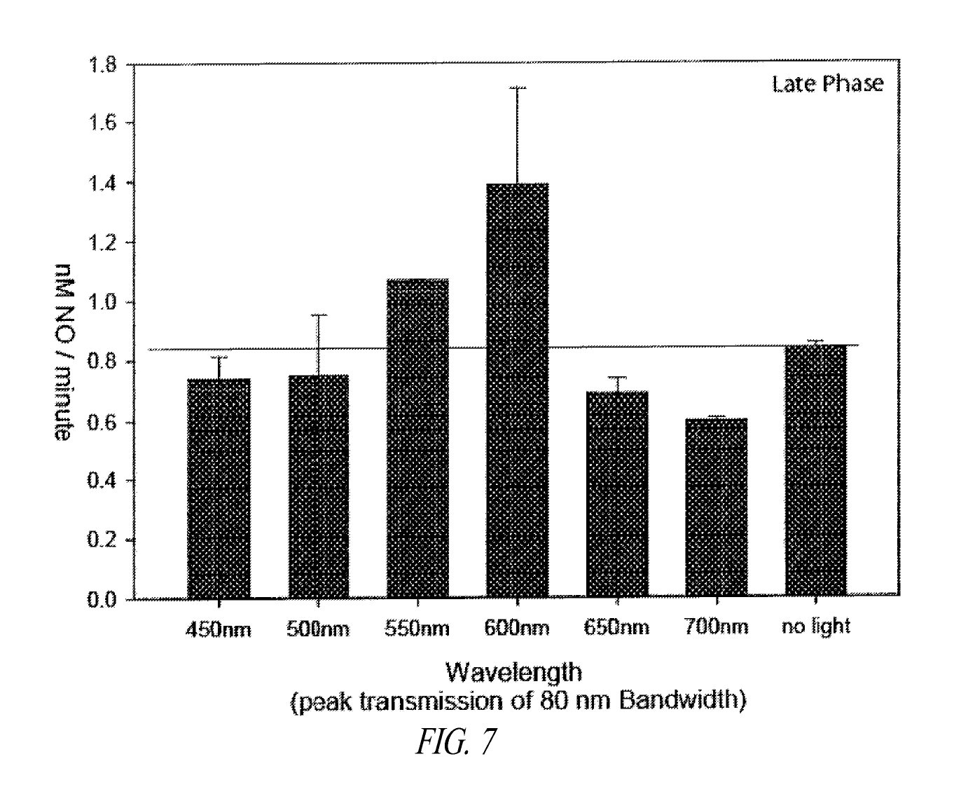

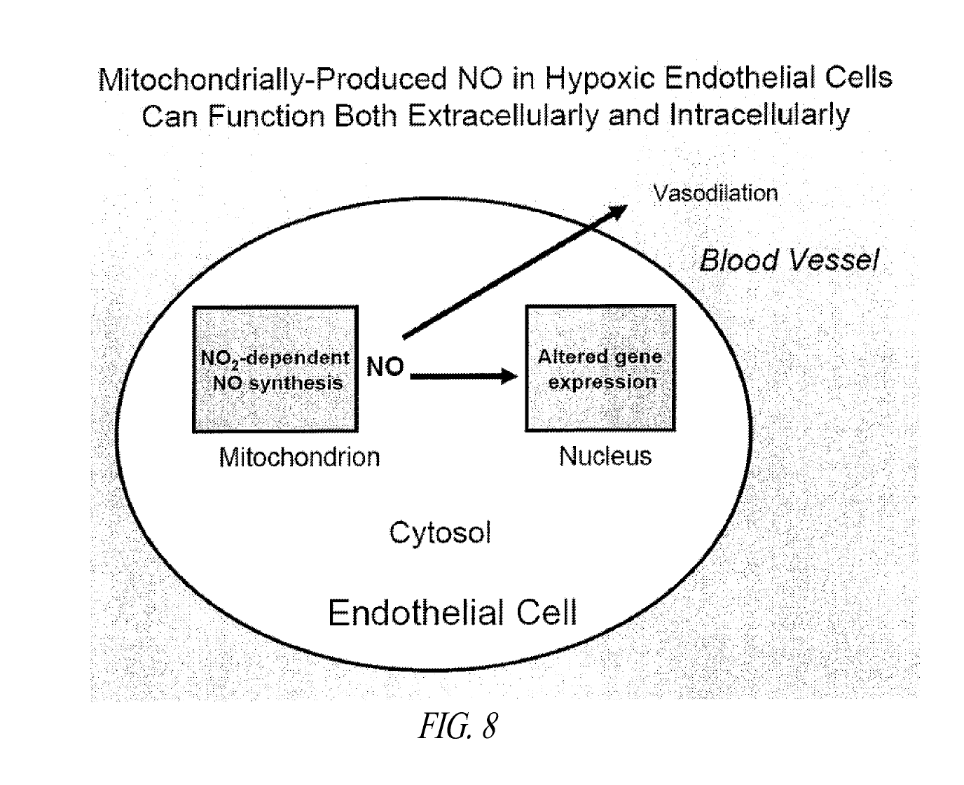

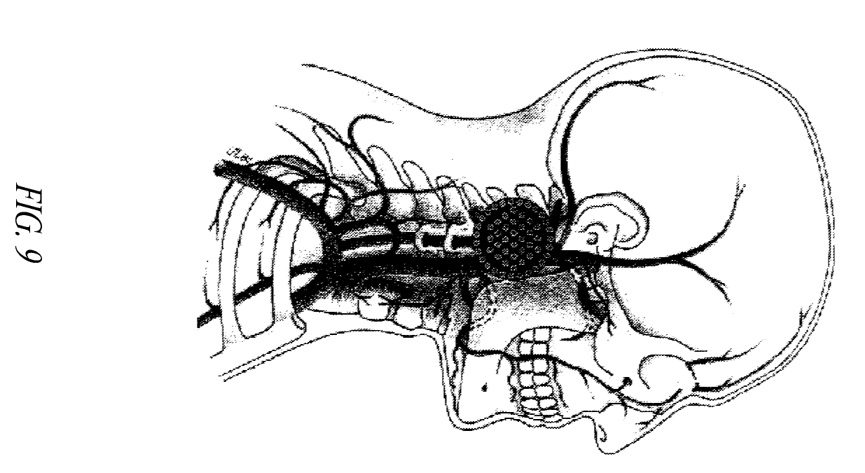

A series of broadband interference filters from Edmund Scientific were used to assess the effects of specific wavelengths of light on nitrite-dependent nitric oxide production and hence produce the used action spectrum. These filters had peak transmittance every 50 nm and a full width half maximum bandwidth (FWHM) of 80 nm. The overall rates of nitric oxide production during the late phase are shown in FIG. 7. Maximum stimulation of nitric oxide production was observed when cells were stimulated with the 550±40 nm and 600±40 nm filters. Wavelengths transmitted by the 450 and 500 nm filters had no effect on nitric oxide production. Surprisingly, those wavelengths transmitted by the 650 and 700 nm filters light had an inhibitory effect on nitrite-dependent nitric oxide production when compared to the no light control. In order to further refine the wavelength dependence of both the stimulatory and inhibitory effects of light on nitrite dependent nitric oxide production we used narrow bandwidth interference filters from Cheshire optical. These filters had center wavelengths spaced every 10±2 nm and covered the range between 530 and 850 nm. Unfortunately, because these narrow band filters reduce the level of light transmission to a level that is below that required for light stimulated nitric oxide synthesis they were not suitable for establishing a higher resolution action spectrum.

The results obtained from the above studies clearly support the conclusion that broadband light affects nitrite-dependent nitric oxide production in yeast cells and does so in a dose-dependent fashion. They also support the conclusion that some wavelengths of light are stimulatory while others are inhibitory. In addition, the experiments performed during the past 3.5 months have indicated that while light bulbs can be used for these studies they suffer from the disadvantage that their output spectra change as they age. This is inconvenient and suggests that alternative sources of light energy (e.g., LEDs) will be more appropriate for future studies.

### Example 9

### NO Production

Production of NO via the Cco pathway in human endothelial cells and murine brain tissue. Human microvasculature endothelial cells (HMVEC) and human umbilical endothelial cells (HUMVEC) and mitochondria from murine brain tissue mitochondria are all found to be capable of nitrite-dependent NO synthesis catalyzed by Cco. A representative experiment with brain mitochondria is shown in FIG. 12. This reaction requires nitrite, does not occur at oxygen concentrations above 10 μM 0₂, and the NO produced is removed by PTIO, an NO scavenger. As found for yeast, this reaction is stimulated by light in an intensity dependent fashion. It is also differentially affected by different wavelengths of light tuned to specific absorption bands of Cco.

These findings establish, for the first time, that mammalian endothelial and murine brain mitochondrial Cco possess the ability to catalyze nitrite-dependent NO synthesis under hypoxic conditions. Accordingly, this pathway is available for NO synthesis under the hypoxic conditions that accompany a variety of patho-physiological conditions in brain, including those that precede some sporadic types of AD. They also demonstrate that this pathway for NO synthesis can be dramatically affected by light.

### Example 10

### Alzheimer's Disease Treatment and OLED Device

Alzheimer's disease (AD) is an increasingly prevalent form of senile dementia known for the memory loss, cognitive failures and behavioral changes in patients with the disease. One of the most common physiological changes in the brains of AD patients is the appearance of extra cellular fibrillar plaques near regions of neurodegeneration. The extracellular plaques are composed mostly of insoluble, aggregates of a short peptide fragment, identified as the Amyloid Beta (Aβ) peptide. This proteolytic fragment of the much larger Amyloid Precursor Protein (APP) occurs most abundantly in 40 and 42 amino acid residue lengths. The connection between Aβ and AD has been strengthened by the fact that many of the mutations associated with FAD promote the production of Aβ from APP (Selkoe, 2001). For example, mutations in the Presinilin genes, which are involved in proteolysis of APP leading to Aβ fragments, are linked to FAD by family pedigrees (Sherrington et al., 1996). While there is clear evidence for the involvement of APP and Aβ fragments in AD, the exact molecular mechanism of there action remains elusive. Importantly, there has been wide recognition of the link between oxidation stress and the progression of Alzheimer's.

Many disorders of the cardiovascular system result in persistent hypoxia and even anoxia, as a consequence of stroke. These conditions are capable or reducing or completely abolishing oxygen levels in the brain (in the case of stroke) for short periods of time. It is now well-known that patients suffering from these cardiovascular disorders have increased susceptibility to Alzheimers disease. Indeed, reduced cerebral perfusion is an AD risk factor. This suggests that endothelial dysfunction and vasoconstriction contribute to the development and progression of AD. The hypoxia that results from vasoconstriction could easily account for the enhanced oxidative and nitrosative stress that accompanies AD (Beal, 2000; Perry et al., 2002; Polidori et al., 2007). Indeed, it is now well established that hypoxia induces the transient generation of reactive oxygen species (ROS), especially superoxide, by the mitochondrial respiratory chain (Chandel et al., 2000; Dirmeier et al., 2002; Grishko et al., 2001). These ROS are generated is large quantity in hypoxic cells and some of them are released into the blood where one of them, superoxide, can react with blood NO to produce peroxynitrite. This series of events has at least three consequences: 1) the increased levels of ROS lead to enhanced levels of oxidants and oxidative stress, 2) the peroxynitrite that is generated can tyrosine nitrate proteins, which can modify their function result in increase nitrosative stress, and 3) there is a decrease in the effective concentration of NO available for blood vessel dilation This reduction in NO levels results in vasoconstriction and limited blood flow to the brain. Each of these has been observed in AD. Given the role of hypoxia in AD progression it is clear that any therapy that can enhance NO levels under hypoxic conditions could relax vasoconstriction, improve oxygen deliver to the brain, and limit brain nitrosative and oxidative stress. This, in turn, would slow or reverse the progression of AD.

Nitrite-dependent NO synthesis catalyzed by cytochrome c oxidase (Cco). Cco is the terminal protein of the mitochondrial respiratory chain in all mammalian cells. Until recently, mitochondrial Cco was thought to have only one enzymatic activity; the reduction of oxygen to water. This is an oxidase reaction that occurs under normoxic conditions and involves the addition of 4 electrons and 4 protons to diatomic oxygen. Recently, we discovered a second enzymatic function for eukaryotic Cco (Castello et al., 2006; Castello et al., 2008). This activity involves the reduction of nitrite to nitric oxide (NO). This nitrite reductase activity of Cco is favored under hypoxic conditions, is inhibited by oxygen, and is enhanced by low intracellular pH. So far, this reaction has been demonstrated in yeast cells and yeast mitochondria, rat liver mitochondria, mouse neuronal cells and mitochondria, human endothelial cells and mitochondria, and the mitochondria of hypoxic plant roots. Its presence in a wide variety of organisms and cells suggest that it is a universal method for NO synthesis under hypoxic conditions, like those that accompany several pathophysiological states.

The overall goal of this Example is to illustrate how to evaluate the efficacy of various photo-biomodulation therapies for the treatment of Alzheimer's disease.

As disclosed above nitrite-dependent nitric oxide (NO) synthesis catalyzed by cytochrome c oxidase (Cco) is a primary source of nitric oxide synthesis under the hypoxic/anoxic conditions that occur during a variety of neurological diseases. The importance of this mechanism of NO synthesis during hypoxia has been demonstrated in a wide range of cell types including yeast, (rat) liver, (human) endothelial cells and (mouse) neurons. NO synthesis by Cco can be activated by light tuned to specific absorption bands of Cco. In accordance with the strong epidemiological connection between oxidative stress and Alzheimer's disease (Zhu and Chiappinelli, 1999), this experiment is to illustrate the effectiveness of activating the Cco/NO pathway in the treatment of Alzheimer's disease by use of an OLED patch-based treatment device and in two established Alzheimer's mouse models.

Patch OLEDs designed for mouse carotid or brain irradiation with optimal emission wavelengths of 550, 600, and 650 nm are used. These devices are powered by single 1600 mA/hr batteries that will achieve operational lifetimes of weeks to months.

Several established mouse model's of familial Alzheimer's disease including the 5×FAD, and the Swedish mutation model crossed with a NOS2−/− strain are used in testing the effectiveness of the photobiomodulation devices fabricated in Aim I to delay cognitive decline over a 5 month period. A broad range of treatment regimes are explored and mice are assayed using both the Y-maze and Morris water maze test for cognitive decline. Once an optimal treatment regime is established based upon cognitive tests the optimal treatment regime are repeated in both AD and control mice over another 5 month period and subjects are reassessed for cognitive decline and a small subset are to be removed for biochemical and pathological analysis.

Using the brain tissue from the mice above, β-amyloid Elisa assays are performed to determine changes in amyloid peptide production following treatment. APP pulldown and mass-spec analysis are used to determine changes in APP processing. The oxidase and nitrite reductase activites of Cco activities in brain tissue taken from light-treated and untreated mice are determined. Measurements of serum NO levels in light-treated and untreated mice are also made. Finally, histological analysis of brain tissue monitor the progression of the disease and the accumulation of amyloid plaques.

The first mouse model (Oakley et al., 2006) is the 5×FAD transgenic mouse that overexpresses both mutant human APP (695) with the Swedish (K670N, M671L), Florida (1716V), and London (V7171) Familial Alzheimer's Disease (FAD) mutations and human PS I harboring two FAD mutations, M 146L and L286V. These mice accumulate intraneuronal Abeta-42 starting at 1.5 months of age, just prior to amyloid deposition and gliosis, which begins at 2 months of age. In addition, these mice have reduced synaptic marker protein levels, increased p25 levels, neuron loss, and memory impairment in the Y-maze test and rapidly recapitulate major features of Alzheimer's disease amyloid pathology. The second model (Colton et al., 2006) to be used is a cross between the APPSw expressing transgenic mouse and the NOS2−/− mouse which results in enhanced disease progression and provides a key link between appropriate NO levels and Alzheimer's. This model allows one to establish the effectiveness of particular treatment methods and regimes at restoring appropriate NO levels in the brain.

To determine the functional significance of this progression, three behaviors that are known to depend on the integrity of the hippocampus: (a) Spatial alternation, (b) Exploration, and (c) Place Learning are used. Accordingly, the treatment protocol is a matrix that provides for a broad initial screen of treatment regimes that affect disease progression. Following this initial screen a large scale more in depth study is to be performed coupled to the biochemical and pathological studies outlined below.

Treatment protocols which adjust both irradiation wavelength and dosage matrix are employed (see, table below). Animals are shaved and the OLED adhered using the bio compliant glue 2-octyl cyanoacrylate. The OLED are monitored for 1 day and then activated by insertion of the battery to begin the treatment regime. Each individual OLED is numerically coded during the fabrication based upon both the emission wavelength and the irradiation protocol. This allows for efficient bookkeeping during cognitive tests.

| Wavelength | Dosage Regime (Joules/cm²/Day) | | | | | | | | | |
|---|---|---|---|---|---|---|---|---|---|---|
| 550 nm | 5 | 10 | 15 | 20 | 25 | 30 | 35 | 40 | 45 | 50 |
| 600 nm | 5 | 10 | 15 | 20 | 25 | 30 | 35 | 40 | 45 | 50 |
| 650 nm | 5 | 10 | 15 | 20 | 25 | 30 | 35 | 40 | 45 | 50 |

Cognitive testing using the Y-Maze. Each mouse is tested once a day for 14 days. The test starts by placing the mouse in one of the 3 arms of the Y maze and allowing it to enter of one of the two other arms (say arm a) where it is trapped for 30 sec. The mouse is then returned to the start arm where it is released and allowed to make a choice to enter one of the two available arms. If it chooses to enter a different arm (say arm b) on the second component test outcome is scored as alternation trial. Normal mice will alternate on about 80% of the 14 test trials. Mice from each of the three strains is tested at 3 ages (1 month, 2 months and 4 months).

Cognitive Testing using the Morris Water Maze. The water task consists of a circular galvanized steel pool approximately 117 cm in diameter and 58 cm deep. A movable escape platform constructed of a Plexiglas base column, having a height of 43 cm and topped by a round platform 15 cm in diameter, is placed in one quadrant of the pool and will be maintained there throughout acquisition of the task. The mouse is trained to find the hidden platform. Training consists of 4 trials a day. On a trial, the mouse is taken from its cage and place into the pool for 40 seconds or until it finds the escape platform. If it does not find the platform in 40 sec, it will be guided to the platform by the experimenter. 15 sec after reaching the platform the mouse will be gently dried and returned to its cage. The interval between trials is approximately 15 min. Another cohort of mice from each strain will also be tested at 3 different ages on this task (1, 2, and 4 months). Determining the levels of beta-amyloid peptides. Brain tissue derived from treated and untreated mice is to be analyzed for levels of two primary amyloid peptides (Aβ1-40 and Aβ1-42) using Elisa methods. Tissue samples will be homogenized and extracted and total protein concentration determined. Elisa assays will be performed using the Invitrogen protocol to determine amyloid peptides Aβ1-40 and Aβ1-42 concentration and normalized to total protein concentration in the extracts.

Determining changes in APP processing. Brain tissue derived from treated and untreated mice is to be analyzed using MS/MS of APP immunoprecipitates. The anti-c-terminal APP antibody will be used to immunoprecipitate APP from tissue lysate. The immunoprecipiate is then used for MS/MS analysis to determine the types and extent of APP processing occurring. The quantitative MS/MS analysis allows the types and degrees of APP processing in treated and untreated mice to be monitored.

Histochemical analysis of brain slices. Using previously described methods (Oakley et al., 2006), the pathological progression of treated and untreated mice is analyzed to monitor the extent and location of amyloid deposition, glioses, and neuronal loss.

Measurement of Cco/NO activity. For these studies brain tissue from the cerebrum of light-treated and untreated mice is used. Mitochondria are isolated and assayed for nitrite-dependent NO synthesis under hypoxic conditions. To assure that NO synthesis from Cco is being measured electron donor pair (ascorbate/TMPD) with a redox potential that allow electrons to be fed to cytochrome c and then Cco is used. This obviates the need for the earlier electron carriers in the respiratory chain. This activity is measured in the presence and absence of light from a broadband xenon/halogen bulb.

Determination of serum NO levels in light-treated and untreated mice. To determine if light exposure has systemic effect on NO levels in mice, blood is taken from the tail vein of mice weekly and NO levels determined colorimetrically using the Greiss reagent. This allows quantitatively monitoring of the affects on photobiomodulation upon NO levels.

Thereby the effects of various OLED photobiomodulation methods on Alzheimers and various biomarkers important for Alzheimer's can be determined.

### Example 11

### Cco of Endothelial and Nerve Cells Response to Light Under Hypoxic Conditions, Including an Alzheimer's Disease Model

The current example investigated the ability of mitochondrial Cco of endothelial cells and nerve cells to make NO from nitrite under hypoxic conditions. In addition, the study investigated the effects of light on mitochondrial Cco/NO activity in both human endothelial cell and mouse cerebrum mitochondria. Finally, the study investigated the alteration of Cco/oxidase or Cco/NO activities in the ‘Swedish’ mouse model for Alzheimer's disease and examined the ability of light to enhance Cco/NO activity in this model.

The principal aims of this example were:

1) Evaluation of exposure to light on the expression of the nuclear genes for yeast cytochrome c oxidase and the overall level of yeast cell respiration.

2) Determination of endothelial and nerve cell mitochondria capabilities for nitrite-dependent NO synthesis.

3) Examination of the effects of light on mitochondrial NO synthesis in endothelial and nerve cells.

4) Establishing that mitochondrial NO synthesis is altered in a mouse model carrying the ‘Swedish’ mutation in the amyloid protein for Alzheminer's disease and determining that light stimulates mitochondrial NO synthesis in the model.

### Aim 1: Effects of Exposure to Light on COX Gene Expression and Respiration.

The effects of broad spectrum light on the expression of the nuclear COX genes for Cco and the overall level of cellular respiration were examined. Because cytochrome c oxidase is a major regulator of respiration and because the nuclear genes (COX) for cytochrome c oxidase are highly regulated by a variety of environmental factors and function to regulate the overall activity of Cco, their level of expression provides a useful measure for the long-term effects of light on Cco. Similarly, the rate of respiration per se provides a direct measure of the effects of light on the entire respiratory chain. After an initial period of testing different experimental protocols we settled on examining the effects of broadband light on COX gene expression and respiration in yeast cells incubated in 4 different conditions: (1) a control in which cells were not treated with either nitrite or light, (2) a nitrite sample in which cells were treated with 1 mM nitrite, (3) a light-treated sample where cells received three separate doses of light (each of 6.4 J/cm²), and (4) a final sample which received the combination of nitrite and light (same amount and dosages as in (3)).

Yeast cells were grown in liquid media for three hours, which is equivalent to the time it takes for one cell division. Some cells were grown in the presence of oxygen (normoxia) while others were grown at low oxygen levels (hypoxia) in each of the four conditions described above. For hypoxic cultures, a constant gas flow of 1% O₂, 5% CO₂, 94% N₂ was administered beginning one hour before the experiment began. Gas flow remained constant during the experiments. Nitrite-supplemented samples were dosed 1 hour before the beginning of each experiment.

Light from a Broadband 50 watt Xenon/Halogen Floodlight was administered at the beginning of each hour for a total of 3 doses, each at 6.4 J/cm² (105 mW/cm²). After 3 hours, cells were harvested and analyzed for measurement of gene expression (by quantitiative PCR), or respiration (by polarographic oxygen consumption). qPCR was performed with primers designed for COX of Cco subunits COX4, COX5a, COX5b, COX6, and COX8. Each sample was run in duplicate and gene expression was quantified by normalizing to PMAI, a yeast gene that is expressed at constant levels, irrespective of the environment in which cells are grown. The gene expression data are presented in Table 1.

TABLE I
Effects of Exposure to Light and Nitrite
on Nuclear COX gene expression

| | Normoxia | | Hypoxia | |
|---|---|---|---|---|
| | | Fold | | Fold |
| | Relative | Change | Relative | Change |
| Sample | Expression** | (%) | Expression** | (%) |
| Control | 1.00 | — | 1.00 | — |
| +Nitrite | 1.024 ± 0.067 | n.c.* | 0.808 ± 0.031 | −19.2 |
| +Light | 1.004 ± 0.080 | n.c. | 0.679 ± 0.037 | −32.1 |
| +Light and | 0.983 ± 0.043 | n.c.. | 1.334 ± 0.048 | +13.4 |
| Nitrite | | | | |

*N.C. = no change
**Average values for combined expression of COX4, COX5a, COX6, and COX8

Under normoxic conditions neither light, nitrite, nor a combination of light and nitrite produced a significant change in COX gene expression. However, under hypoxic conditions, light and nitrite were inhibitory, while the combination of both light and nitrite had a stimulatory effect. These results are surprising because COX4, COX5a, COX6, and COX8 are aerobic genes whose expression is maximal under normoxia. They suggest that light and nitrite can override oxygen as regulator of these genes. From Table 2 it can be seen that light has little effect on whole cell respiration levels under normoxia but that the combination of light and nitrite promotes a higher level of respiration in cells incubated under hypoxia. The addition of nitrite by itself has a slight effect. Interestingly, the 13.4% increase in the level of respiration in cells exposed to light and nitrite under hypoxia parallels the increase observed in the expression of the nuclear COX genes.

TABLE 2
Effects of exposure to nitrite and light on yeast cell respiration

| | Normoxia | | Hypoxia | |
|---|---|---|---|---|
| | Oxygen Con- | Fold | Oxygen Con- | Fold |
| | sumption (nmol/ | Change | sumption (nmol/ | Change |
| Sample | min/mg cells) | (%) | min/mg cells) | (%) |
| Control | 14.77 ± 0.50 | — | 13.76 ± 0.15 | — |
| +Nitrite | 15.21 ± 0.47 | n.c..* | 14.27 ± 0.26 | +3.7 |
| +Light | 14.95 ± 0.34 | n.c. | 33.71 ± 0.33 | n.c. |
| +Light and | 13.85 ± 0.24 | −6.3 | 15.61 ± 0.31 | +13.4 |
| Nitrite | | | | |

* = no change

The above findings indicate that intermittent exposure of yeast cells to broadband light can affect the expression of some genes as well as the overall level of respiration. This light effect is observed only under hypoxic conditions. It is possible that a more prolonged exposure to light would yield even greater effects on gene expression and/or respiration in hypoxic cells. It is also possible that effects of light would be even greater after a longer duration of growth post exposure.

### Aim 2: Cco/NO Activity in Endothelial Cells and Nerve Cells.

We have studied Cco/NO activity in both human endothelial cells and mouse brain nervous tissue. All of the assays for Cco/NO activity were done in a closed chamber, as described elsewhere (Castello et al. 2006, Cell Metabolism 3, 277-287). Cells or mitochondria were allowed to respire and consume oxygen all of the oxygen in the chamber before the addition of nitrite, which initiated the reaction. It is important to note that NO synthesis begins only after the chamber becomes hypoxic (i.e., the oxygen concentration in the chamber was reduced significantly—less than 10 μM O₂).

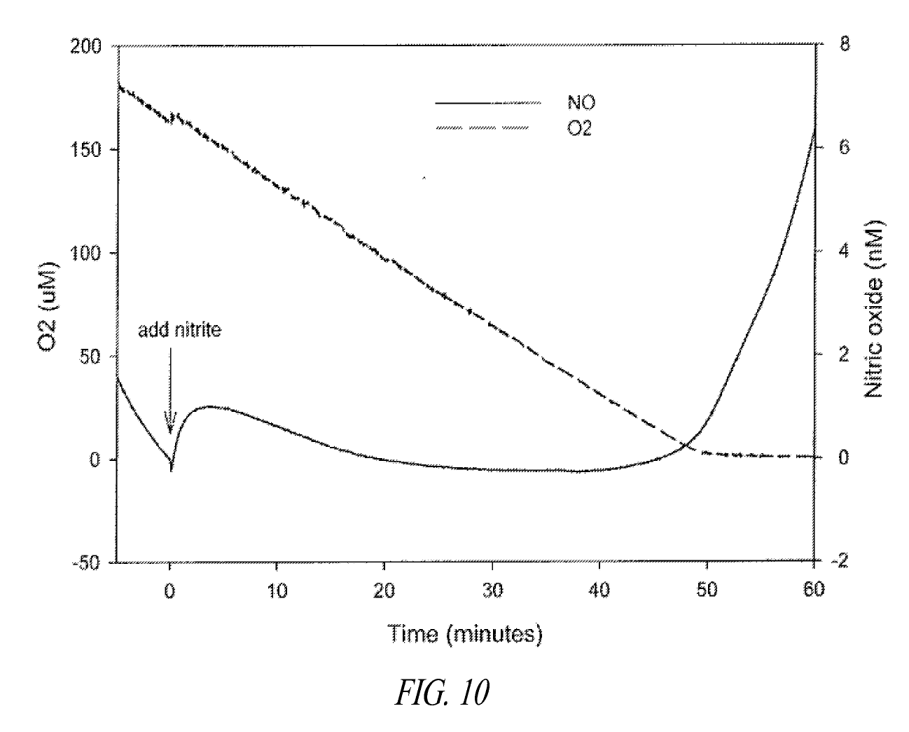

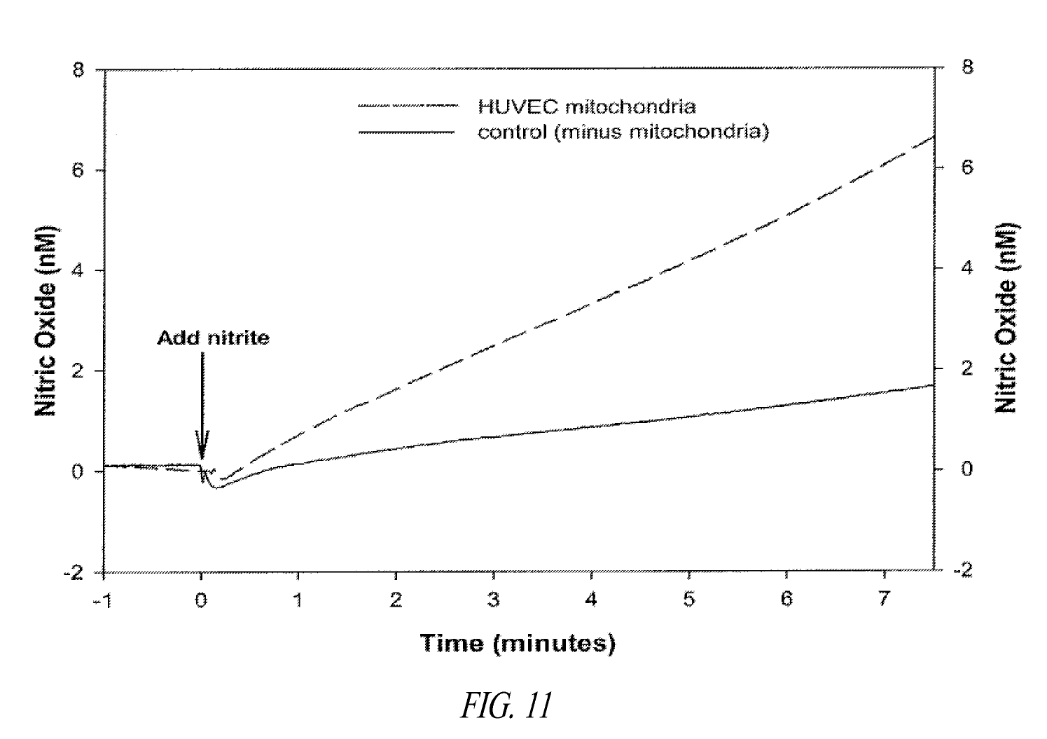

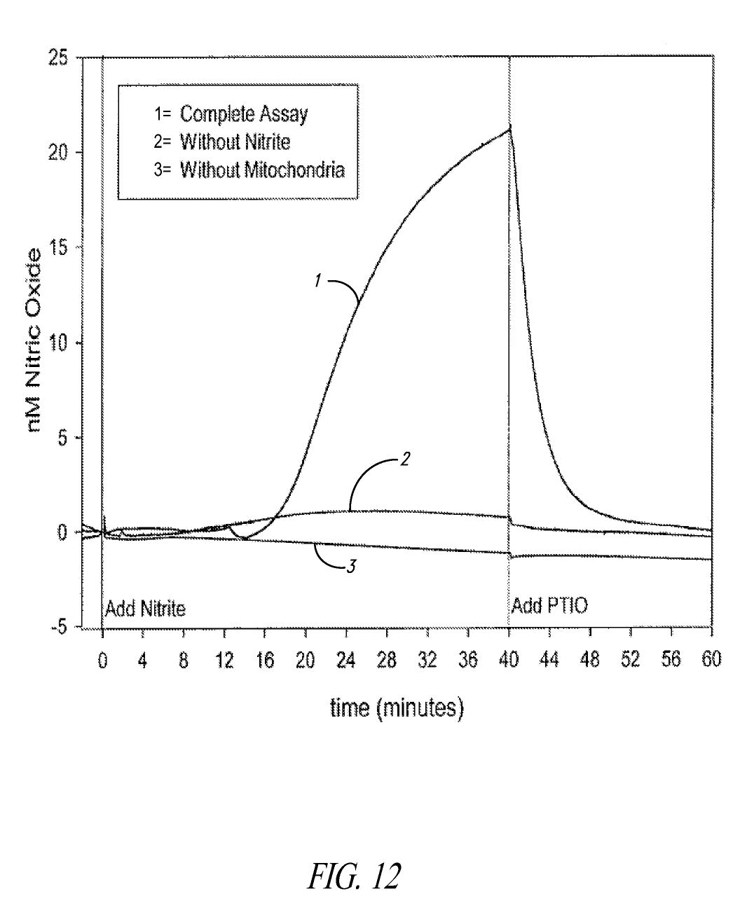

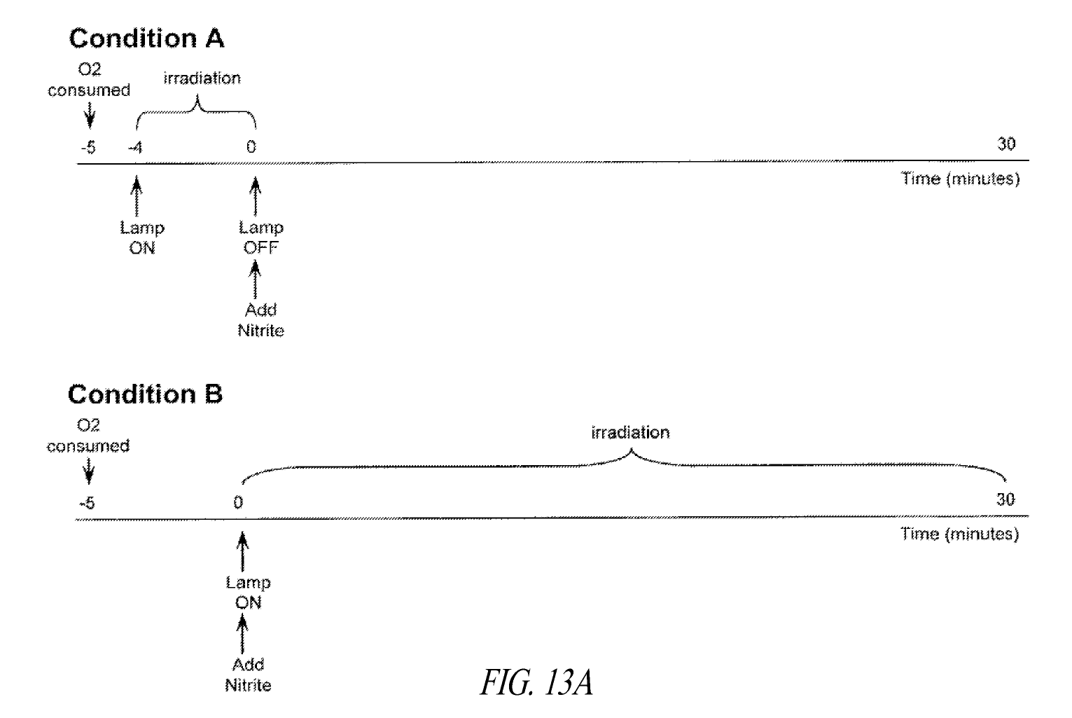

Results with human endothelial cells. For these studies, we used both human microvascular endothelial cells (HMVEC) and human umbilical vein endothelial cells (HUVEC). To measure nitrite-dependent NO synthesis, cells were grown in tissue culture plates to confluence, harvested, and placed in a closed chamber where they were allowed to consume the available oxygen in the chamber by respiration. Nitrite was then added and NO production measured with a sensitive NO electrode. A representative experiment with HMVEC cells is shown in FIG. 10. It can be seen that NO synthesis does not begin until the oxygen concentration in the chamber drops significantly. Similar results were obtained with HMVEC cells. To determine if this reaction was catalyzed by mitochondrial Cco oxidase we first isolated mitochondria from HUVEC cells and then measured Cco/NO activity, using an assay with the electron donor pair ascorbate/TMPD that feeds electrons into the terminal portion of the mitochondrial respiratory chain (e.g., Castello et al. 2006, Cell Metabolism 3, 277-287). The data in FIG. 11 demonstrate that HUVEC mitochondrial Cco possesses Cco/NO activity.

Results with mouse nerve cells. These experiments used mitochondria isolated from the cerebrum of 7-12 day old mice. Using the same assay we used for endothelial cell mitochondria, we assayed Cco/NO activity under hypoxic conditions with ascorbate/TMPD as an electron donor pair. From FIG. 12, it is clear that, like the endothelial cell mitochondria discussed above, neuronal mitochondrial Cco is capable of NO synthesis and that this reaction requires nitrite. To confirm that we had measured NO synthesis we added PTIO, an NO scavenger. As can be seen in FIG. 12, TIO completely removes the NO produced by mitochondrial Cco.

The findings from Aim 2 establish, for the first time, that mammalian endothelial and nerve cell mitochondrial Cco possesses the ability to catalyze nitrite-dependent NO synthesis under hypoxic conditions. This result indicates that this pathway is available for NO synthesis under the hypoxic conditions that accompany a variety of patho-physiological conditions. The results also allow us to extend the list of available species that possess this pathway to include not only yeasts and plants, but mammals. This list now includes yeast, Dictyostelium (a cellular slime mold), rice, wheat, mice and humans.

### Aim 3: Effects of Light on Mitochondrial Cco/NO Activity in Endothelial and Nerve Cells.

These experiments investigated light stimulation of Cco/No activity in endothelial and nerve cells.

Endothelial Cells.

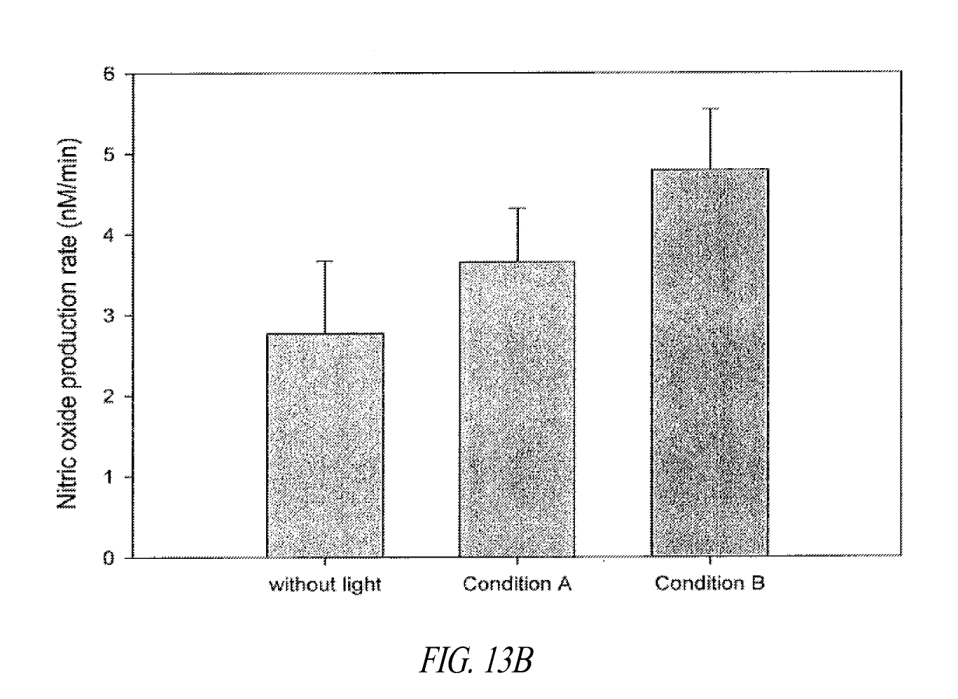

To demonstrate the effect of investigate the ask if light has an effect on nitrite-dependent NO synthesis in mammalian cells intact HUVEC cells were used. The source of illumination used was a 50 watt Xenon/Halogen Floodlight. This light source is capable of producing broad spectrum visible and near IR light. Two experimental conditions were examined to assess the effects of light on nitrite-dependent NO synthesis in hypoxic cells. In Condition A, cells were pre-conditioned by exposure to light for variable lengths of time, prior to the addition of nitrite (FIG. 13A). Upon addition of nitrite, the light was turned off. Condition B was the same as Condition A except that the light was turned on at the same time that nitrite was added and kept on for the duration of the experiment. For both conditions, the cells used were from confluent cultures and they were incubated in the assay chamber until the chamber became hypoxic, at which point they were exposed to light. Cells being assayed were kept at a constant temperature of 37° C. in a water jacketed chamber and a heat filter was placed between the light source and the cells in order to insure that the effects observed were due to light and not a change in temperature due to illumination. Light intensity at the surface of the assay chamber was measured with a Newport Instruments 918D-SL Power meter. All studies were done in a darkened room. Cells were exposed to a light intensity of 4 mW/cm², which corresponds to setting the light bulb 20 inches from the assay chamber. The effect of broadband light on the nitrite-dependent NO synthesis under Conditions A and B is shown in FIG. 13B. Both light regimes support higher rates for nitrite-dependent NO synthesis. However, the difference between the results from Condition A or B and the control without light does not appear to be statistically significant. Therefore, studies aimed at determining the effects of different light intensities and wavelengths on this reaction in endothelial cells were not pursued.

Nerve Cells.

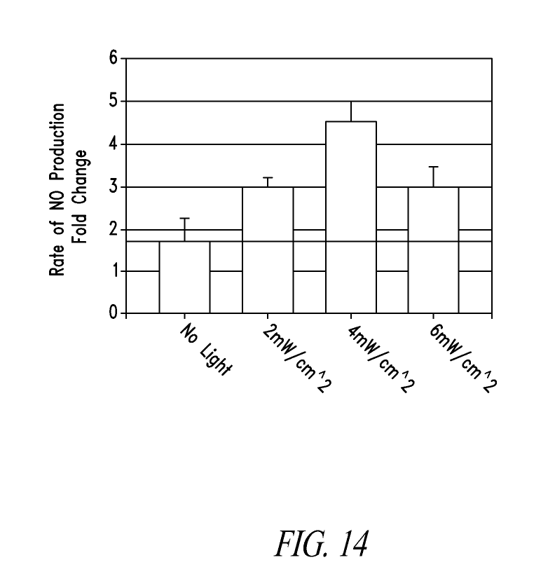

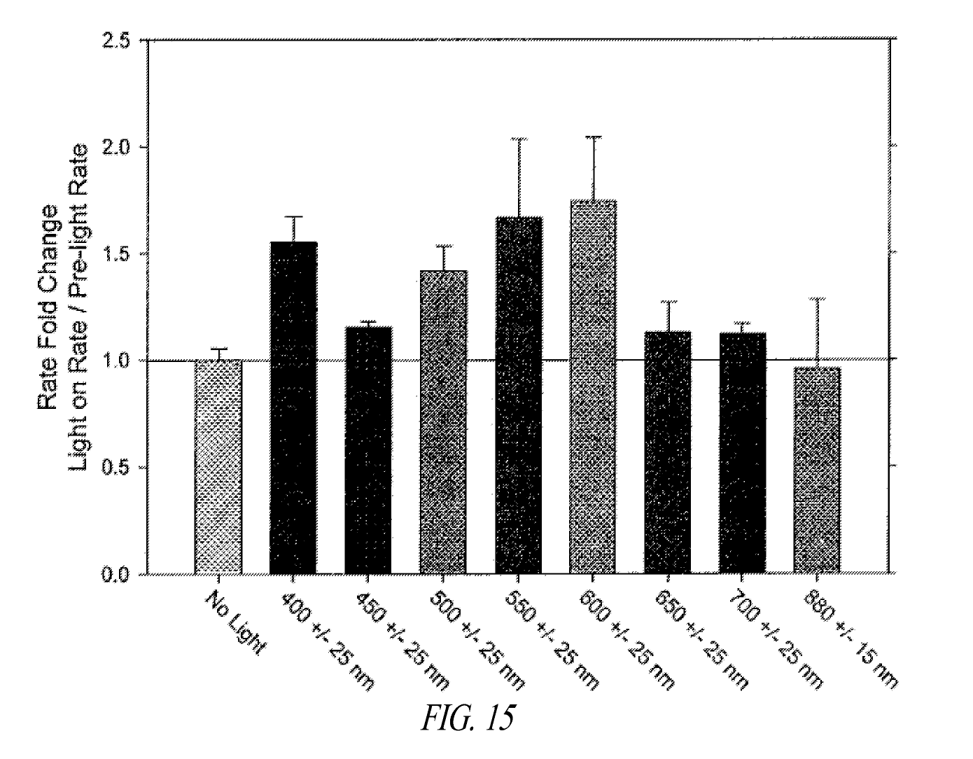

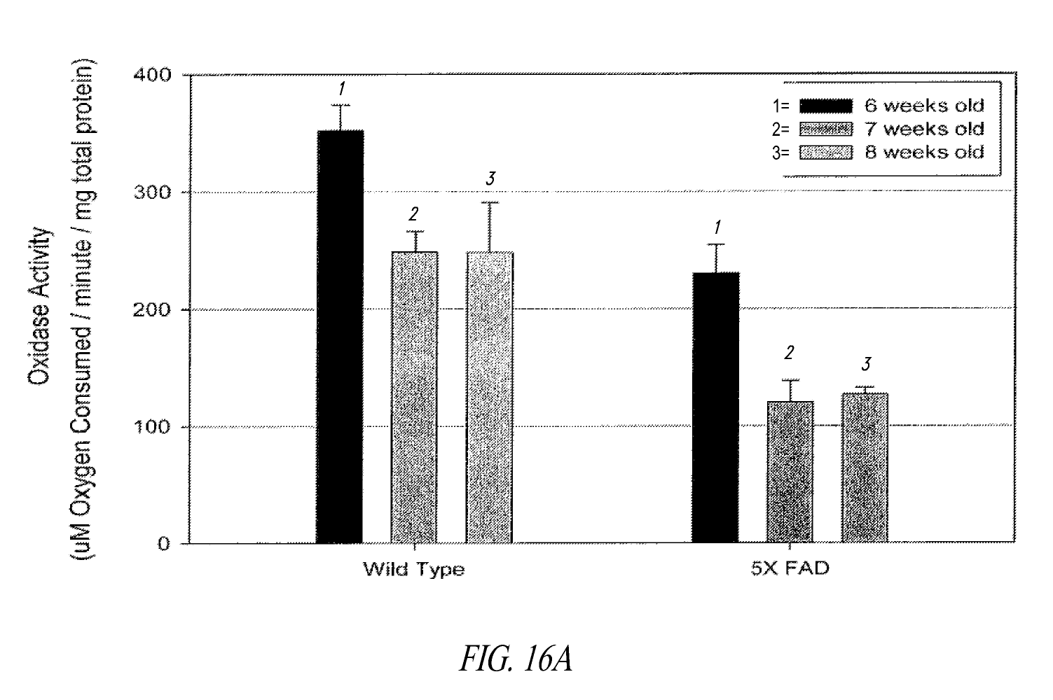

To assess the effects of light on nerve mitochondrial Cco/NO activity we used cerebrum mitochondria, assayed under hypoxic conditions, using ascorbate/TMPD as described above. A 50 watt Xenon/Halogen Floodlight was used as a source of illumination. In order to deliver variable levels of total light energy the light source was placed at different distances from the sample. For these experiments the effects of light was assayed by determining the instantaneous change in rate of NO synthesis observed in the presence of light, relative the rate prior to light exposure. FIG. 14 shows that light stimulates Cco/NO activity in a dose-dependent fashion up to an intensity of 4 mW/cm². Higher intensities than this are less stimulatory. (However, it is important to note the level of stimulation is inversely related to the protein concentration in the chamber, suggesting that higher protein concentrations are capable of defecting some of the light). To assess the effects of specific wavelengths of light on Cco/NO activity a set of broadband interference filters that peak transmittance every 50 nm (between 400 and 700 nm) and a full width half maximum bandwidth (FWHM) of 50 nm were used. An interference filter with a peak transmittance of 880 nm and a full width half maximum of 30 nm was also used. The overall rates of nitric oxide production are shown in FIG. 15. This figure shows that wavelengths of 400±25 nm, 500±25 nm, 550±25 nm, and 600±25 nm all had significant stimulatory effects while the other wavelengths had little or no effect. These results are similar to those obtained with yeast cells with the exception that none of the wavelengths used for mouse mitochondria were inhibitory and while 500 nm light stimulated mouse mitochondrial NO synthesis it had little or no effect on yeast cell mitochondrial NO synthesis.

### Aim 4: NO Synthesis by Mouse Cerebrum Mitochondrial Cytochrome c Oxidase in a Mouse Model for Alzheimer's Disease.

These experiments sought to determine if cerebrum Cco/NO activity is altered in a mouse model for Alzheimer's disease, and 2) to determine if light stimulates cerebrum Cco/NO activity. The mouse model chosen was a Swedish model ‘5×FAD’ transgenic mouse which overexpresses both human APP with 4 Familial Alzheimer's disease point mutations (K670N, M671 L, 1716V, and V7171) as well as human presenelin I (PSI) with two Familial Alzheimer's disease mutations M146L and L286V). The mice have a high level of APP expression and accelerated accumulation of Abeta-42 (the 42 amino acid peptide processed from the amyloid precursor protein). Abeta-42 accumulation starts at 6 weeks of age, prior to amyloid disposition, which begins at 8 weeks of age. These mice have many of the characteristics that are found in human Alzheimer's disease and are considered to be useful models for Abeta-42 induced neurodegeneration as well as amyloid plaque formation. Black 6 (B6) mice, which do not carry the Alzheimer's mutations, were used as controls.

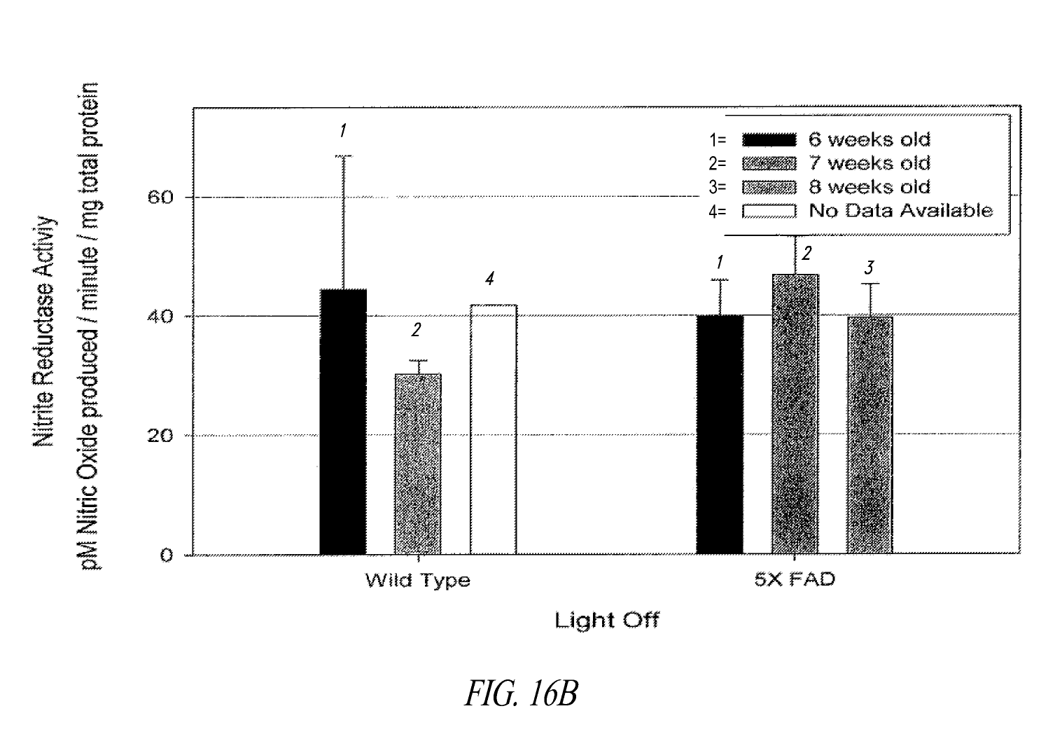

Both the Cco/oxidase and Cco/NO activities in mitochondria isolated from the cerebrums of mice between 6 and 8 weeks old were measured. Both Cco/oxidase and Cco/NO activity was measured using the ascorbate/TMPD pair as electron donors. The data in FIG. 16 reveal that Cco/oxidase activity declines in cerebrum mitochondria taken from both control and 5×FAD mice between the age of 6 and 8 weeks. Interestingly, the level of Cco/oxidase activity was lower in 5×FAD mouse relative to the control at each age. At 6 weeks, the level of Cco/oxidase activity in the 5×FAD mitochondria was 65% of the control but at 7 and 8 weeks it had dropped to 50%. In contrast to Cco/oxidase, Cco/NO activity did not decline with age, in either the control or 5×FAD mice. Moreover, the level of this activity was essentially the same in control and 5×FAD mice.

Table 3 shows that light stimulates Cco/NO activity in cerebrum mitochondria from both control and 5×FAD mice. The level of stimulation varies from 2.5 to 4 fold and can be observed in 6, 7, and 8 week old mice.

TABLE 3
Light stimulation of Cco/NO activity in control and 5X FAD mice

Cco/NO Activity
(pM NO/minute/rag total protein)

| | Light Off | Light On |
|---|---|---|
| 6 week old | | |
| Wild Type | 44.4 ± 22.4 | 174.6 ± 4.4 |
| 5X FAD | 39.8 ± 6.1 | 163.7 ± 9.8 |
| 7 week old | | |
| Wild Type | 30.2 ± 2.3 | 112.8 ± 15.2 |
| 5X FAD | 46.8 ± 6.5 | 134.8 ± 9.7 |
| 8 week old | | |
| Wild Type | Data not available | Data not available |
| 5X FAD | 39.5 ± 5.6 | 91.3 ± 2.8 |

These results clearly support the conclusion that mammalian cells possess Cco/NO activity and that this activity is stimulated by light. Broadband light affects Cco/NO activity in both human endothelial cells and murine mitochondria. It stimulates Cco/NO activity in murine brain mitochondria in a dose and wavelength-dependent fashion. Light is also capable of stimulating Coo/NO activity in murine brain tissue mitochondria from a transgenic mouse model for Alzheimer's disease. Together, these findings provide a conceptual basis for developing light therapy as a tool for treating Alzheimer's disease and other dementias.

### Example 12

### Both Fine and H202 Increase App Processing to ABL-42 which is Reversible Upon Light Treatment

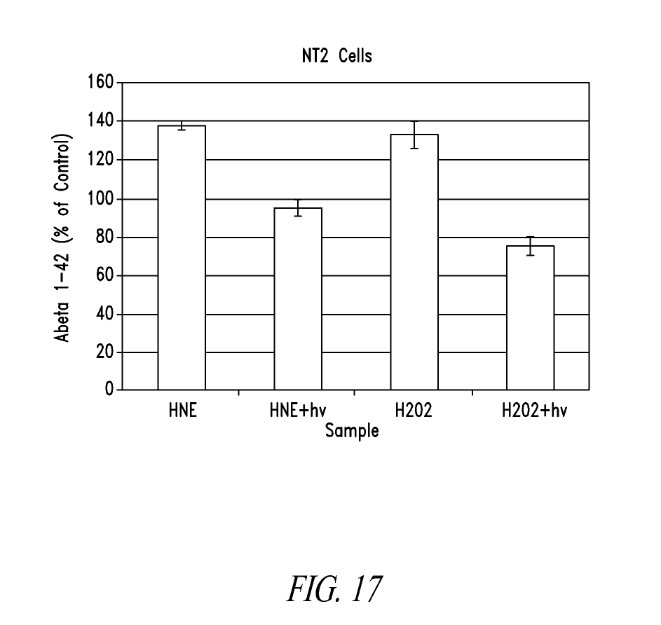

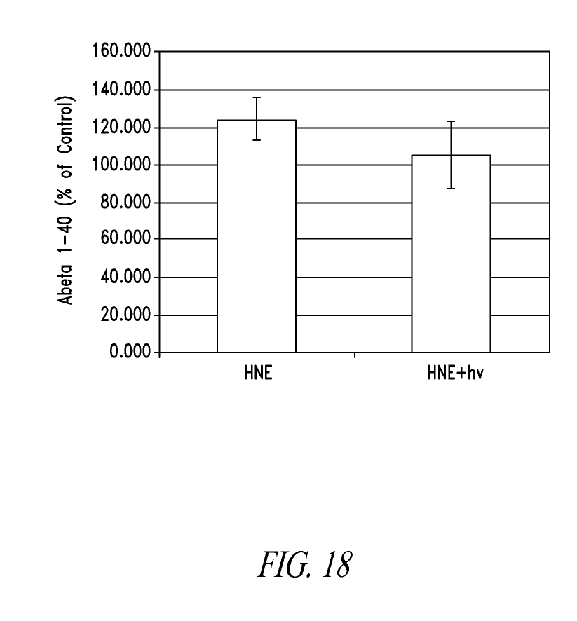

Broadband light reverses oxidative stress induced APP processing to Aβ1-42. NT2 cells treated with either FINE or H2O2 showed dramatic increases in APP processing as assed by Aβ1-42 production. Broad band light (7.5 Joules total dose) reversed the oxidative stress affects back to control levels (FIG. 17).

Broadband light reverses oxidative stress induced APP processing to Aβ1-40. NT2 cells treated with either FINE showed dramatic increases in APP processing as assed by Aβ1-40 production. Broad band light (7.5 Joules total dose) reversed the oxidative stress affects back to control levels (FIG. 18).

These studies clearly demonstrate the ability of light to reverse oxidative stress induced APP processing to both Aβ1-40 and Aβ1-42, the two major peptides responsible for Alzheimer's.

### Example 13

### Evaluation of the Effectiveness of a Light Emitting Device in Enhancing Cognition in Aged Beagle Dogs

The objective of the study was to examine the effectiveness of a light-emitting medical device in improving performance on a short-term working memory task in aged beagle dogs. The underlying rationale was based on evidence that light can be used to activate a light sensitive mitochondrial receptor that mediates the synthesis of nitric oxide (NO). The NO is believed to increase blood flow and improve brain oxygenation, which should improve brain perfusion. Aged dogs, like aged humans and humans with Alzheimer's disease, show decreased brain blood flow, and this is associated with the development of cognitive dysfunction.

Thirty-two (32) study subjects were selected from a pool of 35 dogs aged 9.56 to 15.5 years of age based on baseline performance: The poorest performing animals were selected to maximize baseline levels of cognitive dysfunction. They were then assigned to 4 cognitively equivalent treatment groups of 8 animals per group, which included low, medium and high dose groups and a control group that was connected to the test apparatus, but did not receive the light stimulus. Subjects were evaluated for performance on a delayed-non-matching-to-position (DNMP) test at low, medium and long delays. Testing was in 4 test blocks. Each block consisted on 5 successive days the treatment was applied and 3 successive test days. The treatment day was also the first test day, and occurred 1 hour following administration of the light stimulus. No treatment was applied on either of the next 2 test days.

Thirty-one animals completed the four test blocks. The results varied as a function of test block, application of light-stimulus and dose. On the first two test blocks, there were statistically significant effects of treatment due to improved performance on the session associated with the light therapy treatment. Further analysis revealed that this effect was largely driven by improved performance of the low and medium dose groups, and that improvement was seen at all delays. However, this effect was not observed on the third and fourth test block. These results suggest that the light-therapy has performance enhancing effects and that these effects are transient.

The second experiment looked at the effect of the phototherapy on performance of an attention task and on the DNMP. The same subjects and grouping were used as were used in Experiment 1, except for one animal from the high dose group who had to be dropped because of illness. In this experiment, all subjects were tested twice a day, over a baseline and test phase, which went on 14 consecutive days. The animals received the light therapy treatment once per day. At one hour following the treatment, they were tested on either the DNMP or an attention task protocol. At three hours post-treatment, they were tested on the second task. Thus, if they tested at one hour post-treatment on the DNMP, at three hours post-treatment, they were tested on the attention task. For each task, the post-treatment interval alternated between test days. Thus, if an animal was test first on the DNMP at one hour post-treatment, on following day it would be tested on the DNMP at 3 hours post-treatment. On the baseline phase, the subjects were tested daily over 5 days on the DNMP and daily on a two choice discrimination learning task.

On the attention task, the animals were tested daily over the first 7 sessions with a positive stimulus (which was the same used in 2-choice discrimination) and either 1, 2 or 3 replicates of a negative stimulus. On the same version of the attention task, the negative stimulus was the same one used in 2-choice discrimination. In the different version, a new negative stimulus was used.

As expected, performance on the attention task varied as function of number of distractors: the greater the number, the poorer the performance. We also found that the different version was more difficult than the same, and that performance improved with repeated testing. Examination of the effect of the test treatment suggested improved performance in animals treated with the low and medium dose. The low dose group performed significantly better than the controls when tested with 2 distractors at one hour following treatment on the “Same” task. The medium dose group performed significantly better than the controls at one hour with 3 distractors on the “Same” task, and at 3 hours with I distractor on the “Different” task.

On the DNMP, the low and medium dose animals showing improved performance at the longest delay, when compared to their baseline performance. The medium dose group also showed the best overall performance.

Overall, these results suggest that light therapy can have positive effects on cognition in aged beagle dogs in tests measuring both working memory and attentional processing.

### Study Design

The original study design was a combination of within and between subject design and was used to compare three different dose levels of the treatment variable with control. This entailed selecting 32 aged dogs and assigning them to four cognitively equivalent groups, based on 5 baseline sessions on the delayed-non-matching to position task (DNMP). There were four groups of 8 animals per group.

Once treatment began, the subjects received 4 blocks of testing with 3 sessions in each block. Light treatment was performed on Days 0 to 4, 7 to 11, 14 to 18 and 21 to 25.

At the conclusion of the original study design (Study Day 27), the animals entered a wash-out period. During this time, a protocol addendum was designed to gather further information from the study subjects. As part of the addendum, the subjects received a block of baseline testing in which the animals were tested on 5 sessions of DNMP and a two choice discrimination task up to a maximum of 150 trials (a maximum of 30 trials per day) or until they obtain a two stage criterion (Days 47 to 51). Following this baseline testing, all animals moved onto treatment block 5, where they were tested daily on 18 trials of DNMP (5, 55, 105 second delay) and 16 trials on the Attention task. The DNMP and Attention testing alternated between 1 hour (+/−15 minutes) post dose testing and 3 hours (+/−15 minutes) post dose testing every day, so that each task occurred exactly 7 times at each post dose period. A summary of the study design and addendum study design is provided in FIG. 35.

### 1. Investigational Device

i. Dosage form
   Light of 600 nm wavelength and maximum output of 40 mW/cm²
ii. Doses tested
   Low Dose=1 minute of light treatment
   Medium Dose=10 minutes of light treatment
   High Dose=20 minutes of light treatment
   Control=20 minutes of sham treatment

### Selection and Allocation of Animals

Selection was based on low level of performance and reliability of responding on the DNMP task. Group placement was done in such a way that baseline levels of all groups were equivalent.

### Administration of Test Article/Device

Light was administered via the light-emitting device located on an animal collar. Following baseline blood draws on Day −1, all 32 animals selected to enter the study were sedated with domitor and butorphenol. An ultrasound was used to locate their carotid arteries, and this area was shaved. The animals were tattooed at the location of the carotid artery.

For the treatment, the collar device was placed around the neck and the person restraining the dog ensured that the light-emitting diodes (LEDs) remained over the carotids. The collars were fastened so that there was no folding of skin under the LEDs. A pressure cuff was placed through the collar and was then inflated to 40 mmHg. The purpose of the cuff was to prevent the collar from slipping and to keep the LEDs pressed against the skin so that there was no loss of light. The degree of restraint was minimal. Specifically, the restrainer either had the animal next to him/her and kept a hand on the dog or allowed the dog to rest in a vari-kennel, ensuring that the dog stayed still and that the collar device did not rotate around the neck. On all treatment days, control animals also underwent restraint and a sham device was applied that had the light blocked out.

There were control, low, medium and high dose groups. On days that both treatment and testing occurred, treatment was administered 1 hour (+/−15 minutes) prior to testing. On all other treatment days, light was administered to each subject I hour (+/−15 minutes) from their scheduled testing times. Animals were tested between the hours of 8 AM and 6 PM. Throughout the study each individual animal was tested at the same time fqr every testing day (+/−30 minutes).

A pressure cuff was used on dogs during the administration of the light treatment to ensure that all the collars were secured to each animal at the same pressure. Also, the depth of the carotid artery was measured once via the use of the ultrasound and recorded appropriately during tattooing. The pressure cuff was added to ensure that each LED was pressing onto the skin to the same degree in all dogs to reduce depth-of-penetration variability. The depth of the carotid artery was required to see how deep the light needed to penetrate to reach the artery, which could influence the study results.

### Procedures and Data Recorded

Variable Delay Non-Matching to Position (varDNMP)

DNMP testing was performed as follows. Each trial began with an initial sample presentation of a single block baited with a food reward. This was followed by a delay and a second presentation with two identical blocks: a baited block in the original position covering the empty well and a non-baited block covering the reward in a novel position. The dog was rewarded for displacing the stimulus in the novel position. This study used the variable-delay paradigm in which delays of 5 seconds, 55 seconds and 105 seconds occurred equally over 18 test trials per day, resulting in 6 trials for each delay. The delays occurred randomly within the test session and each possible position was used for each delay.

Dogs were tested for 5 baseline days, and for 3 days per test block. Animal test times were kept to the same time each day; including days where treatment and testing occurred on the same day.

Following Test Block 4, all dogs completed another 5 baseline days, followed by 14 days of treatment and testing occurring on the same day, with the DNMP task alternating every other day between 1 hour (+/−15 minutes) and 3 hours (+/−15 minutes) post dose.

Attention Task

Attention task testing was carried out on the days indicated in Section 6.1 Study Design Summary. Testing was performed as per SOP DOG.38.01, with the following protocol specific instructions.

There were 30 trials per session during 2-choice discrimination (object pair presentation) and there was one test session per day for all subjects during baseline testing.

During true Attention testing (in which 1-4 objects were presented), which occurred during the test phase (14 days), animals were tested on 16 trials per test session. For each test session, the preferred object occurred exactly 4 times within each location on the test tray. There was no criterion for the attention cognitive task.

There were a number of deviations made on the Attention task. For instance, one deviation consisted of an animal receiving two additional trials on the preference test, and another consisted of an animal receiving an single additional trial on the true attention task. On Day 48, two animals were only tested on 20 consecutive trials of 2-choice acquisition instead of 30 trials. As well, on Day 59, four animals were tested with the incorrect positive and negative stimuli during Session 8 of the true attention task.

### Blinding of the Study

During study days 0 to 27, the treatment given to each animal was not revealed to the technicians involved with data collection. The study was blinded to all personnel with the exception of the persons involved in administering the investigational light-emitting medical device and the persons responsible for performing and verifying allocation. Those people did not collect data other than at the time of treatment.

The personnel performing the cognitive tests, the Study Director and the Study Monitor were blinded to ensure an unbiased assessment of performance.

For all procedures conducted under the protocol addendum, research technicians conducting daily observations and those responsible for performing the cognitive testing remained blinded. All other study personnel were unblinded to treatment assignment.

### Calculations and Statistical Analyses

The data were analyzed with both analysis of variance and with one-tailed t-tests, with the expected result being superior performance by the animals in the treatment groups.

Results: Experiment 1

Baseline Cognitive Characterization

Although there were originally 8 animals per group, one of the animals assigned to the high dose group did not enter the study because of the development of severe movement difficult diagnosed as intravertebral disc disease (IVDD). Thus, there were 8 animals in three of the groups and 7 animals in the fourth.

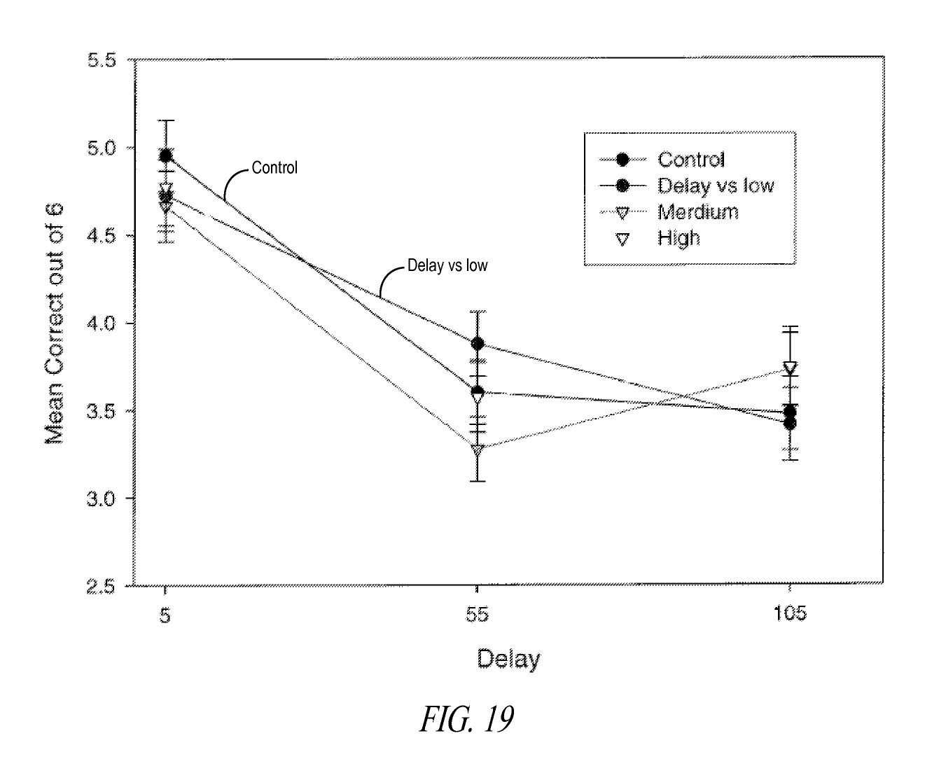

To verify that the groups were cognitively equivalent at baseline, the average number of daily correct responses at each delay were calculated and the grouped baseline data were compared using a repeated measures ANOVA with delay as a within subject variable and Grouping as a between subject variable. The ANOVA revealed a statistically significant effect of delay (p=0.00) and no other significant effects or interactions. As illustrated in FIG. 19, the delay effect resulted primarily from more accurate performance at the 5 second delay, when compared to 55 or 105. Multiple comparisons using Tukey revealed statistically significant differences between 5 and 55 and between 5 and 105, but no differences between 55 and 105.

### Treatment Phase: Performance on DNMP as a Function of Test Block, Treatment Day and Dose

The subjects received a 19 day washout period in which no data was collected on the animals. A 5 day baseline testing period followed which consisted of a delayed-non-matching to position task and an attention task. The four groups with 8 animals per group remained the same. The subjects then received an additional 14 days of testing, with treatment occurring on all 14 days 1 hour (+/−15 minutes) prior to their first testing session of the day. Testing consisted of the DNMP task (Memory) as well as the Attention task (Learning). DNMP and Attention testing alternated between 1 hour (+/−15 minutes) post dose testing and 3 hours (+/−15 minutes) post dose testing every day, so that each task occurs exactly 7 times at each post dose period.

The Attention task testing was performed with the following protocol specific instructions. There were 30 trials per attention test session (during object pair testing) and there was one test session per day for all subjects during baseline testing. During true Attention testing (in which 1-4 objects will be presented), which occurred during the test phase, animals were tested on 16 trials per Attention test session. For each test session, the preferred object occurred exactly 4 times within each location on the test tray.

Additional cognitive data was desirable to establish the immediate effectiveness of the medical light device on cognition. The repeated post dose testing allowed us to confirm whether the device was effective if testing occurred after a short post dose interval. The introduction of the attention task provided additional data about cognitive effectiveness in another cognitive domain, that of selective attention. The additional data collected from the performance of this addendum increased the statistical power of the data previously collected, making it more likely to see a positive effect if the device is actually effective.

These analysis first looked at performance over the entire experiment, which revealed that the effectiveness of the treatment varied as a function of test block. Further analysis indicated that performance of the low and medium groups improved under the treatment condition on the first two test blocks, and that this improvement was statistically significant when compare with average of preceding and following test sessions for the medium dose group at the 5 and 55 second delays on the first treatment session. On the second treatment session, both the low and medium dose groups showed statistically significant improvement at each delay.

The treatment phase consisted of four test blocks with each block consisting of 5 treatment days and 3 consecutive daily test sessions. The first was approximately one hour following either the last phototherapy or control treatment. The second was approximately 24 hours following the first session and the third was 36 hours following the first session.

The data were first analyzed with omnibus ANOVA over 4 test blocks and baseline. Group (control, low dose, med dose, and high dose) served as between subject variable. Within subject variables were test block (control, test block 1, test block 2, test block 3 and test block 4), treatment day (test following treatment, post treatment day 1, and post treatment day 2) and delay (5,55,105). The results revealed a significant effect of delay (p=0.000), a significant test block by treatment interaction (p=0.0029), and a significant treatment by delay interaction (p=0.029). There were no significant group effects. However, the treatment by Group effect was marginally significant (p=0.130).

The significant test block by treatment interaction, reflected the fact that he effect of the treatment varied as a function of test blocks. To better understand this effect, we then looked at each test block separately and also included the last day of the previous block. Thus, the analysis for test Block I was based on four test days—the last baseline day, the treatment day, post treatment day I and post treatment day 2. The analysis for test block 2 was also based on four test sessions, the first of which was the last test day of test Block 1.

Block 1.

At each test block, the data were analyzed with repeated measures ANOVA with delay treatment session and delay as within subject variables and test group as a between subject variable. The results revealed a statistically significant effect of delay (p=0.000) and treatment day (p=0.0016). The delay effect reflected, as expected, accuracy decreasing with decreasing delay. The comparisons between 5 S and both 55 and 105 were statistically significant. The treatment day effect was due to the groups (independently of treatment level) performing better on day 2 than the other 3 days, with day 2 being the day that the animals were given light therapy.

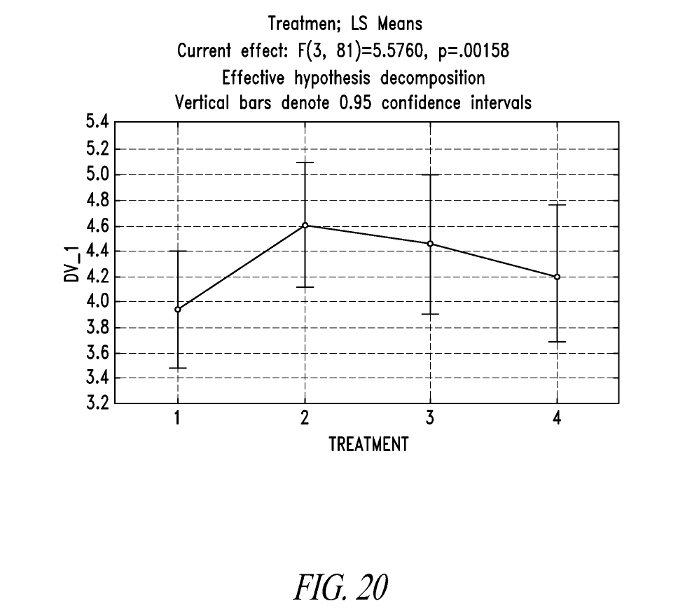

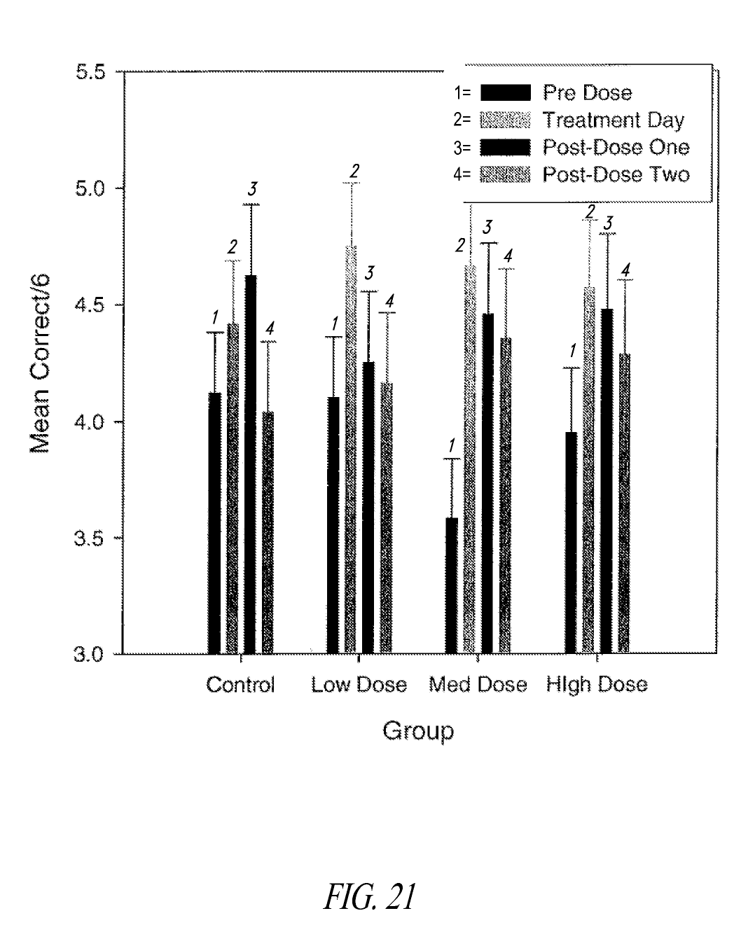

Although, as shown in FIG. 20, the treatment effect is based on an average of all groups, the day 2 effect (which reflects performance following light therapy treatment) was driven by performance of the low and medium dose groups and to a lesser extent, the high dose group (see FIG. 21). Thus, these results specifically suggest that the therapy produces a short-lasting facilitation of DNMP performance.

To further analyze the effect of the light treatment, for each group at each delay, the mean performance on day 1 and day 3 was compared with performance of the same group on day 2, following treatment using one-tailed t-test for paired samples. Statistically significant differences were found for the medium dose group at the 5 second delay (p=0.047) and 55 sec delay (p=0.044), indicating that the medium dose level was most effective.

Block 2.

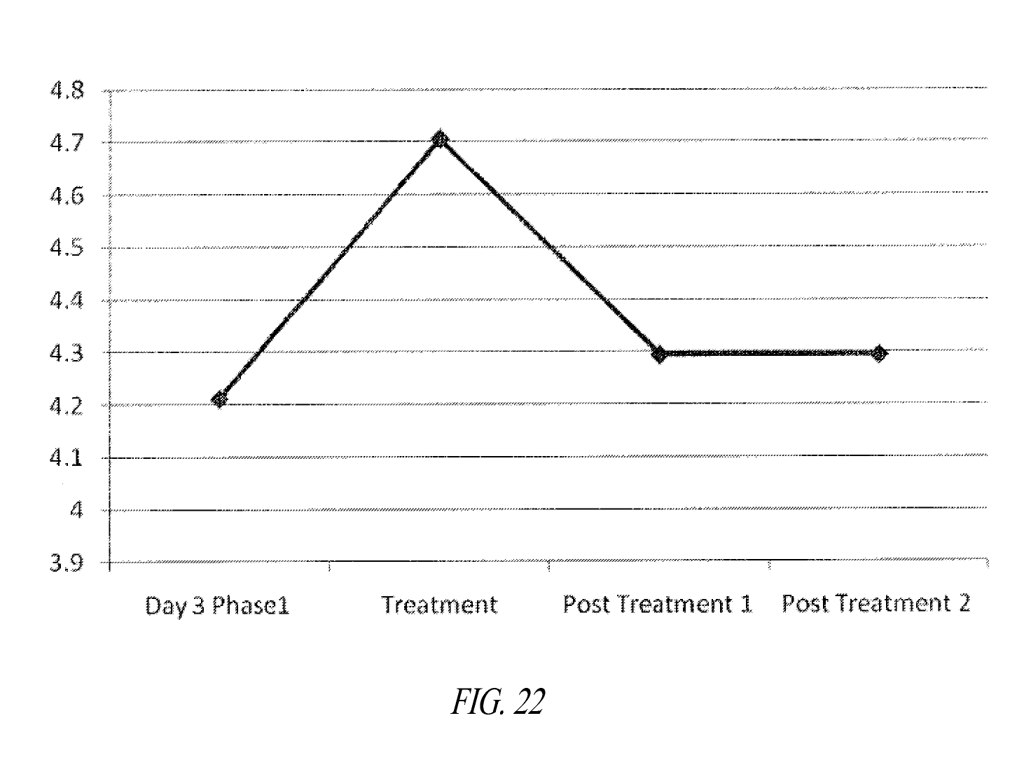

The results for test-block 2 were analyzed using a repeated measures ANOVA, with the first test day being the fourth test day of block 1. The results of the ANOVA revealed a statistically significant main effects of treatment (p=0.014) and delay (p=0.00) and a significant one way interaction between treatment and Group (p=0.022). There was also a marginally significant interaction between Treatment, Delay and Group (p=0.103). The origins of the treatment day effect are shown in FIG. 22, which illustrates that the groups responded more accurately on the treatment day.

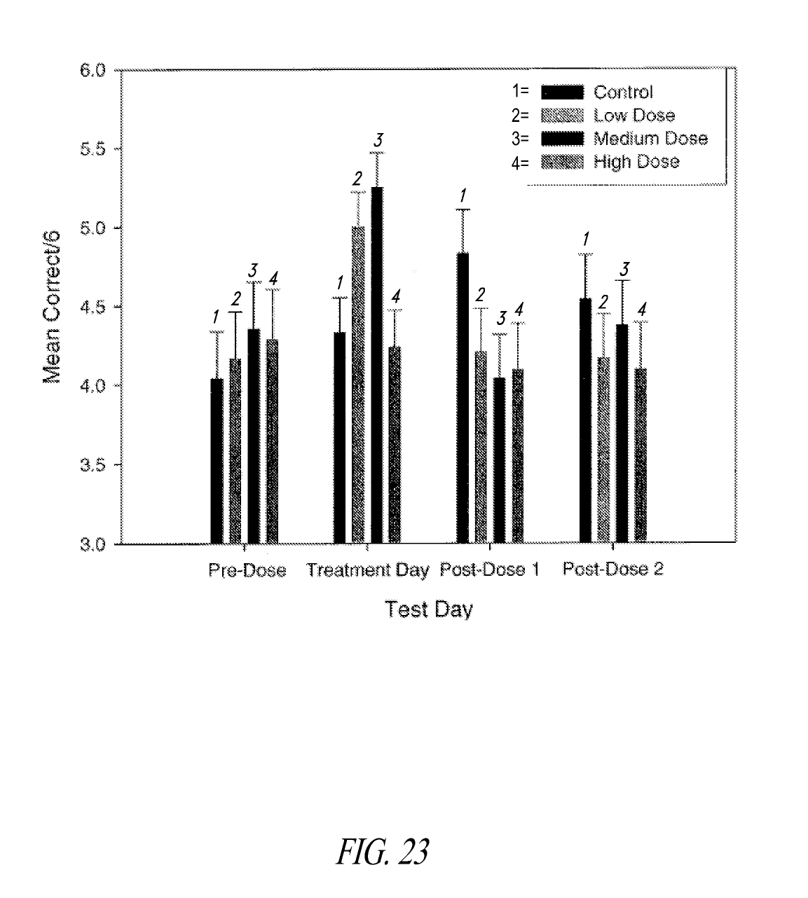

FIG. 22 illustrates that the treatment day effect was similar to that seen in test Block 1, and was largely due to more accurate performance during treatment session 2. FIG. 23 illustrates that the treatment by group interaction reflected the low and medium dose animals performing maximally on treatment day 2, while the controls showed best overall performance on treatment day 3.

There was a clear negative correlation between accuracy and delay, with performance and 5 seconds differing significantly from performance at the two higher delays.

To further analyze the effect of the light treatment, for each group at each delay, the mean performance on day 1 and day 3 was compared with performance of the same group on day 2, following treatment using one-tailed t-test for paired samples. Statistically significant differences were found for the low dose level at all delays (5 S, p=0.035; 55 S, p=0.015; 105 S, p=0.046). The medium dose group also showed statistically significant effects at all delays 5 S, p=0.001; 55 S, p=0.026; 105 S, p=0.016). There were no significant differences found for the high dose group or control group.

Block 3.

The analysis of variance for block 3 showed a significant main effect of delay (p=0.000). Unlike block 1 and block 2, there were no other significant main effects or interactions.

There was a clear negative correlation between accuracy and delay, with performance and 5 seconds differing significantly from performance at the two higher delays.

To further analyze the effect of the light treatment, for each group at each delay, the mean performance on day 1 and day 3 was compared with performance of the same group on day 2, following treatment using one-tailed t-test for paired samples. There were no statistically significant effects of the light-treatment. The controls animals showed a significant effect at the long delay (p=0.009).

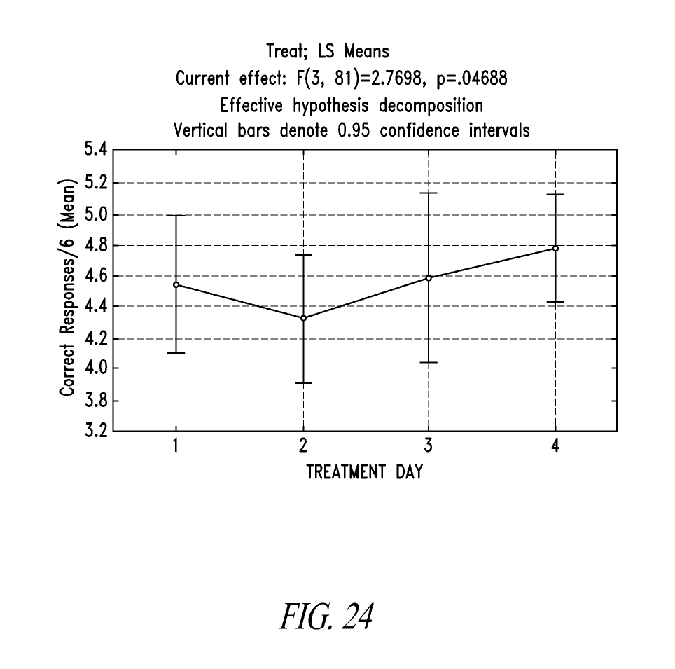

Block 4. The analysis of variance results for test block 4 revealed significant main effects of treatment (p=0.047) delay (p=0.000) and no other significant main effects or interactions. FIG. 24 illustrates that the main effect of treatment reflected poorer overall performance on the day the subjects were given the light therapy.

There was a clear negative correlation between accuracy and delay, with performance and 5 seconds differing significantly from performance at 55 and 105.

Results: Addendum (Experiment 2)

Acquisition of Two-Choice Discrimination.

The animals were trained on the 2-Choice discrimination learning task during baseline, prior to testing on the attention task. All but three animals successfully completed the two stage learning criterion within the 150 trails allotted. Two of the remaining three had completed the first phase and one trial of the second phase. In both cases, the animals' response was above the 70% on the second phase, so it was deemed that the animals had learned the task. The one exception had not completed the first phase of the learning.

Note that the animals assigned to the control condition were the highest performing group. This would have led to the prediction that the controls would also be the highest performing group on the attention task.

Number of trials to criterion were analyzed with a one way analysis of variance with total both errors over training sessions and percent correct as dependent variable and group as independent variable. The results revealed a marginally significant effect of errors (p=0.09979) and a statistically significant group effect on percent correct (p=0.019844).

Group comparisons using Fischer LSD method revealed that the control group performed better than the other three, with the differences between control and high dose being statistically significant, while the difference between the control and low dose was marginally significant.

Performance on Attention Task.

The “same” condition used the same stimuli as those used in the two-choice discrimination. Thus, the positive stimulus was always the same. On any given trial, the negative stimulus was either absent (4 trials daily), present in a single replicate (4 trials daily), present in duplicate (4 trials daily) or present in triplicate (4 trials daily).

One animal, that did not learn the two-choice discrimination task was not included in the analysis of the attention task because the attention task required that the animal respond to the stimulus that it had previously learned was associated with reward.

On both the same and different conditions of the attention task, the animals were scored based on percent correct out of total responses attempted at each level of distractor. Thus, if animal responded correctly on 11 trials and did not respond on the 12^{th} it was given a score of 100%.

The animals were tested on 7 sessions, with sessions 1, 3, 5 and 7 occurring five hours following treatment and sessions 2, 4, and 6 occurring one hour following treatment.

Omnibus Anova

The data were first analyzed using Omnibus ANOVA, which was followed by separate repeated measures of ANOVAs were used to compare performance on each of the tasks. This analysis did not reveal any significant differences between the four treatment groups. However, there was an overall trend indicating superior performance by the medium dose group.

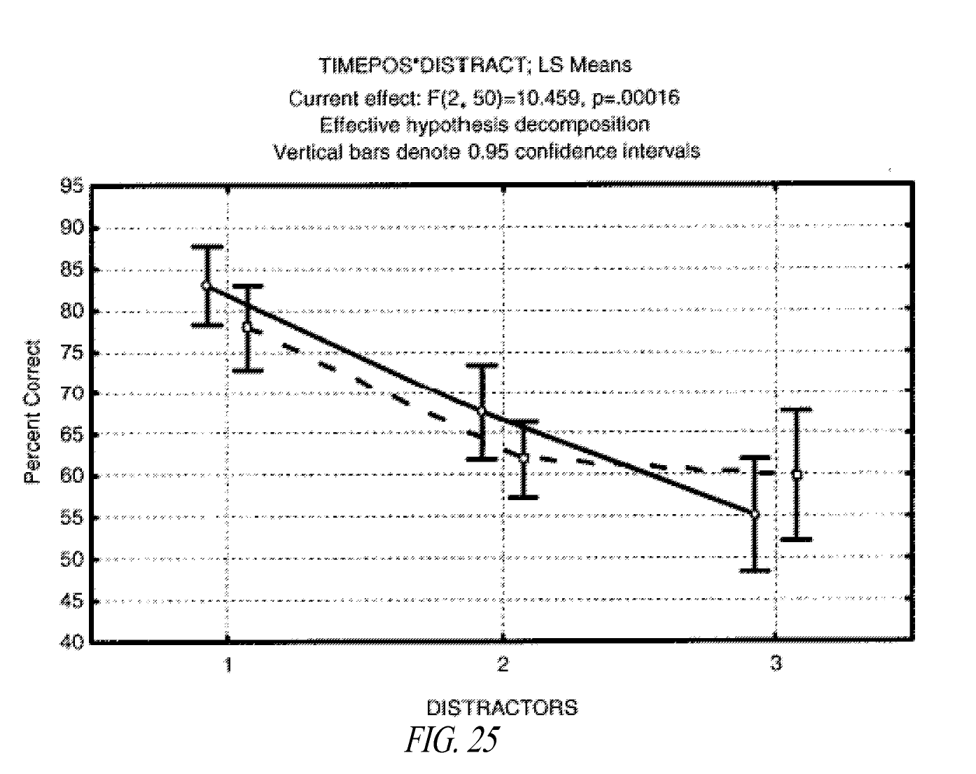

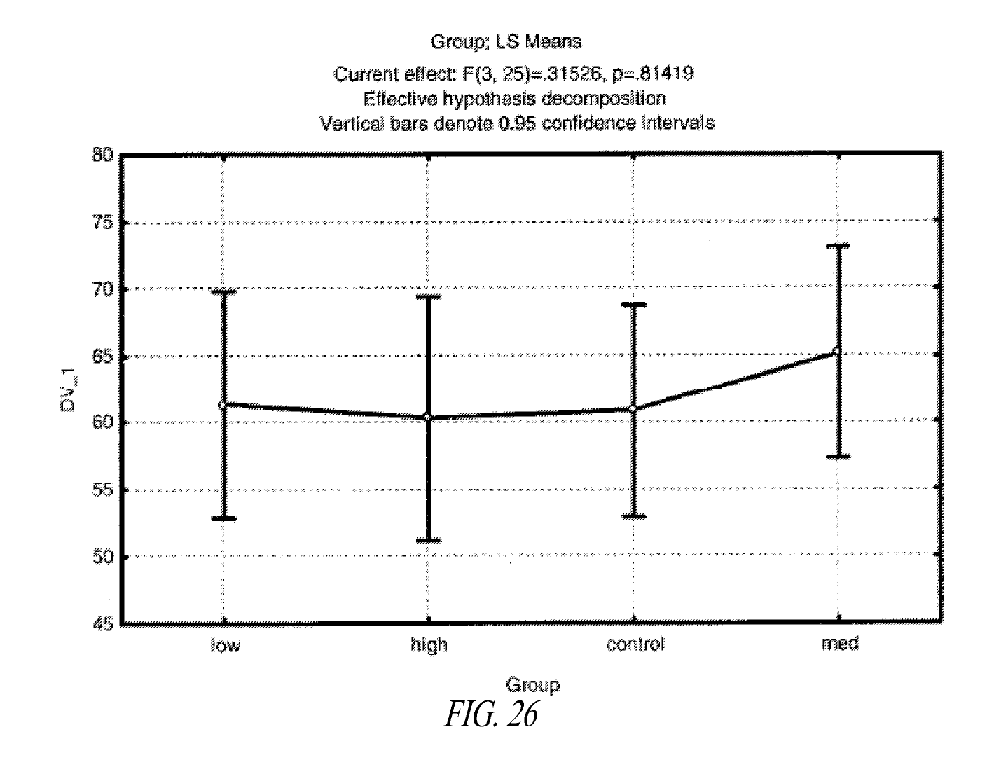

The omnibus ANOVA compared performance of the groups (between subject variable) on the two tasks (same vs different), number of distractors, and time following treatment (1 vs 3 hours), all of which served as within subject variables. The results revealed statistically significant effects of task (p=0.000) and number of distractors (p=0.000). There was also a significant interaction between time post treatment and number of distractors (p=0.000). The task effect reflected, as expected, superior performance on the same task when compared to the different task. FIG. 25 illustrates that the significant effect of distractor reflects decreased accuracy of performance with increased number of distractors. The significant interaction reflects improved performance when tested 3 hours following treatment with three distractors when compared with performance 1 hour following treatment. The significance of this finding is not clear.

Although the groups did not differ significantly, there was a trend towards the low and medium dose groups showing the best overall performance (see FIG. 26).

Same Condition

The next analysis was restricted to performance on the same version of the task. The results did not reveal any statistically significant group effects, but there were clear trends showing improved performance of the medium and low dose groups, when compared to the controls.

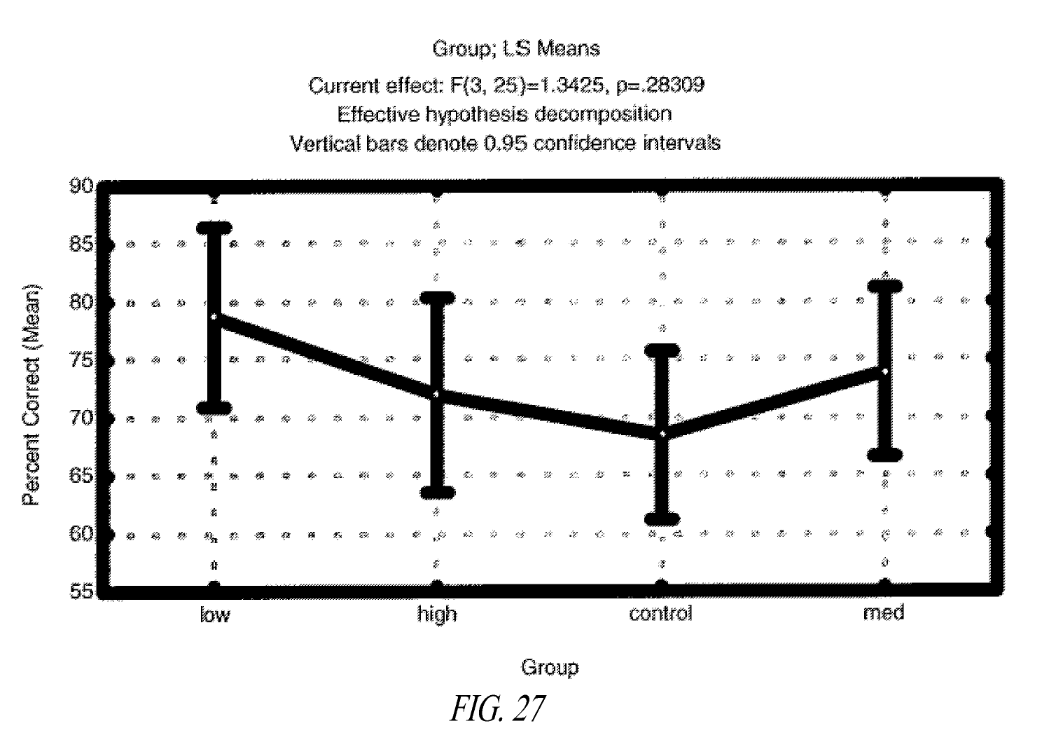

The data from the same task were analyzed with a repeated measures ANOVA with group as within subject variable and time post treatment and distractors as between subject variables. There were no significant main effects, but there was a significant interaction between time following treatment and number of distractors (p=0.036) reflecting better performance at three distractors when tested two hours following treatment (see FIG. 25). Although the groups did not differ significantly, the there was trend towards the low and medium dose groups showing the best overall performance (see FIG. 27).

Different Condition

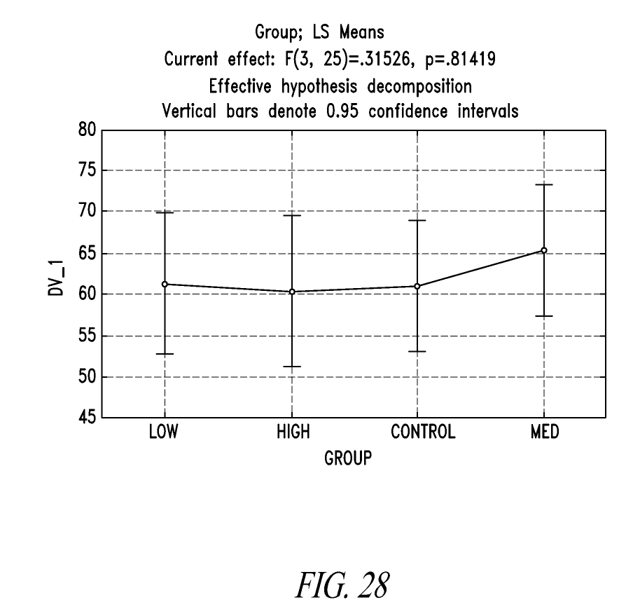

The data from the different task were analyzed with a repeated measures ANOVA with group as within subject variable and time post treatment and distractors as between subject variables. There were no significant main effects, but there was a significant interaction between time following treatment and number of distractors (p=0.009) reflecting better performance at three distractors when tested two hours following treatment (see FIG. 25). The groups did not differ significantly, but there was trend towards medium dose groups showing the best overall performance (see FIG. 28).

Comparisons Using t-Tests

To provide further data of effectiveness, each treatment group was compared with the controls for each level of distractor and at each post treatment interval using 1-tailed t-tests. The results indicate that the low dose group performed significantly better than the controls when tested with 2 distractors at one hour following treatment on the “Same” task. The differences between the controls and low dose group at 1 hour with three distractors on the “Same” task and at 2 hours with 2 distractors on the “Different” task were marginally significant. The medium dose group performed significantly better than the controls at one hour with 3 distractors on the “Same” task, and at 3 hours with 1 distractor on the “Different” task.

Changes Over Repeated Testing on “Same” and “Different” Task.

A final descriptive analysis was carried out comparing the groups performance on both tasks on daily basis. Although the analyses did not pick up any statistically effects of treatment, the analyses did reveal progressive improvement with repeated testing—a learning effect, and on both tasks, there was a trend for the low and medium dose groups to show better learning.

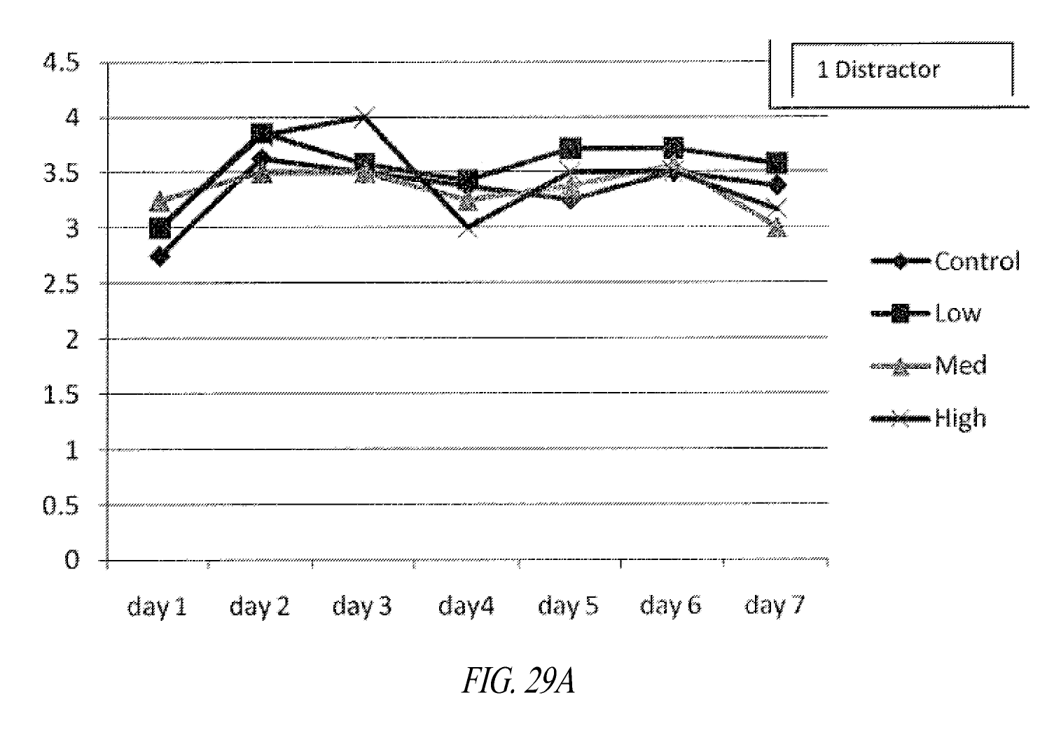

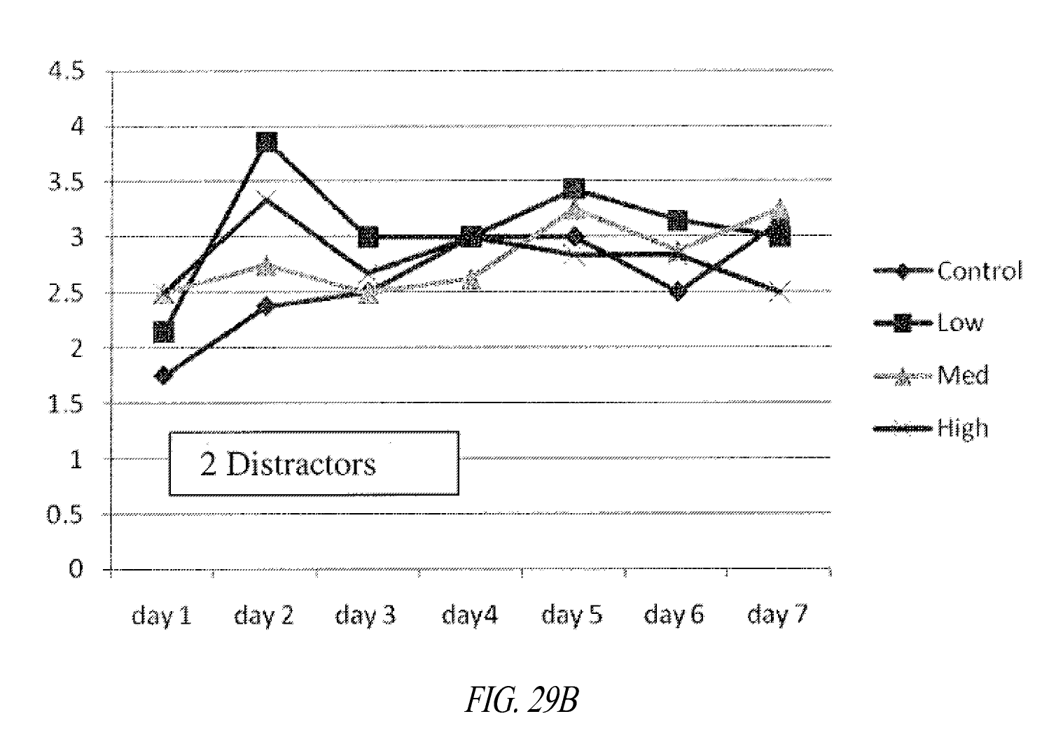

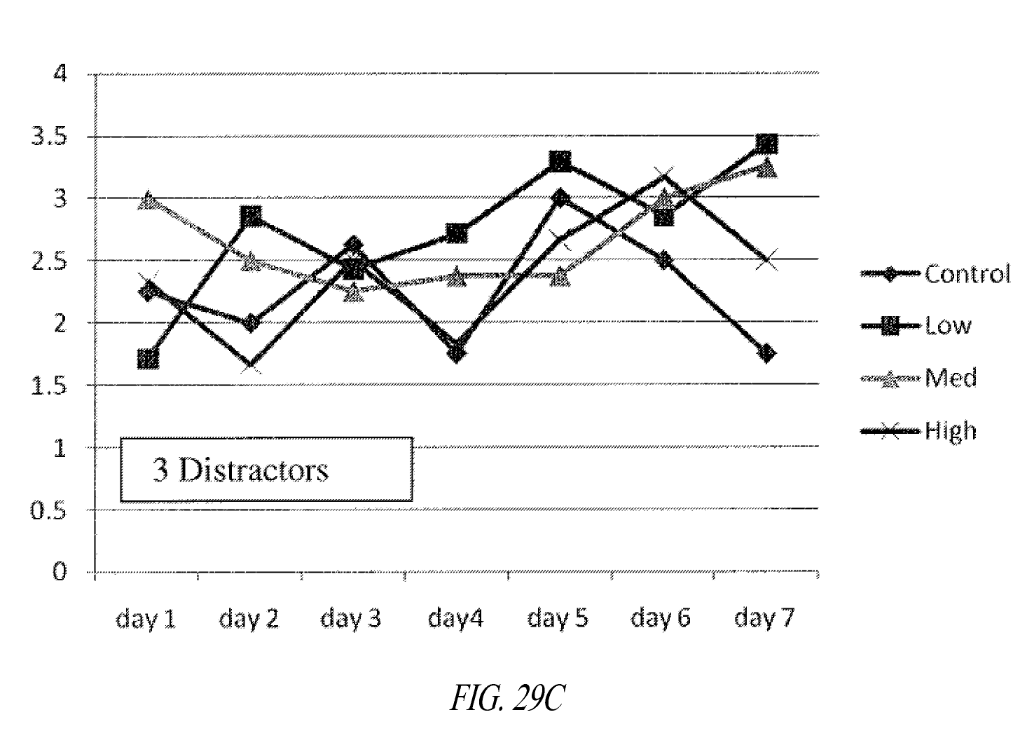

For both tasks, the data were analyzed with an analysis of variance over the 7 day test period with test group as within between subject variable and days and number of distractors as within subject variables. The results of the analysis revealed significant main effects of test day (p=0.044) and number of distractors (p=0.000). There was also a significant interaction between test day and number of distractors (p=0.0169). The origin of these results is shown in FIG. 29. Performance with one distractor was relatively stable through the test interval. With 2 and 3 distractors, overall, the groups showed progressive improvement account for the days effect and the days by distractor interaction.

There were no significant group effects, although on the three distractor condition, the controls tended to show less improvement than any of the treatment groups.

The results of the analysis of the difference task showed that there were significant main effects of test day (p=0.000) and number of distractors (p=0.000). There was also a significant interaction between test day and number of distractors (p=0.0000).

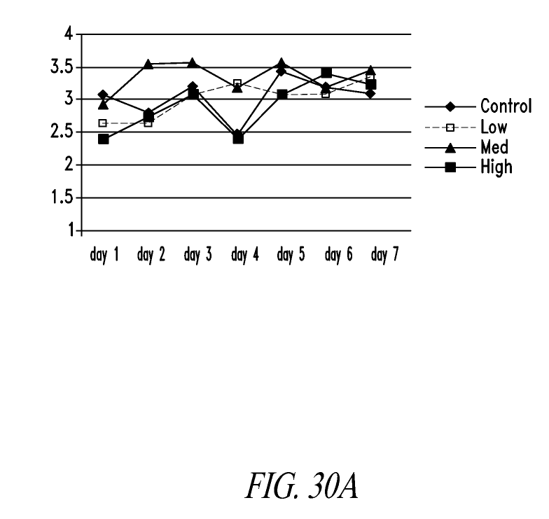

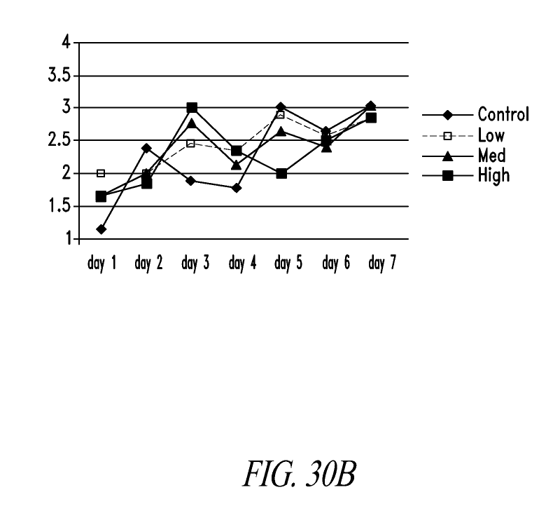

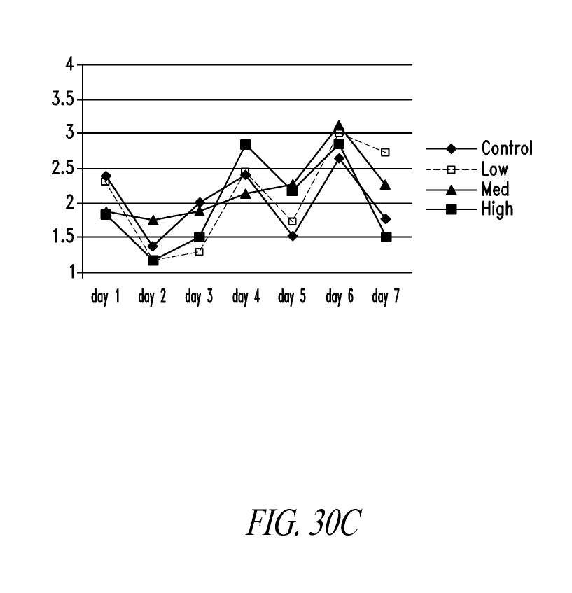

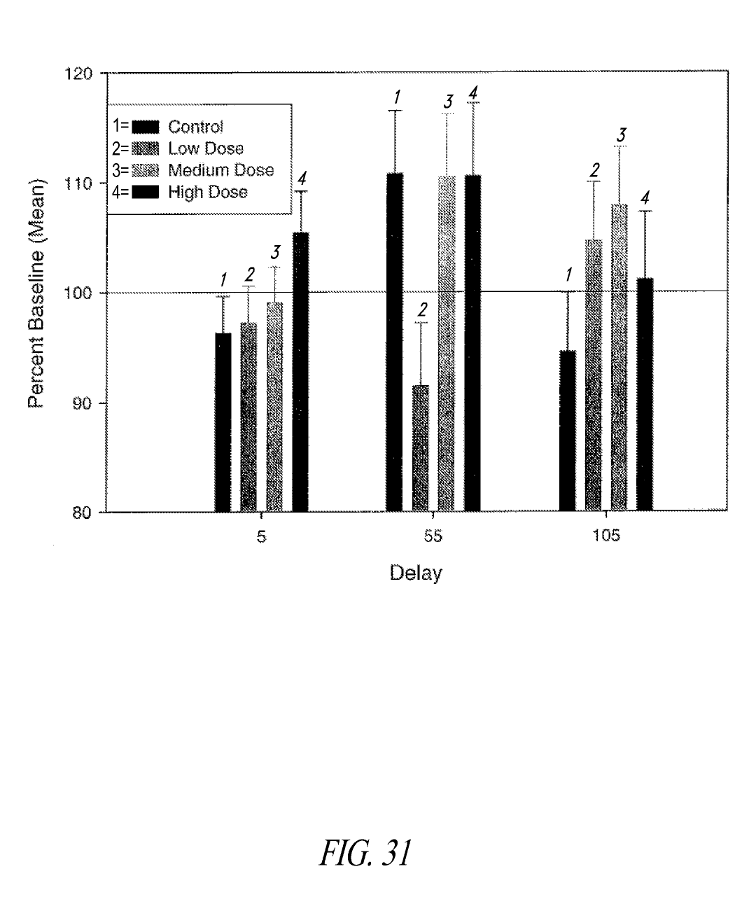

FIG. 30 illustrates that the groups all showed progressive improvement when repeatedly tested with 2 or 3 distractors. Although there were no significant group effects, the low and medium dose groups tended to perform better over repeated testing than the controls or high dose group.

Performance on DNMP

Subjects performance over the 14 test session were compared with baseline, which was calculated by dividing percent correct under treatment condition by percent correct under baseline and multiplying by 100. Non responses were discarded. Thus, if an animal responded on 16 or 18 trials and didn't respond on the other two, it would receive a score of 100. The data were first analyzed with a repeated Measures ANOVA with test Group as a between subject variable and Delay (5, 55, 105), and time following dosing (I or 3 hrs) as within subject variables. The results of the analysis revealed a significant interaction between delay and group (p=0.044) and no other significant main effects or interactions.

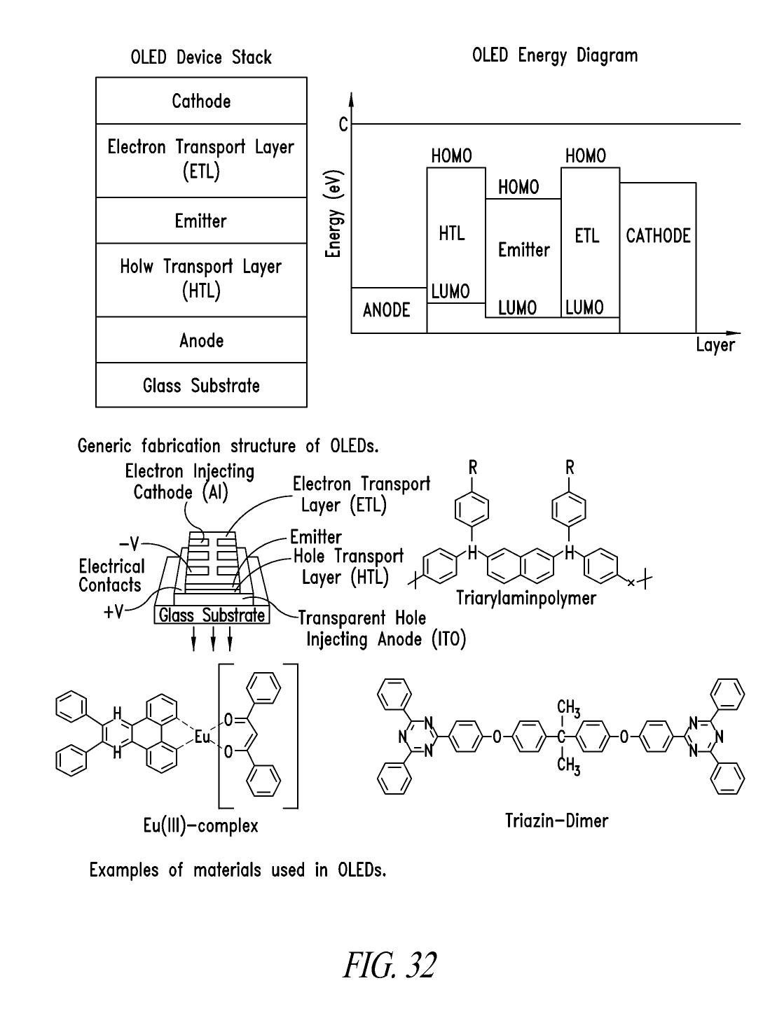

The origins of this are illustrated in FIG. 31, which shows that the low and medium dose animals showing improved performance at long delay. The control group, by contrast, showed poorer performance at long delay, but improved performance at 55 S delay. This figure also illustrates that the medium dose group showed best overall performance.

### DISCUSSION

The purpose of this study was to examine the effectiveness of a form of light therapy on cognitive function in a group of cognitively impaired aged beagle dogs.

The first part of the study examined the effect of a single treatment over 4 successive test blocks, with each block consisting of 5 treatment sessions and 3 test sessions—one of which occurred 1 hour following the 5^{th} treatment session. To analyze the data, we looked at performance over successive blocks of 4 test sessions. Session 1 was the last pre-treatment test session; session 2, the session one hour following the last treatment; session 3, the session 24 hours following the last treatment and session 4, 48 hours following the last treatment. Session one of the first test block was the last baseline test session. Session one of the second test block was also the last session of test-block 2. We used this procedure to allow us to compare performance on the DNMP with the performance on both the preceding and following sessions, with the assumption that this would allow us to control for possible practice effects.

The results varied as a function of test block and dose. On the first two test blocks, there was a statistically significant effect of treatment, which reflected improved performance on the one hour post-treatment test. This effect was not observed on the third and fourth test block. This positive effect seen in the first treatment block was largely driven by improved performance of the low and medium dose groups; and was not seen in the control animals. This conclusion was supported by analysis comparing the animals' performance at each delay on the one hour post-treatment session with that of their mean performance on the preceding and following sessions. On the first treatment block, the medium dose group performed significantly better at both the short and medium delays. On the second treatment block, both the low and medium dose group performed significantly better at all delays. These results suggest at least a transient memory-improving effect of the treatment.

The absence of a treatment effect on the third and fourth test block is not explained. It may reflect a practice effect. According to this explanation, performance may initially improve partly because of practice, eventually reaching a plateau or their maximum performance capabilities. This suggestion was supported by the observation that performance, overall, improved, as a function of test block. Alternatively, the effect of the treatment may diminish with time or by tolerance or be countered by other direct or indirect effects of the treatment.

The second experiment looked at the effect of the phototherapy on performance of an attention task and on the DNMP. The same subjects and grouping were used as were used in Experiment 1, except for one animal from the high dose group who had to be dropped because of illness. In this experiment, all subjects were tested twice a day, over a baseline and test phase, which went on 14 consecutive days. The animals received the phototherapy treatment once per day. At one hour following the treatment, they were tested on either the DNMP or an attention task protocol. At three hours, they were tested on the task the second task. Thus, if they tested at one hour on the DNMP, at three hours they were tested on the attention task. The first task alternated between test days. On the baseline phase, the subjects were tested daily over 5 days on the DNMP task and for up to 5 days on a two-choice discrimination learning task.

There was one potentially important adverse effect of the treatment, namely the development or enhancement of intravertebral disc disorder (IVDD). This was diagnosed in 2 animals during the study, Bombay and Sydney, one of which had to be removed from the study. Six other dogs were evaluated and observed to have neurological deficits consistent with IVDD a week after study completion. Six of these 8 animals were from the control and high dose groups, which were subjected to 20 minutes of either treatment or sham treatment, According to both the clinical veterinarian and veterinary technician, the problem was not the collars but the head position the dogs had to assume while wearing them. The collars may exacerbate IVDD or cause premature decompensation. More specifically it was the pressure coming from the inflation of the pressure cuff around the neck of the dog that was likely causing the decompensation of the animals. These observations suggest that adjustments should be made to the device for future studies, to deal with this issue in the future.

Despite the correlation between treatment and IVDD, there was no obvious relationship to cognitive performance, making it unlikely that the development of IVDD affected the cognitive data.

Overall, these results are consistent with hypothesis that light-therapy can be used as a potential therapeutic for treatment of age-dependent cognitive dysfunction. Thus results also suggest, however, that the beneficial effects are transitory, although they may persist for 3 hours or possibly longer. We found no evidence of the limited testing protocol here producing a more permanent cognitive change after cessation of treatment.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Beal, M. F. 2000. Oxidative metabolism. Ann NY Acad Sci. 924:164-9.
Castello, P. R., P. S. David, T. McClure, Z. Crook, and R. O. Poyton. 2006. Mitochondrial cytochrome oxidase produces nitric oxide under hypoxic conditions: implications for oxygen sensing and hypoxic signaling in eukaryotes. Cell Metab. 3:277-87.
Castello, P. R., D. K. Woo, K. Ball, J. Wojcik, L. Liu, and R. O. Poyton. 2008. Oxygen-regulated isoforms of cytochrome c oxidase have differential effects on its nitric oxide production and on hypoxic signaling. Proc Natl Acad Sci USA. 105:8203-8.
Chandel, N. S., D. S. McClintock, C. E. Feliciano, T. M. Wood, J. A. Melendez, A. M. Rodriguez, and P. T. Schumacker. 2000. Reactive oxygen species generated at mitochondrial complex III stabilize hypoxia-inducible factor-1 alpha during hypoxia: a mechanism of 02 sensing. J Biol Chem. 275:25130-8.
Colton, C. A., M. P. Vitek, D. A. Wink, Q. Xu, V. Cantillana, M. L. Previti, W. E. Van Nostrand, J. B. Weinberg, and H. Dawson. 2006. NO synthase 2 (N052) deletion promotes multiple pathologies in a mouse model of Alzheimer's disease. Proc Natl Acad Sci USA. 103:12867-72.
DeLellis, S. L., D. H. Carnegie, and T. J. Burke. 2005. Improved sensitivity in patients with peripheral neuropathy: effects of monochromatic infrared photo energy. J Am Podiatr Med Assoc. 95:143-7.
Dirmeier, R., K. M. O'Brien, M. Engle, A. Dodd, E. Spears, and R. O. Poyton. 2002. Exposure of yeast cells to anoxia induces transient oxidative stress. Implications for the induction of hypoxic genes. J Biol Chem. 277:34773-84.
Eells, J. T., M. T. Wong-Riley, J. VerHoeve, M. Henry, E. V. Buchman, M. P. Kane, L. J. Gould, R. Das, M. Jett, B. D. Hodgson, D. Margolis, and H. T. Whelan. 2004. Mitochondrial signal transduction in accelerated wound and retinal healing by near-infrared light therapy. Mitochondrion. 4:559-67.
Grishko, V., M. Solomon, J. F. Breit, D. W. Killilea, S. P. Ledoux, G. L. Wilson, and M. N. Gillespie. 2001. Hypoxia promotes oxidative base modifications in the pulmonary artery endothelial cell VEGF gene. Faseb J 15:1267-9.
Harkless, L. B., S. DeLellis, D. H. Carnegie, and T. J. Burke. 2006. Improved foot sensitivity and pain reduction in patients with peripheral neuropathy after treatment with monochromatic infrared photo energy—MIRE. J Diabetes Complications. 20:81-7.
Karu, T. 1999. Primary and secondary mechanisms of action of visible to near-IR radiation on cells. J Photochem Photobiol B. 49:1-17.
Karu, T. I., L. V. Pyatibrat, S. F. Kolyakov, and N. I. Afanasyeva. 2005. Absorption measurements of a cell monolayer relevant to phototherapy: reduction of cytochrome c oxidase under near IR radiation. J Photochem Photobiol B. 81:98-106.
Oakley, H., S. L. Cole, S. Logan, E. Maus, P. Shao, J. Craft, A. Guillozet-Bongaarts, M. Ohno, J. Disterhoft, L. Van Eldik, R. Berry, and R. Vassar. 2006. Intraneuronal beta-amyloid aggregates, neurodegeneration, and neuron loss in transgenic mice with five familial Alzheimer's disease mutations: potential factors in amyloid plaque formation. J Neurosci. 26:10129-40.
Perry, G., A. D. Cash, and M. A. Smith. 2002. Alzheimer Disease and Oxidative Stress. J Biomed Biotechnol. 2:120-123.
Polidori, M. C., H. R. Griffiths, E. Mariani, and P. Mecocci. 2007. Hallmarks of protein oxidative damage in neurodegenerative diseases: focus on Alzheimer's disease. Amino Acids. 32:553-9.
Powell, M. W., D. E. Carnegie, and T. J. Burke. 2004. Reversal of diabetic peripheral neuropathy and new wound incidence: the role of MIRE. Adv Skin Wound Care. 17:295-300.
Powell, M. W., D. H. Carnegie, and T. J. Burke. 2006. Reversal of diabetic peripheral neuropathy with phototherapy (MIRE) decreases falls and the fear of falling and improves activities of daily living in seniors. Age Ageing. 35:11-6.
Pschenitzka, F., and J. C. Sturm. 2001. Excitation mechanisms in dye-doped organic light-emitting devices. Applied Physics Letters. 79:4354-4356.
Selkoe, D. J. 2001. Alzheimer's disease: genes, proteins, and therapy. Physiol Rev. 81:741-66.
Sherrington, R., S. Froelich, S. Sorbi, D. Campion, H. Chi, E. A. Rogaeva, G. Levesque, E. I. Rogaev, C. Lin, Y. Liang, M. Ikeda, L. Mar, A. Brice, Y. Agid, M. E. Percy, F. Clerget-Darpoux, S. Piacentini, G. Marcon, B. Nacmias, L. Amaducci, T. Frebourg, L. Lannfelt, J. M. Rommens, and P. H. St George-Hyslop. 1996 Alzheimer's disease associated with mutations in presenilin 2 is rare and variably penetrant. Hum Mol Genet. 5:985-8.
Sommer, A. P., A. L. Pinheiro, A. R. Mester, R. P. Franke, and H. T. Whelan. 2001. Biostimulatory windows in low-intensity laser activation: lasers, scanners, and NASA's light-emitting diode array system. J Clin Laser Med Surg. 19:29-33.
Wong-Riley, M. T., H. L. Liang, J. T. Eells, B. Chance, M. M. Henry, E. Buchmann, M. Kane, and H. T. Whelan. 2005. Photobiomodulation directly benefits primary neurons functionally inactivated by toxins: role of cytochrome c oxidase. J Biol Chem. 280:4761-71.
Wu, C. C., J. C. Sturm, R. A. Register, J. Tian, E. P. Dana, and M. E. Thompson. 1997. Efficient organic electroluminescent devices using single-layer doped polymer thin films with bipolar carrier transport abilities. Ieee Transactions on Electron Devices. 44:1269-1281.
Yu, G., and A. J. Heeger. 1995. Charge Separation and Photovoltaic Conversion in Polymer Composites with Internal Donor-Acceptor Heterojunctions. Journal of Applied Physics. 78:4510-4515.
Yu, W., J. O. Naim, and R. J. Lanzafame. 1997. Effects of photostimulation on wound healing in diabetic mice. Lasers Surg Med. 20:56-63.
Zhu, P. J., and V. A. Chiappinelli. 1999. Nicotine modulates evoked GABAergic transmission in the brain. J Neurophysiol. 82:3041-3045. Baynes, J. W. 1991. Diabetes 40, 401-412.
Baynes, J. W. and Thorpe, S. R. 1999. Diabetes. 48, 1-9.
Beauvoit, B., Katai, T., and Chance, B. 1994. Biophys J. 67, 2501-25 10. Capla, J. M. et al. 2007. Plastic Reconstructive Surgery 119, 59-70.
Castello, P. R., David, P. S., McClure, T., Crook, Z., and Poyton, R. O. (2006). Cell Metabolism 3, 277-287.
Chandel, N. S., McClintock, D. S., Feliciano, C. E., Wood, T. M., Melendez, J. A.,
Rodriguez, A. M., and Schumacker, P. T. (2000).
Conlan, M. J., Rapley, J. W., and Cobb, C. M. 1996. J. Clin. Periodont. 23, 492-496.
DCCT. 1993. The Diabetes Control and Complications Trial Research Group. New. Eng. J. Med. 329, 977-986.
DCCT. 1995. Ann. Neurol. 38, 869-880. DCCT/EDIC. 2002. JAMA 287, 2563-2569.
Delellis, S., Carnegie, D. E., and Burke, T. J. 2005. J. Amer. Podiatric Med. Assoc. 95, 143-147.
Dirmeier, R., O'Brien, K. M., Engle, M., Dodd, A., Spears, E., and Poyton, R. O. (2002). J. Biol. Chem. 277, 34773-34784.
Eells, J. Y., Henry, M. M., Summerfelt, P., Wong-Riley, M. T., Buchmann, E. V., Kane, M., Whelan, N. T., and Whelan, H. T. 2004. Proc. Natl. Acad. Sci USA, 100, 3439-3444.
Harkless, L. B., Dellellis, S., Carnegie, D., and Burke, T. J. 2006. J. Diabetes Complications. 20, 81-87.
Karu, T. 1999. J. Photochem. Photobiol. 49, 1-17.
Karu, T. I., Pyatibrat, L. V., and Kalendo, G. s. 2004. Photochem Photobiol Sci 3, 211-216.
Karu, T. I., Pyatibrat, L. V., Kolyakov, S. F. 2005. Photochem Photobiol. 81 (2), 98-106.
Lee, I., Salomon, A. R., Ficarro, S., Mathes, I., Lottspeich, F., Grossman, L. I., and Huttemann, M. (2005). J. Biol. Chem. 280, 6094-6100.
Lowell, B. B. and Schulman, G. I. 2005. Science 307, 384-387.
Palm, F. 2006. Clinical Experimental Pharmacology and Physiology. 33, 997-1001.
Pop-Busui, R., Sima, A., and Stevens, M. 2006. Diabetes Metabolism Research Review. 22, 257-273.
Powell, M., Carnegie, D. and Burke, T. 2004. Advances in Skin and Wound Care. 17, 295-300.
Powell, M. W., Carnegie, D. H., and Burke, T. J. 2006. Age Ageing 35, 454.
Poyton, R. O., C. E. Trueblood, R. M. Wright and L. E. Farrell (1988) Ann. N.Y. Acad. Sci. 550:289-307.
Poyton, R. O. (1998) Assembling a time bomb—cytochrome c oxidase and disease. Nature Genetics 20:316-317.
Poyton, R. O. and J. E. McEwen (1996) Annu Rev. Biochem. 65:563-607.
Prabu, S. K., Anandatheerthavarada, H. K., Raza, H., Srinivasan, S., Spear, J. F., and Avadhani, N. G. (2006). J. Biol. Chem. 281, 2061-2070.
Rolo, A. P. and Palmeira, C. M. 2006. Toxicol and Applied Pharmacology 212, 167-178.
Ryan, M. T., and Hoogenraar, N. J. 2007. Annu Rev. Biochem. 76, 4.1-4.22 Sommer, A. P., Pinheiro, A. L., Mester, A. R., Franke, R. P., and Whelan, H. T. 2001. J. Clin. Laser Med. Surg. 19, 29-33.
Tachtsidis, I., Tisdall, M., Leung, T. S., Cooper, C. E., Delpy, D. T., Smith, M., and Elwell, C. E. 2007. Physiol. Meas. 28, 199-211.
Takahashi, H., Shin, Y., Cho, S. J., Zago, W. M., Nakamura, T., Gu, Z., Ma, Y., Furukawa, H., Liddington, R., Zhang, D., Tong, G., Chen, H. S., and Lipton, S. A. (2007). Neuron 53, 53-64.
Whelan et al. 2001. J. Clin. Laser Med. Surg. 19, 305-314.
Whelan et al. 2002. J. Clin. Laser Med. Surg. 20, 319-324.
Winterle, J. S., and Einarsdottir, 0. 2006. Photochem Photobiol. 82: 711-719.
Wong-Riley, M. T., Bai, X., Buchmann, E., and Whelan, H. T. 2001. Neuroreport 12, 3033-3037.
Wong-Riley, M. T. T., Liang, H. L., Eelles, J. T., Chance, B., Henry, M.m., Buchmann, E., Kane, M., and Whelan, H. T. 2005. J. Biol. Chem. 280, 4761-477 1.
Yu, W., Naim, J. 0., and Lanzafame, R. J. 1997. Lasers Surg. Med. 20, 56-63.

The references cited herein are hereby incorporated by reference in their entirety. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

1. A medical treatment device, comprising:
a patch wearable by a mammal, the patch having a tissue facing surface and including a light source operable to emit outwardly from the tissue facing surface electromagnetic radiation in a visible portion of the electromagnetic spectrum from about 375 nm to 625 nm, the electromagnetic radiation being substantially free of electromagnetic radiation in the portion of the electromagnetic spectrum between 650 nm and 700 nm.

2. The device of claim 1, wherein the light source is operable to emit the electromagnetic radiation in the visible portion of the electromagnetic spectrum with at least one of a surface power density of about 10 mW/cm² to about 10 W/cm² or a total power output of about 25 mW to about 100 W measured adjacent to the tissue facing surface.

3. The device of claim 1, wherein the tissue facing surface of the patch has a surface area of from about one square inch to about ten square inches.

4. The device of claim 3, wherein the patch is conformable to a neck of a human.

5. The device of claim 1, wherein the tissue facing surface of the patch has a diameter of about 0.5 to about 4.0 inches.

6. The device of claim 1, further comprising:
a biocompatible adhesive carried by the patch to selectively removably attach the patch to a bodily tissue of the mammal.

7. The device of claim 1, further comprising:
a battery integral to the patch and electrically coupled to supply electrical power to the light source.

8. The device of claim 7, wherein the battery is sized to provide only a single-use treatment.

9. The device of claim 1, further comprising:
a controller coupled to selectively control the light source.

10. The device of claim 9, wherein the controller is configured to pulsate the electromagnetic radiation.

11. The device of claim 9, further comprising:
at least one sensor positioned to sense at least one parameter of a treatment and communicatively coupled to the controller to provide signals thereto, and wherein the controller is configured to adjust at least one operational parameter based on the signals from the at least one sensor.

12. The device of claim 11, wherein the treatment parameter that the at least one sensor senses includes at least one of a patient characteristic, a selected applied power density, a target time interval, a power density/timing profile, or a temperature.

13. The device of claim 1, further comprising:
one or more optical filters that remove a portion of the electromagnetic radiation having wavelengths between 650 nm and 700 nm.

14. The device of claim 1, wherein the light source is operable to emit outwardly from the tissue facing surface the electromagnetic radiation in the visible portion of the electromagnetic spectrum from about 375 nm to about 650 nm and which is substantially free of wavelengths greater than about 675 nm, and which has a peak energy transmission at or within 10 nm of a wavelength of about 400 nm, 550 nm, about 560 nm, about 570 nm, about 580 nm, about 590 nm, about 600 nm, or about 610 nm; or which has an energy distribution for which 80% or 90% if the energy is found within the wavelengths of 500 nm to 625 nm.

15. A method, comprising:
supplying a device comprising a light source in the form of a multi- or single-use patch; and
operating the device to emit electromagnetic radiation in a visible portion of the electromagnetic spectrum from about 375 nm to 625 nm, the electromagnetic radiation being substantially free of electromagnetic radiation in the portion of the electromagnetic spectrum between 650 nm and 700 nm.

16. The method of claim 15, wherein operating the device includes:
operating the device to emit the electromagnetic radiation in the visible portion of the electromagnetic spectrum with at least one of a surface power density of about 10 mW/cm² to about 10 W/cm² or a total power output of about 25 mW to about 100 W measured adjacent to a surface of the device;
causing a controller to selectively control operation of the plurality of organic light emitting diodes;
operating the device to emit the electromagnetic radiation for a period of time from about 10 seconds to about two hours or more; or
operating the device to emit the electromagnetic radiation continuously or at a pulse frequency of about 4 to about 10,000 Hz.

17. The method of claim 15, wherein supplying the device includes:
supplying the device comprising the light source in the form of a multi- or single-use patch having at least one of a surface area of from about one square inch to about ten square inches or a diameter of about 0.5 to about 4.0 inches, and bearing an adhesive substance on an exterior surface thereof;
supplying the device comprising one or more optical filters to remove a portion of electromagnetic radiation having wavelengths greater than about 650 nm;
supplying the device comprising the light source, wherein the light source comprises a plurality of organic light emitting diodes; or
supplying the device comprising the light source wherein the device comprises a controller coupled to selectively control the light source.

18. A method for increasing mitochondrial nitrite reductase activity, increasing Cytochrome c oxidase activity, increasing nitric oxide production, or increasing tissue blood flow in a tissue of a mammalian subject, comprising:
exposing said tissue to electromagnetic radiation in a visible portion of the electromagnetic spectrum from about 375 nm to 625 nm, the electromagnetic radiation being substantially free of electromagnetic radiation in the portion of the electromagnetic spectrum between 650 nm and 700 nm, by externally applying the electromagnetic radiation to the mammalian subject using a medical treatment device comprising:
a patch wearable by the subject, the patch having a tissue facing surface and including a light operable to emit outwardly from the tissue facing surface electromagnetic radiation in a visible portion of the electromagnetic spectrum from about 375 nm to about 625 nm, the electromagnetic radiation being substantially free of the electromagnetic radiation in the portion of the electromagnetic spectrum between 650 nm and 700 nm.

19. The method of claim 18, wherein the tissue is selected from the group consisting of: a hypoxic or ischemic tissue; a tissue affected by diabetic peripheral neuropathy; a tissue of the central nervous system, including brain tissue or spinal cord tissue; a tissue affected by hypoxia, ischemia, oxidative stress or neurodegeneration; and a tissue located some distance from the tissue affected by hypoxia, ischemia, oxidative stress or neurodegeneration.

20. The method of claim 18, wherein:
the tissue is exposed to about 0.5 to about 40 joules/cm² of the electromagnetic radiation;
the tissue is exposed to about 1 to about 20 joules/cm² of the electromagnetic radiation;
the tissue is exposed to a power density of the electromagnetic radiation of about 0.01 mW/cm² to about 1 W/cm²;
the tissue is exposed to a power density of the electromagnetic radiation of about 0.01 mW/cm² to about 100 mW/cm²;
the tissue is exposed to a power density of the electromagnetic radiation of about 0.5 mW/cm² to about 8 mW/cm²;
the tissue is exposed to electromagnetic radiation modulated or pulsed at a frequency of about 4 Hz to about 10,000 Hz;
the tissue is exposed to the electromagnetic radiation over a treatment period of from about 10 seconds to about two hours or more in length; or
the tissue is exposed to the electromagnetic radiation at a frequency of treatment of once- or twice-a-day, 1-, 2-, 3-, 4-, or 5-times a week, or once- or twice-a-month.

21. The method of claim 21, wherein said subject is also administered a compound that modulates nitric oxide levels in said subject.

22. A method for treating or preventing reduced blood flow, hypoxia, ischemia, oxidative stress, or neurodegeneration, or for increasing cerebral blood flow, in a mammalian subject, comprising:
externally applying electromagnetic radiation in a visible portion of the electromagnetic spectrum from about 375 nm to 625 nm, the electromagnetic radiation being substantially free of electromagnetic radiation in the portion of the electromagnetic spectrum between 650 nm and 700 nm to said subject using a medical treatment device, comprising:
a patch wearable by the subject, the patch having a tissue facing surface and including a light source operable to emit outwardly from the tissue facing surface electromagnetic radiation in a visible portion of the electromagnetic spectrum from about 375 nm to about 625 nm, the electromagnetic radiation being substantially free of electromagnetic radiation in the portion of the electromagnetic spectrum between 650 nm and 700 nm.

23. The method of claim 22, wherein said mammalian subject has a disease or disorder selected from the group consisting of: stroke; cerebral ischemia; migraine; multiple sclerosis; amylotrophic lateral sclerosis; epilepsy; Alzheimer's disease; dementia, including Alzheimer-type dementia, cerebrovascular dementia, senile dementia, fronto-temporal dementia, and dementia resulting from AIDS; traumatic brain injury; physical trauma to the central nervous system, including traumatic brain injury, crush or compression injury to the brain, spinal cord, nerves, or retina; a neurodegenerative disease; Parkison's disease; Huntington's disease; ischemia/reperfusion disease; tissue injury; cardiovascular diseases, including atherosclerosis and hypertension; non-diabetic peripheral neuropathies; diabetes and diabetic complications of the eye (e.g., macular degeneration), kidney, and nerves, including diabetic peripheral neuropathy; non-diabetic peripheral neuropathies; inflammation; arthritis; radiation injury; aging; burns; spine/back disease, including herniated discs; peripheral vascular disease; vasospasm; a deficit in cognition or memory; and obesity.

24. The method of claim 22, wherein the subject has a neurodegenerative disease or disorder and the subject's brain or one or more of the subject's carotid arteries and/or vertebral arteries is exposed to the electromagnetic radiation by positioning the device on the subject's head or neck, or under the ear or behind the jaw bone of the subject.

25. The method of claim 22, wherein the subject has Alzheimer's disease and one or more of the subject's carotid arteries and/or vertebral arteries is exposed to the electromagnetic radiation by positioning the device on the subject's neck or under the ear or behind the jaw bone of the subject.

26. The method of claim 22, wherein:
the electromagnetic radiation has a bandwidth of about 50 nm;
the light source provides a unit dose of electromagnetic radiation in an amount of from about 0.5 to about 40 joules/cm² per treatment;
the light source provides a unit dose of electromagnetic radiation in an amount from about 5 to about 50 joules/cm²/day;
the electromagnetic radiation is monochromatic light;
the electromagentic radiation principally comprises wavelengths from 550 nm to 600 nm;
the electromagnetic radiation principally comprises wavelengths from 575 nm to 600 nm;
the subject is contacted with the electromagnetic radiation over a treatment period of from about 10 seconds to about two hours or more in length;
the subject is contacted with the electromagnetic radiation from once- or twice-a-day; 1-, 2-, 3-, 4-, or 5-times a week, or once- or twice-a month;
the electromagentic radiation has a peak energy emission at a wavelength of about 400 nm, 500 nm, 510 nm, 520 nm, 530 nm, 540 nm, 550 nm, 560 nm, 570 nm, 580 nm, 590 nm, 600 nm, 610 nm, or within 10 nm of any one of these values;
the electromagnetic radiation has an energy distribution for which 80% of the energy is found within the wavelengths of 550 nm to 600 nm; or
the electromagnetic radiation has an energy distribution for which 90% of the energy is found within with the wavelengths of 550 nm to 600 nm.

27. The method of claim 22, wherein said subject is also administered a compound that modulates nitric oxide levels in said subject.
